(19) United States
(12) Patent Application Publication
(10) Pub. No.: US 2025/0281462 A1
(43) Pub. Date: Sep. 11, 2025

(54) THERAPEUTICALLY EFFECTIVE ORAL ADMINISTRATION OF A 2 ARYLBENZIMIDAZOLE

(71) Applicant: Tranquis Therapeutics, Inc., Palo Alto, CA (US)

(72) Inventors: Jonas O'Gara HANNESTAD, San Mateo, CA (US); Steven August SMITH, San Jose, CA (US); Sanjay Kumar KAKKAR, London (GB)

(21) Appl. No.: 19/019,406

(22) Filed: Jan. 13, 2025

Related U.S. Application Data
(63) Continuation of application No. 18/571,177, filed on Dec. 15, 2023, now abandoned.
(60) Provisional application No. 63/211,636, filed on Jun. 17, 2021.
(60) Provisional application No. 63/300,551, filed on Jan. 18, 2022.

(51) Int. Cl. A61K 31/4184; A61K 9/00; A61P 25/16; A61P 25/28

(52) U.S. Cl. CPC A61K 31/4184; A61K 9/0053; A61P 25/16; A61P 25/28

(57) ABSTRACT

Methods are provided for reducing neuroinflammation and/or treating a neurodegenerative disease in a subject. The methods comprise orally administering to a subject with neuroinflammation and/or a neurogenerative disease a pharmaceutical composition comprising the compound of formula (I)

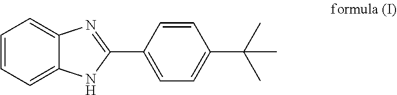 [SMILES: CC(C)(C)c1ccc(-c2nc3ccccc3[nH]2)cc1]

(TQS-168), or a pharmaceutically acceptable salt thereof, in amount that provides defined plasma and brain exposures of TQS-168 and/or an active metabolite following administration.

### CROSS REFERENCE TO RELATED APPLICATIONS

This application is a continuation of U.S. application Ser. No. 18/571,177, filed on Dec. 15, 2023, which is the National Stage of International Application No. PCT/US2022/034012, filed Jun. 17, 2022, which claim the benefit of and priority to U.S. Provisional application Nos. 63/211,636, filed on Jun. 17, 2021, and 63/300,551, filed on Jan. 18, 2022, each of which are incorporated herein by reference in their entireties for all purposes.

### 1. BACKGROUND OF THE INVENTION

The 2-arylbenzimidazole compound TQS-168—2-(4-tert-butylphenyl)-1H-benzimidazole—previously known as ZLN-005, is an activator of Ppargc1α (PGC-1α) gene expression. Zhang et al., Diabetes 62:1297-1307 (2013). When administered orally at 25-50 mg/kg to mice, TQS-168 has been shown to suppress myeloid-mediated inflammation and reduce disease severity in murine models of neurodegenerative diseases, including Parkinson's disease, Alzheimer's disease, and amyotrophic lateral sclerosis (ALS). See U.S. Pat. No. 10,272,070. When administered orally to mice at 25 mg/kg, TQS-168 has also been shown to suppress metabolic dysfunction in microglia in older mice, inhibit inflammatory cytokine production in microglia in older mice, suppress systemic inflammation in older mice, and alleviate behavioral dysfunction in older mice. See U.S. Pat. No. 10,653,669. TQS-168 and structurally related 2-arylbenzimidazoles have also been shown to be effective in treating systemic immune activation. See WO 2021/262617.

TQS-168 is highly insoluble. In the animal model experiments reported in U.S. Pat. No. 10,272,070 and 10,653,669, TQS-168 was prepared as an oral suspension and administered to experimental animals by oral gavage. Plasma and brain concentrations of the compound after administration were not reported, providing no pharmacokinetic (PK) information.

In order to establish an effective oral dosing regimen suitable for human patients, there is a need to define the plasma and brain concentrations and total exposures of TQS-168 that provide pharmacodynamic benefit.

### 2. SUMMARY OF THE INVENTION

We have now demonstrated that TQS-168 induces PGC-1α protein expression in vitro in a murine myeloid cell line, BV2, at concentrations ranging from 0.7 μM (175.21 ng/mL) to 20 μM (5006 ng/mL), and that TQS-168 suppresses LPS-induced secretion of pro-inflammatory cytokines from BV2 cells and human primary myeloid cells in vitro at concentrations ranging from 0.3 μM (75.09 ng/mL) to 20 μM (5006 ng/mL).

These in vitro experiments predict that plasma free-drug and brain concentrations of TQS-168 in the range of 0.3-20 μM (75.09-5006 ng/mL) should suppress myeloid-mediated neuroinflammation.

When administered orally at 25-50 mg/kg, TQS-168 was previously shown to suppress myeloid-mediated inflammation and reduce disease severity in murine models of neurodegenerative diseases, including Parkinson's disease, Alzheimer's disease, and amyotrophic lateral sclerosis (ALS). U.S. Pat. No. 10,272,070. We have now measured plasma, liver, and brain concentrations of TQS-168 after single oral dose administration of 25 mg/kg to mice, the dose previously found to provide therapeutic effect. At this previously established effective dose, mean plasma Cₘₐₓ of TQS-168 was 93.4 ng/ml, or 0.37 μM, and mean brain Cₘₐₓ was higher, at 542.0 ng/ml, or 2.16 μM. These concentrations are within the range of concentrations shown to induce PGC-1α protein expression and to reduce LPS-mediated inflammatory cytokine release in vitro. These in vivo data confirm that plasma concentrations of TQS-168 in the range of 0.3 μM-20 μM should suppress myeloid-mediated neuroinflammation. The evidence of accumulation in brain suggests that plasma concentrations of TQS-168 lower than 0.37 μM may also be effective in treating neuroinflammation.

We have also demonstrated that a primary phase 1 metabolite of TQS-168, TQS-621, potently inhibits LPS-induced IL-6 and TNFα secretion from primary human PBMCs. These data demonstrate that at least some of the therapeutic effects observed after oral administration of TQS-168 can likely be attributed to the activity of metabolite TQS-621.

We have also conducted a phase 1 human clinical trial and measured plasma concentrations of TQS-168 and active metabolite TQS-621 with three different formulations and demonstrated that pharmacodynamically relevant plasma concentrations can be achieved with oral suspensions of several solid formulations of the API.

Accordingly, in a first aspect, methods are provided for reducing neuroinflammation and/or treating a neurodegenerative disease in a subject. The method comprises:
orally administering to a subject with neuroinflammation and/or a neurogenerative disease at least one dose of a pharmaceutical composition comprising the compound of formula (I)

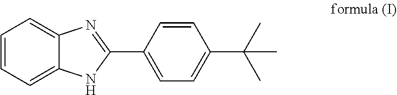 [SMILES: CC(C)(C)c1ccc(-c2nc3ccccc3[nH]2)cc1]

(TQS-168), or a pharmaceutically acceptable salt thereof, in amount that provides, after administration,
a mean peak concentration (Cₘₐₓ) of TQS-168 in plasma of at least 750 ng/mL.

In various embodiments, TQS-168 or salt thereof is administered in an amount that provides, following administration, a mean plasma Cₘₐₓ of TQS-168 of at least 1000 ng/mL, at least 1250 ng/mL, at least 1500 ng/mL, or at least 1750 ng/mL.

In some embodiments TQS-168 or salt thereof is administered in an amount that provides, following administration, an AUC₀₋ₜ of at least 3000 ng·hr/ml, at least 4000 ng·hr/ml, at least 5000 ng·hr/ml, at least 5500 ng·hr/ml, at least 6000 ng*hr/ml, or at least 7,000 ng*hr/ml. In particular embodiments, TQS-168 or salt thereof is administered in an amount that provides, following administration, an AUC₀₋ₜ of about 6000 ng·hr/ml.

In various embodiments, the time to plasma Cmax (Tmax) of TQS-168 is no more than 2 hours, no more than 90 minutes, or no more than 75 minutes. In particular embodiments, the TQS-168 plasma Tmax is about 60 minutes.

In a second aspect, methods are provided for reducing neuroinflammation and/or treating a neurodegenerative disease in a human subject. The method comprises: orally administering to a subject with neuroinflammation and/or a neurogenerative disease a pharmaceutical composition comprising the compound of formula (I)

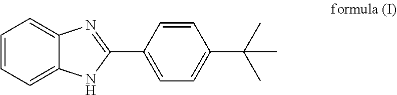 [SMILES: CC(C)(C)c1ccc(-c2nc3ccccc3[nH]2)cc1]

(TQS-168), or a pharmaceutically acceptable salt thereof, in amount that provides following administration,
a mean peak plasma concentration (Cₘₐₓ) of the compound of Formula (II)

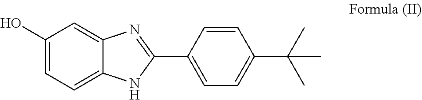 [SMILES: CC(C)(C)c1ccc(-c2nc3cc(O)ccc3[nH]2)cc1]

(TQS-621) of at least 1000 ng/mL.

In some embodiments, TQS-168 or salt thereof is administered in an amount that provides, following administration, a plasma Cₘₐₓ of TQS-621 of 200-2750 ng/mL, 300-2200 ng/mL, or 400-1800 ng/mL.

In another aspect, methods are provided for reducing neuroinflammation and/or treating a neurodegenerative disease in a subject. The method comprises:
orally administering to a subject with neuroinflammation and/or a neurogenerative disease a pharmaceutical composition comprising the compound of formula (I)

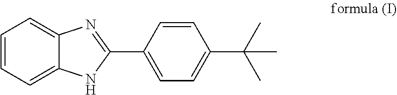 [SMILES: CC(C)(C)c1ccc(-c2nc3ccccc3[nH]2)cc1]

(TQS-168), or a pharmaceutically acceptable salt thereof, in amount that provides following administration,
(a) a mean peak concentration (Cₘₐₓ) of TQS-168 in plasma of at least 750 ng/mL, with
(b) a mean time to Cₘₐₓ (Tₘₐₓ) of TQS-168 in plasma of no more than 75 minutes; and
(c) a mean peak concentration (Cₘₐₓ) of the compound of Formula (II)

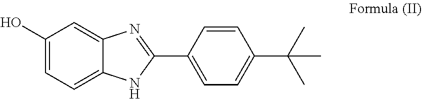 [SMILES: CC(C)(C)c1ccc(-c2nc3cc(O)ccc3[nH]2)cc1]

(TQS-621) in plasma of at least 1000 ng/mL, with
(d) a mean time to Cₘₐₓ (Tₘₐₓ) of TQS-621 in plasma of no more than 4 hours.

In some embodiments of the methods herein, TQS-168, or salt thereof, is administered in a daily oral dose of 200-800 mg, 300-700 mg, 400-600 mg, or 400-500 mg. In particular embodiments, TQS-168, or salt thereof, is administered in a daily oral dose of 400 mg or 450 mg.

In various embodiments of the methods described herein, TQS-168 or salt thereof is administered in a liquid suspension. In certain embodiments, TQS-168 or salt thereof is administered in a liquid solution.

In certain embodiments, TQS-168 or salt thereof is administered in a solid dosage form. In particular solid form embodiments, TQS-168 or salt thereof is crystalline. In particular solid form embodiments, embodiments, TQS-168 or salt thereof is amorphous, and in specific amorphous embodiments, is a spray-dried dispersion or hot melt extrusion. In certain embodiments, the solid dosage form is a sachet, a capsule, or a tablet.

In various embodiments, the subject has a neurodegenerative disease selected from a motor neuron disease, amyotrophic lateral sclerosis (ALS), Alzheimer's disease, vascular dementia, frontotemporal degeneration (frontotemporal dementia), dementia with Lewy bodies, Parkinson's disease, Huntington's disease, demyelinating disease, and multiple sclerosis (MS). In particular embodiments, the subject has a motor neuron disease. In specific embodiments, the subject has ALS. In particular embodiments, the subject has Alzheimer's disease.

In some embodiments, the subject is at least 40 years old and does not have a prior-diagnosed neurodegenerative disease. In particular embodiments, the subject is at least 60 years old or at least 65 years old.

### 3. BRIEF DESCRIPTION OF THE SEVERAL VIEWS OF THE DRAWINGS

These and other features, aspects, and advantages of the present invention will become better understood with regard to the following description, and accompanying drawings, where:

FIG. 1 is a Western blot showing induction of PGC-1α protein expression in the BV2 murine microglial cell line after incubation with TQS-168 at 20 μM in vitro.

FIG. 2 is a Western blot showing the dose response of the increase in PGC-1α protein expression in BV2 cells contacted with TQS-168 in vitro.

FIG. 3 is a bar graph quantifying protein expression levels measured from scans of the Western Blot shown in FIG. 2. “PD” indicates PD169316, a p38 MAPK inhibitor. “Rosi” refers to rosiglitazone (AVANDIA), a PPARγ agonist.

FIGS. 4a-4f show the in vitro secretion of cytokines by BV2 cells after LPS activation in the presence of two positive controls, a negative control, and TQS-168 at 4 different concentrations. The graphs show dose-responsive inhibition of LPS-stimulated TNFα and IL-6 cytokine release by TQS-168 at various concentrations. Cytokine secretion was measured using a cytokine bead array (CBA) fluorescence-activated cell sorting (FACS) assay.

FIGS. 5a-5b show inhibition of TNFα production by LPS-stimulated BV2 myeloid cells in vitro by 5 μM (FIG. 5a) and 20 μM (FIG. 5b) TQS-168 treatments, respectively.

FIGS. 6a-6d show the dose response of TQS-168-mediated inhibition of pro-inflammatory cytokine TNFα release from LPS-stimulated microglia BV2 cells at 24 hours, using an ELISA. FIG. 6a and FIG. 6c show the absolute (FIG. 6a) and relative (FIG. 6c) inhibition of TNFα secretion by BV2 cells stimulated with 0.3 ng/mL of LPS. FIG. 6b and FIG. 6d show the absolute (FIG. 6b) and relative (FIG. 6d) inhibition of TNFα secretion by BV2 cells stimulated with 1 ng/mL of LPS.

FIG. 7 shows inhibition of LPS-induced TNFα secretion in human PBMC cells treated in vitro with TQS-168 at various concentrations.

FIGS. 8a-8c show the average plasma (FIG. 8a), liver (FIG. 8b), and brain (FIG. 8c) concentrations of TQS-168 at various time points following a single oral dose of 25 mg/kg TQS-168 in mice.

FIGS. 9a-9c show the plasma concentrations of TQS-168 at various time points following a single oral dose of 50 mg/kg in wild type mice.

FIG. 10 shows the plasma concentration of TQS-168 at various time points following a single intravenous (IV) dose of 0.5 mg/kg in three individual mice.

FIG. 11 shows the mean plasma concentration of TQS-168 at various time points following a single intravenous (IV) dose of 0.5 mg/kg in three mice.

FIG. 12a shows the plasma concentrations of TQS-168 at various time points following a single oral dose of 50, 150, or 500 mg/kg in rats. FIG. 12b shows the mean brain concentration of TQS-168 at various time points after a single oral dose of 500 mg/kg in rats.

FIG. 13 shows dose-dependent Cₘₐₓ (ng/mL) values of TQS-168 after oral dosing of 50 mg/kg, 150 mg/kg, and 500 mg/kg in rats.

FIG. 14 shows the dose-dependent AUC (ng*min/mL) of TQS-168 after oral dosing of 50 mg/kg, 150 mg/kg, and 500 mg/kg in rats.

FIG. 15 shows the plasma concentrations of TQS-168 at various times points following a single intravenous (IV) dose of 0.5 mg/kg in three rats.

FIG. 16 shows the average plasma concentrations of TQS-168 at various times points following single intravenous (IV) dose of 0.5 mg/kg in rats.

FIG. 17 shows the plasma concentrations of TQS-168 at various times points following single intravenous (IV) dose of 0.5 mg/kg in three dogs.

FIG. 18 shows the average plasma concentrations of TQS-168 at various times points following single intravenous (IV) dose of 0.5 mg/kg in dogs.

FIG. 19A shows average plasma concentrations of TQS-168 after an oral dose of TQS-168 of 45 mg/kg in mice. FIG. 19B shows average brain concentrations of TQS-168 after an oral dose of TQS-168 of 45 mg/kg in mice.

FIG. 20 illustrates the phase 1 metabolites from liver metabolism of TQS-168 following oral administration.

FIG. 21A shows absolute inhibition of LPS-stimulated IL-6 secretion by TQS-168 metabolite TQS-621 from previously frozen PBMCs obtained from a first healthy human volunteer donor. FIG. 21B shows the relative inhibition of IL-6 inhibition expressed as percentage activity.

FIG. 22A shows absolute inhibition of LPS-stimulated IL-6 secretion by TQS-168 metabolite TQS-621 from previously frozen PBMCs obtained from a second healthy human donor. FIG. 22B shows the relative inhibition of IL-6 inhibition expressed as percentage activity.

FIG. 23A shows absolute inhibition of LPS-stimulated TNFα secretion by TQS-168 and metabolite TQS-621 from previously frozen PBMCs of the first donor. FIG. 23B shows the relative inhibition of IL-6 inhibition expressed as percentage activity.

FIG. 24A shows the absolute inhibition of LPS-stimulated TNFα secretion by TQS-168 and metabolite TQS-621 from previously frozen PBMCs obtained from the second donor. FIG. 24B shows relative inhibition of TNFα inhibition expressed as percentage activity.

FIGS. 25A-25C plot plasma concentration of TQS-168 over time after a single oral dose of TQS-168 in mice administered 50 mg/mL TQS-168 in different formulations.

FIGS. 26A-26C plot plasma concentration of TQS-621 over time after a single oral dose in mice administered 50 mg/mL TQS-168 in different amounts.

FIGS. 27A-B plot plasma concentration of TQS-168 over time after a single oral dose of 60 mg, 180 mg or 540 mg TQS-168 methylcellulose powder suspension formulation in humans. FIG. 27A is a linear plot. FIG. 27B is a logarithmic plot.

FIGS. 28A-B plot plasma concentration of metabolite TQS-621 over time after a single oral dose of 60 mg, 180 mg or 540 mg TQS-168 methylcellulose powder suspension formulation in humans. FIG. 28A is a linear plot. FIG. 28B is a logarithmic plot.

FIGS. 29A-B plot plasma concentration of TQS-168 and metabolite TQS-621 over time after a single oral dose of 60 mg TQS-168 methylcellulose powder suspension oral formulation in humans. FIG. 29A is a linear plot. FIG. 29B is a logarithmic plot.

FIGS. 30A-B plot plasma concentration of TQS-168 and metabolite TQS-621 over time after a single oral dose of 180 mg TQS-168 methylcellulose powder suspension oral formulation in humans. FIG. 30A is a linear plot. FIG. 30B is a logarithmic plot.

FIGS. 31A-B plot plasma concentration of TQS-168 and metabolite TQS-621 over time after a single oral dose of 540 mg TQS-168 methylcellulose powder suspension oral formulation in humans. FIG. 31A is a linear plot. FIG. 31B is a logarithmic plot.

FIGS. 32A-B plot plasma concentration of TQS-168 over time after a single dose of 60 mg, 180 mg, or 540 mg TQS-168 methylcellulose (MC) powder for oral suspension in the fasted state, 90 mg spray dried dispersion (SDD) powder for oral suspension in the fed state, 180 mg SDD powder for oral suspension in the fasted state, or 180 mg of hot melt extrusion (HME) powder in the fasted state. FIG. 32A is a linear plot. FIG. 32B is a logarithmic plot.

FIGS. 33A-B plot plasma concentration of metabolite TQS-621 over time after a single dose of 60 mg, 180 mg, or 540 mg TQS-168 methylcellulose (MC) powder for oral suspension in the fasted state, 90 mg spray dried dispersion (SDD) powder for oral suspension in the fed state, 180 mg SDD powder for oral suspension in the fasted state, or 180 mg of hot melt extrusion (HME) powder in the fasted state. FIG. 33A is a linear plot. FIG. 33B is a logarithmic plot.

FIGS. 34A-B plot plasma concentration of TQS-168 and metabolite TQS-621 over time after a single dose of 90 mg TQS-168 spray dried dispersion (SDD) powder suspension (oral formulation) in fed state humans. FIG. 34A is a linear plot. FIG. 34B is a logarithmic plot.

FIGS. 35A-B plot plasma concentration of TQS-168 over time after a single dose of 90 mg spray dried dispersion (SDD) powder suspension (oral formulation) in fed and fasted state humans. FIG. 35A is a linear plot. FIG. 35B is a logarithmic plot.

FIGS. 36A-B plot plasma concentration of metabolite TQS-621 over time after a single dose of 90 mg spray dried dispersion (SDD) powder suspension (oral formulation) in fed and fasted state humans. FIG. 35A is a linear plot. FIG. 35B is a logarithmic plot.

FIGS. 37A-B plot plasma concentration of TQS-168 and metabolite TQS-621 over time after a single oral dose of 90 mg TQS-168 spray dried dispersion (SDD) powder suspension (oral formulation) in fasted state humans. FIG. 37A is a linear plot. FIG. 37B is a logarithmic plot.

FIGS. 38A-B plot plasma concentration of TQS-168 over time after a single dose of 90 mg, 180 mg or 270 mg TQS-168 spray dried dispersion (SDD) powder suspension (oral formulation) in fasted state humans. FIG. 38A is a linear plot. FIG. 38B is a logarithmic plot.

FIGS. 39A-B plot plasma concentration of metabolite TQS-621 over time after a single dose of 90 mg, 180 mg or 270 mg TQS-168 spray dried dispersion (SDD) powder suspension (oral formulation) in fasted state humans. FIG. 39A illustrates a linear plot. FIG. 39B illustrates a logarithmic plot.

FIGS. 40A-B plot plasma concentration of TQS-168 over time after a single dose of 90 mg or 120 mg spray dried dispersion (SDD) powder suspension (oral formulation) in fed and fasted state humans respectively (Day 1). FIG. 40A is a linear plot. FIG. 40B is a logarithmic plot.

FIGS. 41A-B plot plasma concentration of TQS-168 over time after seven consecutive days of single daily dose 90 mg or 120 mg spray dried dispersion (SDD) powder suspension (oral formulation) in fed and fasted state humans respectively. FIG. 41A is a linear plot. FIG. 41B is a logarithmic plot.

FIGS. 42A-B plot plasma concentration of metabolite TQS-621 over time after a single dose of 90 mg or 120 mg TQS-168 spray dried dispersion (SDD) powder suspension (oral formulation) in fed and fasted state humans respectively. FIG. 42A is a linear plot. FIG. 42B is a logarithmic plot.

FIGS. 43A-B plot plasma concentration of metabolite TQS-621 over time after seven consecutive days of single dose 90 mg or 120 mg TQS-621 spray dried dispersion powder suspension (oral formulation) in fed and fasted state humans, respectively. FIG. 43A is a linear plot. FIG. 43B is a logarithmic plot.

FIGS. 44A-B plot plasma concentration of TQS-168 over time after a single dose of TQS-168 methylcellulose (MC) powder suspension formulation, spray dried dispersion (SDD) powder suspension formulation, or hot melt extrusion (HME) powder suspension formulation in varying doses in humans. FIG. 44A is a linear plot. FIG. 44B is a logarithmic plot.

FIGS. 45A-B plot plasma concentration of TQS-621 over time after a single dose of TQS-168 methylcellulose (MC) powder suspension formulation, spray dried dispersion (SDD) powder suspension formulation, or hot melt extrusion (HME) powder suspension formulation in varying doses. FIG. 45A is a linear plot. FIG. 45B is a logarithmic plot.

FIGS. 46A-B plot plasma concentration of TQS-168 and metabolite TQS-621 over time after a single dose of 180 mg TQS-168 spray dried dispersion (SDD) powder suspension (oral formulation) in fasted state humans. FIG. 46A is a linear plot. FIG. 46B is a logarithmic plot.

FIGS. 47A-B plot plasma concentration of TQS-168 and metabolite TQS-621 over time after a single dose of 180 mg TQS-168 hot melt extrusion (HME) powder suspension (oral formulation) in fasted state humans. FIG. 34A is a linear plot. FIG. 34B is a logarithmic plot.

FIGS. 48A-B plot plasma concentration of TQS-621 over time after a single dose of TQS-168 methylcellulose (MC) powder suspension formulation, spray dried dispersion (SDD) powder suspension formulation, or hot melt extrusion (HME) powder suspension formulation in varying doses in fasted state humans. FIG. 48A is a linear plot. FIG. 48B is a logarithmic plot.

FIGS. 49A-B plot plasma concentration of TQS-168 over time following consecutive single daily doses 120 mg spray dried dispersion (SDD) powder suspension (oral formulation) in fasted state humans. FIG. 49A is a linear plot. FIG. 49B is a logarithmic plot.

FIGS. 50A-B plot plasma concentration of metabolite TQS-621 over time following consecutive single daily dose of TQS-168 120 mg spray dried dispersion (SDD) powder suspension (oral formulation) in fasted state humans. FIG. 50A is a linear plot. FIG. 50B is a logarithmic plot.

FIGS. 51A-B plot plasma concentration of TQS-168 over time after a single dose of 90 mg, 120 mg or 300 mg spray dried dispersion (SDD) powder suspension (oral formulation) in fed/fasted state humans (Day 1). FIG. 51A is a linear plot. FIG. 51B is a logarithmic plot.

FIGS. 52A-B plot plasma concentration of TQS-168 over time after seven consecutive days of single daily dose 90 mg, 120 mg, or 300 mg spray dried dispersion (SDD) powder suspension (oral formulation) in fed/fasted state humans respectively. FIG. 52A is a linear plot. FIG. 52B is a logarithmic plot.

FIGS. 53A-B plot plasma concentration of metabolite TQS-621 over time after a single dose of 90 mg, 120 mg or 300 mg spray dried dispersion (SDD) powder suspension (oral formulation) in fed/fasted state humans (Day 1). FIG. 53A is a linear plot. FIG. 53B is a logarithmic plot.

FIGS. 54A-B plot plasma concentration of metabolite TQS-621 over time after seven consecutive days of single dose TQS-168 90 mg, 120 mg or 300 mg spray dried dispersion (SDD) powder suspension (oral formulation) in fed/fasted state humans (Day 1). FIG. 54A is a linear plot. FIG. 54B is a logarithmic plot.

FIGS. 55A-B plot plasma concentration of TQS-168 over time after a single dose of TQS-168 methylcellulose (MC) powder suspension formulation, spray dried dispersion (SDD) powder suspension formulation, or hot melt extrusion (HME) powder suspension formulation in varying doses. FIG. 55A is a linear plot. FIG. 55B is a logarithmic plot.

### 4. DETAILED DESCRIPTION OF THE INVENTION

### 4.1. Definitions

Unless defined otherwise, all technical and scientific terms used herein have the meaning commonly understood by a person skilled in the art to which this invention belongs.

The terms “individual,” “host,” and “subject” are used interchangeably, and refer to an animal to be treated, including but not limited to, humans and non-human primates; rodents, including rats and mice; bovines; equines; ovines; felines; and canines. “Mammal” means a member or members of any mammalian species. Non-human animal models, i.e., mammals, non-human primates, murines, lagomorpha, etc. may be used for experimental investigations.

“Patient” refers to a human subject, including a healthy human donor.

The terms “treating,” “treatment,” and grammatical variations thereof are used in the broadest sense understood in the clinical arts. Accordingly, the terms do not require cure or complete remission of disease, and encompass obtaining any clinically desired pharmacologic and/or physiologic effect. Unless otherwise specified, “treating” and “treatment” do not encompass prophylaxis.

The phrase “therapeutically effective amount” refers to the amount of a compound that, when administered to a mammal or other subject for treating a disease, condition, or disorder, is sufficient to effect treatment of the disease, condition, or disorder. The “therapeutically effective amount” may vary depending on the compound, the disease and its severity and the age, weight, etc., of the subject to be treated.

The term “pharmaceutically acceptable salt” refers to a salt that is acceptable for administration to a subject. Examples of pharmaceutically acceptable salts include, but are not limited to: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, palmoate, pectinate, persulfate, phenylpropionate, picrate, pivalate, propionate, succinate, tartrate, thiocyanate, tosylate, and undecanoate.

Other examples of pharmaceutically salts include anions of the compounds of the present disclosure compounded with a suitable cation such as N⁺, NH₄⁺, and NW₄⁺ (where W can be a C₁-C₈ alkyl group), and the like. For therapeutic use, salts of the compounds of the present disclosure can be pharmaceutically acceptable. However, salts of acids and bases that are non-pharmaceutically acceptable may also find use, for example, in the preparation or purification of a pharmaceutically acceptable compound.

Compounds included in the present compositions and methods that are basic in nature are capable of forming a wide variety of salts with various inorganic and organic acids. The acids that can be used to prepare pharmaceutically acceptable acid addition salts of such basic compounds are those that form non-toxic acid addition salts, i.e., salts containing pharmacologically acceptable anions, including but not limited to, malate, oxalate, chloride, bromide, iodide, nitrate, sulfate, bisulfate, phosphate, acid phosphate, isonicotinate, acetate, lactate, salicylate, citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate and pamoate (i.e., 1,1′-methylene-bis-(2-hydroxy-3-naphthoate)) salts.

Compounds included in the present compositions and methods that are acidic in nature are capable of forming base salts with various pharmacologically acceptable cations. Examples of such salts include alkali metal or alkaline earth metal salts and, particularly, calcium, magnesium, sodium, lithium, zinc, potassium, and iron salts.

Compounds included in the present compositions and methods that include a basic or acidic moiety can also form pharmaceutically acceptable salts with various amino acids. The compounds of the disclosure can contain both acidic and basic groups; for example, one amino and one carboxylic acid group. In such a case, the compound can exist as an acid addition salt, a zwitterion, or a base salt.

Ranges: throughout this disclosure, various aspects of the invention are presented in a range format. Ranges include the recited endpoints. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6, should be considered to have specifically disclosed subranges such as from 1 to 3, from 1 to 4, from 1 to 5, from 2 to 4, from 2 to 6, from 3 to 6, etc. as well as individual number within that range, for example, 1, 2, 3, 4, 5, 5.3, and 6. This applies regardless of the breadth of the range.

In this disclosure, “comprises,” “comprising,” “containing,” “having,” “includes,” “including”, and linguistic variants thereof have the meaning ascribed to them in U.S. Patent law, permitting the presence of additional components beyond those explicitly recited.

Unless specifically stated or apparent from context, as used herein the term “or” is understood to be inclusive.

Unless specifically stated or apparent from context, as used herein, the terms “a” “an”, and “the” are understood to be singular or plural. That is, the articles “a” and “an” are used herein to refer to one or to more than one (i.e., to at least one) of the grammatical object of the article. By way of example, “an element” means one element or more than one element.

Unless specifically stated or otherwise apparent from context, as used herein the term “about” is understood as within range of normal tolerance in the art, for example within 2 standard deviations of the mean, and is meant to encompass variations of ±20% or ±10%, more preferably ±5%, even more preferably ±1%, and still more preferably ±0.1% from the stated value. Where a percentage is provided with respect to an amount of a component or material in a composition, the percentage should be understood to be a percentage based on weight, unless otherwise stated or understood from the context.

It should be understood that the order of steps or order for performing certain actions is immaterial so long as the present disclosure remain operable. Moreover, two or more steps or actions can be conducted simultaneously.

The terms “pharmaceutically acceptable excipient,” “pharmaceutically acceptable diluent,” “pharmaceutically acceptable carrier,” and “pharmaceutically acceptable adjuvant” are used interchangeably and refer to an excipient, diluent, carrier, or adjuvant that is useful in preparing a pharmaceutical composition that are generally safe, non-toxic and neither biologically nor otherwise undesirable, and include an excipient, diluent, carrier, and adjuvant that are acceptable for veterinary use as well as human pharmaceutical use. The phrase “pharmaceutically acceptable excipient” includes both one and more than one such excipient, diluent, carrier, and/or adjuvant.

As used herein, the term “sustained release”, “delayed release”, and “controlled release” refer to prolonged or extended release of the therapeutic agent or API of the pharmaceutical formulation. These terms may further refer to composition which provides prolonged or extended duration of action, such as pharmacokinetics (PK) parameters of a pharmaceutical composition comprising a therapeutically effective amount of the active pharmaceutical ingredient as described herein.

Generally, reference to or depiction of a certain element such as hydrogen or H is meant to include all isotopes of that element. For example, if an R group is defined to include hydrogen or H, it also includes deuterium and tritium. Compounds comprising radioisotopes such as tritium, ¹⁴C, ³²P and ³⁵S are thus within the scope of the present technology. Procedures for inserting such labels into the compounds of the present technology will be readily apparent to those skilled in the art based on the disclosure herein.

Unless the specific stereochemistry is expressly indicated, all chiral, diastereomeric, and racemic forms of a compound are intended. Thus, compounds described herein include enriched or resolved optical isomers at any or all asymmetric atoms as are apparent from the depictions. Racemic mixtures of R-enantiomer and S-enantiomer, and enantio-enriched stereomeric mixtures comprising of R- and S-enantiomers, as well as the individual optical isomers can be isolated or synthesized so as to be substantially free of their enantiomeric or diastereomeric partners, and these stereoisomers are all within the scope of the present technology.

The compounds described herein may exist as solvates, especially hydrates, and unless otherwise specified, all such solvates and hydrates are intended. Hydrates may form during manufacture of the compounds or compositions comprising the compounds, or hydrates may form over time due to the hygroscopic nature of the compounds. Compounds of the present technology may exist as organic solvates as well, including DMF, ether, and alcohol solvates, among others. The identification and preparation of any particular solvate is within the skill of the ordinary artisan of synthetic organic or medicinal chemistry.

As described herein, the text refers to various embodiments of the present compounds, compositions, and methods. The various embodiments described are meant to provide a variety of illustrative examples and should not be construed as descriptions of alternative species. Rather, it should be noted that the descriptions of various embodiments provided herein may be of overlapping scope. The embodiments discussed herein are merely illustrative and are not meant to limit the scope of the present technology.

### 4.2. Summary of Experimental Observations

We have demonstrated that TQS-168 induces PGC-1α gene and protein expression in vitro in a murine myeloid cell line, BV2, at concentrations ranging from 0.7 μM to 20 μM, and that TQS-168 suppresses LPS-induced secretion of pro-inflammatory cytokines from BV2 cells and from human primary myeloid cells in vitro at concentrations ranging from 0.3 μM to 20 μM.

These in vitro experiments predict that plasma and brain concentrations of TQS-168 in the range of 0.3-20 μM should suppress myeloid-mediated neuroinflammation.

When administered orally at 25-50 mg/kg, TQS-168 was previously shown to suppress myeloid-mediated inflammation and reduce disease severity in murine models of neurodegenerative diseases, including Parkinson's disease, Alzheimer's disease, and amyotrophic lateral sclerosis (ALS). U.S. Pat. No. 10,272,070. We have measured plasma, liver, and brain concentrations of TQS-168 after single oral dose administration of 25 mg/kg to mice, the dose previously found to provide therapeutic effect. At this previously established effective dose, average plasma Cₘₐₓ of TQS-168 was 93.4 ng/ml, or 0.37 μM, and brain Cₘₐₓ was higher, at 542.0 ng/ml, or 2.16 μM, within the range of concentrations shown to induce PGC-1α protein expression and to reduce LPS-mediated inflammatory cytokine release in vitro. These in vivo data confirm that plasma concentrations of TQS-168 in the range of 0.3 μM-20 μM should suppress myeloid-mediated neuroinflammation. The evidence of preferential accumulation in brain suggests that plasma concentrations of TQS-168 lower than 0.37 μM may also be effective in treating neuroinflammation.

We have also demonstrated that a primary phase 1 metabolite of TQS-168, TQS-621, potently inhibits LPS-induced IL-6 and TNFα secretion from primary human PBMCs. These data demonstrate that at least some of the therapeutic effects observed after oral administration of TQS-168 can be attributed to the activity of metabolite TQS-621.

The data demonstrate that oral administration of TQS-168 in solution provides higher TQS-168 and TQS-621 Cₘₐₓ and total drug exposure than is seen with two different suspension formulations.

We have also conducted a phase 1 human clinical trial and measured plasma concentrations of TQS-168 and active metabolite TQS-621 with three different formulations, and demonstrated that pharmacodynamically relevant plasma concentrations can be achieved with oral suspensions of several solid formulations of the API.

### 4.3. Methods of Reducing Neuroinflammation and/or Treating a Neurodegenerative Disease

### 4.3.1. Pharmacokinetic Dose Regimen

Accordingly, in a first aspect, methods are provided for reducing neuroinflammation and/or treating a neurodegenerative disease in a subject. The method comprises orally administering to a subject with neuroinflammation and/or a neurogenerative disease a pharmaceutical composition comprising the compound of formula (I)

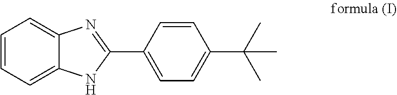 [SMILES: CC(C)(C)c1ccc(-c2nc3ccccc3[nH]2)cc1]

(TQS-168) (MW 250.3), or a pharmaceutically acceptable salt thereof, in amount that provides, following administration, (a) a mean peak blood or plasma TQS-168 concentration (Cₘₐₓ) of at least 50 nM (12.515 ng/mL). In certain embodiments, the amount provides (a) a mean peak blood or plasma TQS-168 concentration (Cₘₐₓ) of at least 50 nM (12.515 ng/mL) with (b) a mean time to Cₘₐₓ (Tₘₐₓ) of TQS-168 in blood or plasma of no more than 360 minutes. In some embodiments, Cₘₐₓ and Tₘₐₓ are measured in plasma.

In some embodiments, TQS-168 or salt thereof is administered in an amount that provides, following administration, a mean blood or plasma Cₘₐₓ of TQS-168 of at least 100 nM (25.03 ng/mL), 150 nM (37.545 ng/mL), 200 nM (50.06 ng/mL), 250 nM (62.575 ng/mL), 300 nM (75.09 ng/mL), 350 nM (87.605 ng/mL), 400 nM (100.12 ng/mL), 450 nM (112.635 ng/mL), 500 nM (125.15 ng/mL), 550 nM (137.665 ng/mL), 600 nM (150.18 ng/mL), 650 nM (162.695 ng/mL), 700 nM (175.21 ng/mL), 750 nM (187.725 ng/mL), 800 nM (200.24 ng/mL), 850 nM (212.755 ng/mL), 900 nM (225.27 ng/mL), 950 nM (237.785 ng/mL), 1 μM (250.3 ng/mL), 2 μM (500.6 ng/mL), 2.5 μM (625.75 ng/mL), 3 μM (750.9 ng/mL), 3.5 μM (876.05 ng/mL), 4 μM (1001.2 ng/mL), 4.5 μM (1126.35 ng/mL), 5 μM (1151.5 ng/mL), 5.5 μM (1376.65 ng/mL), 6 μM (1501.8 ng/mL), 6.5 μM (1626.95 ng/mL), 7 μM (1752.1 ng/mL), 7.5 μM (1877.25 ng/mL), 8 μM (2002.4 ng/mL), 8.5 μM (2127.55 ng/mL), 9 μM (2252.7 ng/mL), 9.5 μM (2377.85 ng/mL), 10 μM (2503 ng/mL), 10.5 μM (2628.15 ng/mL), 11 μM (2753.3 ng/mL), 11.5 μM (2878.45 ng/mL), 12 μM (3003.6 ng/mL), 12.5 μM (3128.75 ng/mL), 13 μM (3253.9 ng/mL), 13.5 μM (3379.05 ng/mL), 14 μM (3504.2 ng/mL), 14.5 μM (3629.35 ng/mL), 15 μM (3754.5 ng/mL), 15.5 μM (3879.65 ng/mL), 16 μM (4004.8 ng/mL), 16.5 μM (4129.95 ng/mL), 17 μM (4255.1 ng/mL), 17.5 μM (4380.25 ng/mL), 18 μM (4505.4 ng/mL), 18.5 μM (4630.55 ng/mL), 19 μM (4755.7 ng/mL), 19.5 μM (4880.85 ng/mL), 20 μM (5006 ng/mL), 20.5 μM (5131.15 ng/mL), 21 μM (5256.3 ng/mL), 21.5 μM (5381.45 ng/mL), 22 μM (5506.6 ng/mL), 22.5 μM (5631.75 ng/mL), 23 μM (5756.9 ng/mL), 23.5 μM (5882.05 ng/mL), 24 μM (6007.2 ng/mL), 24.5 μM (6132.35 ng/mL), or 25 μM (6275.5 ng/mL).

In certain embodiments, TQS-168 or salt thereof is administered in an amount that provides, following administration, a mean blood or plasma Cmax of TQS-168 of at least 3.5 μM (876.05 ng/mL), 4 μM (1001.2 ng/mL), 4.5 μM (1126.35 ng/mL), 5 μM (1151.5 ng/mL), 5.5 μM (1376.65 ng/mL), 6 μM (1501.8 ng/mL), 6.5 μM (1626.95 ng/mL), 7 μM (1752.1 ng/mL), 7.5 μM (18778.25 ng/mL), or 8 μM (2002.4 ng/mL).

In specific embodiments, TQS-168 or salt thereof is administered in an amount that provides, following administration, a mean blood or plasma Cmax of TQS-168 of at least 4 μM (1001.2 ng/mL), 4.5 μM (1126.35 ng/mL), 5 μM (1151.5 ng/mL), or 5.5 μM (1376.65 ng/mL).

In some embodiments, TQS-168 or salt thereof is administered in an amount that provides, following administration, a mean blood or plasma Cmax of TQS-168 between 2 μM (500.6 ng/mL) and 8 μM (2002.4 ng/mL), 2.5 μM (625.75 ng/mL) and 7.5 μM (1877.25 ng/mL), 3 μM (750.9 ng/mL) and 7 μM (1752.1 ng/mL), 3.5 μM (876.05 ng/mL), 6.5 μM (1626.95 ng/mL), or 4 μM (1001.2 ng/mL) to 6 μM (1501.8 ng/mL). In specific embodiments, TQS-168 or salt thereof is administered in an amount that provides, following administration, a mean blood or plasma Cmax of TQS-168 between 4 μM (1001.2 ng/mL) and 5 μM (1151.5 ng/mL).

In particular embodiments, TQS-168 or salt thereof is administered in an amount that provides, following administration, a mean blood or plasma Cmax of TQS-168 of about 4.5 μM (1126.35 ng/mL).

In some embodiments, TQS-168 or salt thereof is administered in an amount that provides, following administration, a mean blood or plasma Cmax of TQS-168 of at least 700 ng/mL, 750 ng/mL, 800 ng/mL, 850 ng/mL, 900 ng/mL, 950 ng/mL, 1000 ng/mL, 1500 ng/mL or 2000 ng/mL. In certain embodiments, TQS-168 or salt thereof is administered in an amount that provides, following administration, a mean blood or plasma Cmax of TQS-168 of at least 900 ng/mL, 950 ng/mL, 1000 ng/mL, 1500 ng/mL or 2000 ng/mL. In particular embodiments, TQS-168 or salt thereof is administered in an amount that provides, following administration, a mean blood or plasma Cmax of TQS-168 of at least 1000 ng/mL, 1100 ng/mL, 1200 ng/mL, 1300 ng/mL, 1400 ng/mL, 1500 ng/mL, 1600 ng/mL, 1700 ng/mL, 1800 ng/mL, 1900 ng/mL, or 2000 ng/mL. In certain embodiments, TQS-168 or salt thereof is administered in an amount that provides, following administration, a mean blood or plasma Cmax of TQS-168 between 900 ng/mL and 1300 ng/mL or 1000 ng/mL and 1200 ng/mL.

In some embodiments, TQS-168 or salt thereof is administered in an amount that provides, following administration, a mean blood or plasma Cmax of TQS-168 of about 1100 ng/mL.

In some embodiments, TQS-168 or salt thereof is administered in an amount that provides, following administration, a mean brain Cₘₐₓ of TQS-168 of at least 50 nM, 100 nM, 150 nM, 200 nM, 250 nM, 300 nM, 350 nM, 400 nM, 450 nM, 500 nM, 550 nM, 600 nM, 650 nM, 700 nM, 750 nM, 800 nM, 850 nM, 900 nM, 950 nM, 1 μM, 2 μM, 2.5 μM, 3 μM, 3.5 μM, 4 μM, 4.5 μM, 5 μM, 5.5 μM, 6 μM, 6.5 μM, 7 μM, 7.5 μM, 8 μM, 8.5 μM, 9 μM, 9.5 μM, 10 μM, 10.5 μM, 11 μM, 11.5 μM, 12 μM, 12.5 μM, 13 μM, 13.5 μM, 14 μM, 14.5 μM, 15 μM, 15.5 μM, 16 μM, 16.5 μM, 17 μM, 17.5 μM, 18 μM, 18.5 μM, 19 μM, 19.5 μM, 20 μM, 20.5 μM, 21 μM, 21.5 μM, 22 μM, 22.5 μM, 23 μM, 23.5 μM, 24 μM, 24.5 μM, or 25 μM.

In some embodiments, TQS-168 or salt thereof is administered in an amount that provides, following administration, a brain-to-plasma ratio of TQS-168 of 0.5-10. In certain embodiments, TQS-168 is administered in an amount that provides, following administration, a brain-to-plasma ratio of TQS-168 of 0.5, 0.75, 1.0, 1.25, 1.5, 1.75, 2.0, 2.25, 2.5, 2.75, 3.0, 3.25, 3.5, 3.75, 4.0, 4.25, 4.5, 4.75, or 5. In certain embodiments, TQS-168 is administered in an amount that provides, following administration, a brain-to-plasma ratio of TQS-168 of at least 1.0, at least 1.5, at least 2.0, at least 2.5, at least 3.0, at least 3.5, at least 4.0, at least 4.5 or at least 5.0.

In some embodiments, TQS-168 or salt thereof is administered in an amount that provides, following administration, an AUC₀₋ₜ of TQS-168, measured in plasma, of at least 2000 ng·hr/ml, 2500 ng·hr/ml, 3000 ng·hr/ml, 3500 ng·hr/ml, 4000 ng·hr/ml, 4500 ng·hr/ml, 5000 ng·hr/ml, 5500 ng·hr/ml, 6000 ng·hr/ml, 6500 ng·hr/ml, 7000 ng·hr/ml, 7500 ng·hr/ml, 8000 ng·hr/ml, 8500 ng·hr/ml, or 9000 ng·hr/ml. In certain embodiments, TQS-168 or salt thereof is administered in an amount that provides, following administration, an AUC₀₋ₜ of TQS-168 in plasma of at least 4000 ng·hr/ml, 4500 ng·hr/ml, 5000 ng·hr/ml, 5500 ng·hr/ml, 6000 ng·hr/ml, 6500 ng·hr/ml, or 7000 ng·hr/ml.

In certain embodiments, TQS-168 or salt thereof is administered in an amount that provides, following administration, an AUC₀₋ₜ of TQS-168 in plasma of between 4000 ng·hr/ml and 8000 ng·hr/ml. In particular embodiments, TQS-168 or salt thereof is administered in an amount that provides, following administration, an AUC₀₋ₜ of TQS-168 in plasma of between 5000 ng·hr/ml and 7000 ng·hr/ml. In specific embodiments, TQS-168 or salt thereof is administered in an amount that provides, following administration, an AUC₀₋ₜ of TQS-168 in plasma of about 6000 ng·hr/ml.

In some embodiments, TQS-168 or salt thereof is administered in an amount that provides, following administration, a mean Tₘₐₓ of TQS-168 in blood or plasma of no more than 360 minutes. In certain embodiments, TQS-168 or salt thereof is administered in a formulation that provides a mean Tₘₐₓ in blood or plasma of no more than 360 minutes, 350 minutes, 340 minutes, 330 minutes, 320 minutes, 310 minutes, 300 minutes, 290 minutes, 280 minutes, 270 minutes, 260 minutes, 250 minutes, 225 minutes, 200 minutes, or 180 minutes. In certain embodiments, TQS-168 or salt thereof is administered in a formulation that provides a mean Tₘₐₓ in blood or plasma of no more than 90 minutes, 60 minutes, or 45 minutes.

In particular embodiments, TQS-168 or salt thereof is administered in an amount that provides, following administration, a mean Tₘₐₓ of TQS-168 in blood or plasma of no more than 120 minutes, 90 minutes or 60 minutes. In specific embodiments, TQS-168 or salt thereof is administered in an amount that provides, following administration, a mean Tₘₐₓ of about 60 minutes.

In another aspect, methods are provided for treating neuroinflammation and/or treating a neurodegenerative disease in a subject. The method comprises orally administering to a subject with neuroinflammation and/or a neurogenerative disease a pharmaceutical composition comprising TQS-168, or a pharmaceutically acceptable salt thereof, in amount that provides following administration, (a) a mean peak plasma concentration (Cₘₐₓ) of the compound of Formula (II)

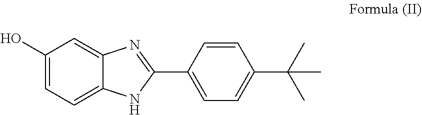 [SMILES: CC(C)(C)c1ccc(-c2nc3cc(O)ccc3[nH]2)cc1]

(TQS-621) (MW 266.3), of at least 50 nM, with (b) a mean time to Cₘₐₓ (Tₘₐₓ) of TQS-621 in plasma of no more than 360 minutes.

In some embodiments, TQS-168 or salt thereof is administered in an amount that provides, following administration, a mean blood or plasma Cₘₐₓ of TQS-621 of at least 100 nM, 150 nM, 200 nM, 250 nM, 300 nM, 350 nM, 400 nM, 450 nM, 500 nM, 550 nM, 600 nM, 650 nM, 700 nM, 750 nM, 800 nM, 850 nM, 900 nM, 950 nM, 1 μM, 2 μM, 2.5 μM, 3 μM, 3.5 μM, 4 μM, 4.5 μM, 5 μM, 5.5 μM, 6 μM, 6.5 μM, 7 μM, 7.5 μM, 8 μM, 8.5 μM, 9 μM, 9.5 μM, 10 μM, 10.5 μM, 11 μM, 11.5 μM, 12 μM, 12.5 μM, 13 μM, 13.5 μM, 14 μM, 14.5 μM, 15 μM, 15.5 μM, 16 μM, 16.5 μM, 17 μM, 17.5 μM, 18 μM, 18.5 μM, 19 μM, 19.5 μM, 20 μM, 20.5 μM, 21 μM, 21.5 μM, 22 μM, 22.5 μM, 23 μM, 23.5 μM, 24 μM, 24.5 μM, or 25 μM.

In some embodiments, TQS-168 or salt thereof is administered in an amount that provides, following administration, a mean blood or plasma Cₘₐₓ of TQS-621 of at least 75 ng/mL, 100 ng/mL, 125 ng/mL, 150 ng/mL, 175 ng/mL, 200 ng/mL, 225 ng/mL, 250 ng/mL, 250 ng/mL, 300 ng/mL, 350 ng/mL, 400 ng/mL, 450 ng/mL, 500 ng/mL, 550 ng/mL, or 600 ng/mL.

In some embodiments, TQS-168 or salt thereof is administered in an amount that provides, following administration, a mean blood or plasma Cₘₐₓ of TQS-621 of 100-700 ng/mL, 200-600 ng/mL, or 300-500 ng/mL.

In some embodiments, TQS-168 or salt thereof is administered in an amount that provides, following administration, a mean brain Cₘₐₓ of TQS-621 of at least 50 nM, 100 nM, 150 nM, 200 nM, 250 nM, 300 nM, 350 nM, 400 nM, 450 nM, 500 nM, 550 nM, 600 nM, 650 nM, 700 nM, 750 nM, 800 nM, 850 nM, 900 nM, 950 nM, 1 μM, 2 μM, 2.5 μM, 3 μM, 3.5 μM, 4 μM, 4.5 μM, 5 μM, 5.5 μM, 6 μM, 6.5 μM, 7 μM, 7.5 μM, 8 μM, 8.5 μM, 9 μM, 9.5 μM, 10 μM, 10.5 μM, 11 μM, 11.5 μM, 12 μM, 12.5 μM, 13 μM, 13.5 μM, 14 μM, 14.5 μM, 15 μM, 15.5 μM, 16 μM, 16.5 μM, 17 μM, 17.5 μM, 18 μM, 18.5 μM, 19 μM, 19.5 μM, 20 μM, 20.5 μM, 21 μM, 21.5 μM, 22 μM, 22.5 μM, 23 μM, 23.5 μM, 24 μM, 24.5 μM, or 25 μM.

In some embodiments, TQS-168 or salt thereof is administered in an amount that provides, following administration, a brain-to-plasma ratio of TQS-621 of 0.5-10. In certain embodiments, TQS-168 is administered in an amount that provides, following administration, a brain-to-plasma ratio of TQS-168 of 0.5, 0.75, 1.0, 1.25, 1.5, 1.75, 2.0, 2.25, 2.5, 2.75, 3.0, 3.25, 3.5, 3.75, 4.0, 4.25, 4.5, 4.75, or 5. In certain embodiments, TQS-168 is administered in an amount that provides, following administration, a brain-to-plasma ratio of TQS-168 of at least 1.0, at least 1.5, at least 2.0, at least 2.5, at least 3.0, at least 3.5, at least 4.0, at least 4.5 or at least 5.0.

In some embodiments, TQS-168 or salt thereof is administered in an amount that provides, following administration, a mean Tₘₐₓ of TQS-621 in blood or plasma of no more than 360 minutes. In certain embodiments, TQS-168 or salt thereof is administered in a formulation that provides a mean Tₘₐₓ in blood or plasma of no more than 360 minutes, 350 minutes, 340 minutes, 330 minutes, 320 minutes, 310 minutes, 300 minutes, 290 minutes, 280 minutes, 270 minutes, 260 minutes, 250 minutes, 225 minutes, 200 minutes, or 180 minutes. In certain embodiments, TQS-168 or salt thereof is administered in a formulation that provides a mean Tₘₐₓ in blood or plasma of no more than 90 minutes, 60 minutes, or 45 minutes.

In another aspect, methods of treating neuroinflammation and/or a neurodegenerative disease in a subject are provided. The methods comprise orally administering to a subject with neuroinflammation and/or a neurogenerative disease a pharmaceutical composition comprising TQS-168 or pharmaceutically acceptable salt thereof in an amount that provides, following administration, (a) a mean peak concentration (Cₘₐₓ) of TQS-168 in plasma of at least 50 nM, with (b) a mean time to Cₘₐₓ (Tₘₐₓ) of TQS-168 in plasma of no more than 360 minutes; and (c) a mean peak concentration (Cₘₐₓ) of (TQS-621) in plasma of at least 50 nM, with (d) a mean time to Cₘₐₓ (Tₘₐₓ) of TQS-621 in plasma of no more than 360 minutes.

In some embodiments, TQS-168 or salt thereof is administered in an amount that provides, following administration, a mean blood or plasma Cₘₐₓ of TQS-168 of at least 100 nM, 150 nM, 200 nM, 250 nM, 300 nM, 350 nM, 400 nM, 450 nM, 500 nM, 550 nM, 600 nM, 650 nM, 700 nM, 750 nM, 800 nM, 850 nM, 900 nM, 950 nM, 1 μM, 2 μM, 2.5 μM, 3 μM, 3.5 μM, 4 μM, 4.5 μM, 5 μM, 5.5 μM, 6 μM, 6.5 μM, 7 μM, 7.5 μM, 8 μM, 8.5 μM, 9 μM, 9.5 μM, 10 μM, 10.5 μM, 11 μM, 11.5 μM, 12 μM, 12.5 μM, 13 μM, 13.5 μM, 14 μM, 14.5 μM, 15 μM, 15.5 μM, 16 μM, 16.5 μM, 17 μM, 17.5 μM, 18 μM, 18.5 μM, 19 μM, 19.5 μM, 20 μM, 20.5 μM, 21 μM, 21.5 μM, 22 μM, 22.5 μM, 23 μM, 23.5 μM, 24 μM, 24.5 μM, or 25 μM.

In some embodiments, TQS-168 or salt thereof is administered in an amount that provides, following administration, a mean brain Cₘₐₓ of TQS-168 of at least 50 nM, 100 nM, 150 nM, 200 nM, 250 nM, 300 nM, 350 nM, 400 nM, 450 nM, 500 nM, 550 nM, 600 nM, 650 nM, 700 nM, 750 nM, 800 nM, 850 nM, 900 nM, 950 nM, 1 μM, 2 μM, 2.5 μM, 3 μM, 3.5 μM, 4 μM, 4.5 μM, 5 μM, 5.5 μM, 6 μM, 6.5 μM, 7 μM, 7.5 μM, 8 μM, 8.5 μM, 9 μM, 9.5 μM, 10 μM, 10.5 μM, 11 μM, 11.5 μM, 12 μM, 12.5 μM, 13 μM, 13.5 μM, 14 μM, 14.5 μM, 15 μM, 15.5 μM, 16 μM, 16.5 μM, 17 μM, 17.5 μM, 18 μM, 18.5 μM, 19 μM, 19.5 μM, 20 μM, 20.5 μM, 21 μM, 21.5 μM, 22 μM, 22.5 μM, 23 μM, 23.5 μM, 24 μM, 24.5 μM, or 25 μM.

In some embodiments, TQS-168 or salt thereof is administered in an amount that provides, following administration, a brain-to-plasma ratio of TQS-168 of 0.5-10. In certain embodiments, TQS-168 is administered in an amount that provides, following administration, a brain-to-plasma ratio of TQS-168 of 0.5, 0.75, 1.0, 1.25, 1.5, 1.75, 2.0, 2.25, 2.5, 2.75, 3.0, 3.25, 3.5, 3.75, 4.0, 4.25, 4.5, 4.75, or 5. In certain embodiments, TQS-168 is administered in an amount that provides, following administration, a brain-to-plasma ratio of TQS-168 of at least 1.0, at least 1.5, at least 2.0, at least 2.5, at least 3.0, at least 3.5, at least 4.0, at least 4.5 or at least 5.0.

In some embodiments, TQS-168 or salt thereof is administered in an amount that provides, following administration, a mean blood or plasma Cₘₐₓ of TQS-621 of at least 100 nM, 150 nM, 200 nM, 250 nM, 300 nM, 350 nM, 400 nM, 450 nM, 500 nM, 550 nM, 600 nM, 650 nM, 700 nM, 750 nM, 800 nM, 850 nM, 900 nM, 950 nM, 1 μM, 2 μM, 2.5 μM, 3 μM, 3.5 μM, 4 μM, 4.5 μM, 5 μM, 5.5 μM, 6 μM, 6.5 μM, 7 μM, 7.5 μM, 8 μM, 8.5 μM, 9 μM, 9.5 μM, 10 μM, 10.5 μM, 11 μM, 11.5 μM, 12 μM, 12.5 μM, 13 μM, 13.5 μM, 14 μM, 14.5 μM, 15 μM, 15.5 μM, 16 μM, 16.5 μM, 17 μM, 17.5 μM, 18 μM, 18.5 μM, 19 μM, 19.5 μM, 20 μM, 20.5 μM, 21 μM, 21.5 μM, 22 μM, 22.5 μM, 23 μM, 23.5 μM, 24 μM, 24.5 μM, or 25 μM.

In some embodiments, TQS-168 or salt thereof is administered in an amount that provides, following administration, a mean brain Cₘₐₓ of TQS-621 of at least 50 nM, 100 nM, 150 nM, 200 nM, 250 nM, 300 nM, 350 nM, 400 nM, 450 nM, 500 nM, 550 nM, 600 nM, 650 nM, 700 nM, 750 nM, 800 nM, 850 nM, 900 nM, 950 nM, 1 μM, 2 μM, 2.5 μM, 3 μM, 3.5 μM, 4 μM, 4.5 μM, 5 μM, 5.5 μM, 6 μM, 6.5 μM, 7 μM, 7.5 μM, 8 μM, 8.5 μM, 9 μM, 9.5 μM, 10 μM, 10.5 μM, 11 μM, 11.5 μM, 12 μM, 12.5 μM, 13 μM, 13.5 μM, 14 μM, 14.5 μM, 15 μM, 15.5 μM, 16 μM, 16.5 μM, 17 μM, 17.5 μM, 18 μM, 18.5 μM, 19 μM, 19.5 μM, 20 μM, 20.5 μM, 21 μM, 21.5 μM, 22 μM, 22.5 μM, 23 μM, 23.5 μM, 24 μM, 24.5 μM, or 25 μM.

In some embodiments, TQS-168 or salt thereof is administered in an amount that provides, following administration, a brain-to-plasma ratio of TQS-621 of 0.5-10. In certain embodiments, TQS-168 is administered in an amount that provides, following administration, a brain-to-plasma ratio of TQS-168 of 0.5, 0.75, 1.0, 1.25, 1.5, 1.75, 2.0, 2.25, 2.5, 2.75, 3.0, 3.25, 3.5, 3.75, 4.0, 4.25, 4.5, 4.75, or 5. In certain embodiments, TQS-168 is administered in an amount that provides, following administration, a brain-to-plasma ratio of TQS-168 of at least 1.0, at least 1.5, at least 2.0, at least 2.5, at least 3.0, at least 3.5, at least 4.0, at least 4.5 or at least 5.0.

Inhibition of inflammation in the periphery can provide benefit in treating neuroinflammation. For example, fingolimod, now approved for treating relapsing-remitting multiple sclerosis (MS), acts to reduce MS pathology by reducing lymphocyte egress from lymph nodes. Accordingly, in some embodiments, TQS-168 is administered in an amount that provides optimal concentrations of TQS-168 and metabolite TQS-621 in both peripheral and central compartments,

| | Plasma | Brain |
|---|---|---|
| parent | TQS-168 | TQS-168 |
| metabolite | TQS-621 | TQS-621 |

In various embodiments, TQS-168 is administered in an amount that provides optimal concentration ratios of one or more of:
TQS-168 plasma:TQS-168 brain
TQS-621 plasma:TQS-621 brain
TQS-168 plasma:TQS-621 plasma
TQS-168 brain:TQS-621 brain.

### 4.3.2. Oral Dose

In various embodiments, the daily oral dose of TQS-168 is at least 0.5 mg/kg. In various embodiments, the oral dose of TQS-168 is at least 1 mg/kg. In certain embodiments, the dose is at least 2 mg/kg, at least 3 mg/kg, at least 4 mg/kg, at least 5 mg/kg, at least 6 mg/kg, at least 7 mg/kg, at least 8 mg/kg, at least 9 mg/kg, or at least 10 mg/kg.

In various embodiments, the daily oral dose of TQS-168 is at least 10 mg/kg. In certain embodiments, the dose is at least 15 mg/kg, at least 20 mg/kg, at least 25 mg/kg, 30 mg/kg, at least 35 mg/kg, at least 40 mg/kg, at least 45 mg/kg, at least 50 mg/kg, at least 55 mg/kg, at least 60 mg/kg, at least 65 mg/kg, at least 70 mg/kg, at least 75 mg/kg, at least 80 mg/kg, at least 85 mg/kg, at least 90 mg/kg, at least 95 mg/kg, at least 100 mg/kg, at least 150 mg/kg, at least 175 mg/kg, or at least 200 mg/kg. In certain embodiments, the dose is 250 mg/kg, 300 mg/kg, 350 mg/kg, 400 mg/kg, 450 mg/kg, 500 mg/kg, 600 mg/kg, 650 mg/kg, 700 mg/kg, 750 mg/kg, 800 mg/kg, 850 mg/kg, 900 mg/kg, 950 mg/kg, or 1000 mg/kg. In certain embodiments, the oral dose is 0.5 mg/kg to 100 mg/kg per day. In certain embodiments, the oral dose is 2 mg/kg to 100 mg/kg per day. In certain embodiments, the oral dose is 25 mg/kg to 1000 mg/kg per day.

In various embodiments, the oral daily dose of TQS-168 is 25 mg/kg. In certain embodiments, the dose is at least 25 mg/kg. In certain embodiments, the dose is at least 50 mg/kg, at least 100 mg/kg, at least 150 mg/kg, at least 175 mg/kg, or at least 200 mg/kg. In certain embodiments, the dose is 250 mg/kg, 500 mg/kg, 750 mg/kg, or 1000 mg/kg. In certain embodiments, the oral dose is 25 mg/kg to 1,000 mg/kg per day.

In various embodiments, the daily oral dose is 10-5000 mg. In certain embodiments, the dose is 10 mg, 15 mg, 20 mg, 25 mg, 50 mg, 75 mg, 100 mg, 125 mg, 150 mg, 175 mg, 200 mg, 225 mg, 250 mg, 275 mg, 300 mg, 325 mg, 350 mg, 375 mg, 400 mg, 450 mg, 500 mg, 550 mg, 600 mg, 650 mg, 700 mg, 750 mg, 800 mg, 850 mg, 900 mg, 950 mg, or 1000 mg. In certain embodiments, the dose is 1500 mg, 2000 mg, 2500 mg, 3000 mg, 3500 mg, 4000 mg, 4500 mg, or 5000 mg.

In various embodiments, the daily dose is 25-2000 mg. In certain embodiments, the dose is 25 mg, 50 mg, 75 mg, 100 mg, 125 mg, 150 mg, 175 mg, 200 mg, 225 mg, 250 mg, 275 mg, 300 mg, 350 mg, 375 mg, 400 mg, 425 mg, 450 mg, 475 mg, 500 mg, 525 mg, 550 mg, 575 mg, 600 mg, 625 mg, 650 mg, 675 mg, 700 mg, 725 mg, 750 mg, 775 mg, 800 mg, 825 mg, 900 mg, 925 mg, 950 mg, 975 mg, or 1000 mg.

In certain embodiments, the daily oral dose is 200-800 mg. In particular embodiments, the daily oral dose is 350 mg, 360 mg, 370 mg, 380 mg, 390 mg, 400 mg, 410 mg, 420 mg, 430 mg, 440 mg, 450 mg, 460 mg, 470 mg, 480 mg, 490 mg, or 500 mg. In specific embodiments, the dose is 400 mg or 450 mg. In certain embodiments, the daily oral dose is 400 mg or 450 mg in a spray dried dispersion formulation.

### 4.3.3. Oral Dosage Form

In some embodiments, TQS-168 or salt thereof is administered in a suspension. In other embodiments, TQS-168 or salt thereof is administered in a solution. In some embodiments, TQS-168 or salt thereof is administered in a solid dosage form. In particular embodiments, the solid dosage form is a capsule. In particular embodiments, the solid dosage form is a tablet. In specific embodiments, TQS-168 is in a crystalline or amorphous form. In particular embodiments, TQS-168 is in amorphous form.

### 4.3.4. Patients

In various embodiments, the subject has neuroinflammation. In certain embodiments, the subject does not have a diagnosed neurodegenerative disease. In particular embodiments, the subject does not have a diagnosed neurodegenerative disease and is at least 40, 45, 50, 55, 60, 65, 70, or 75 years old. In particular embodiments, the subject does not have a diagnosed neurodegenerative disease but has one or more signs or symptoms of cognitive impairment. In specific embodiments, the subject has mild cognitive impairment (MCI).

In various embodiments, the subject has a neurodegenerative disease. In certain embodiments, the neurodegenerative disease is selected from a motor neuron disease, amyotrophic lateral sclerosis (ALS), Alzheimer's disease, vascular dementia, frontotemporal degeneration (frontotemporal dementia), dementia with Lewy bodies, Parkinson's disease, Huntington's disease, demyelinating disease, and multiple sclerosis (MS).

In particular embodiments, the subject has a motor neuron disease. In a specific embodiment, the subject has ALS. In particular embodiments, the subject has Alzheimer's disease. In particular embodiments, the subject has vascular dementia. In particular embodiments, the subject has frontotemporal dementia (FTD). In particular embodiments, the subject has dementia with Lewy bodies (Lewy body disease). In particular embodiments, the subject has Parkinson's disease. In particular embodiments, the subject has Huntington's disease. In yet additional embodiments, the subject has demyelinating disease. In one embodiment, the subject has MS.

### 4.3.5. Further Embodiments

Further embodiments are provided in the following numbered clauses.

1. A method of treating neuroinflammation and/or a neurodegenerative disease in a subject, the method comprising:
orally administering to a subject with neuroinflammation and/or a neurogenerative disease a pharmaceutical composition comprising the compound of formula (I)

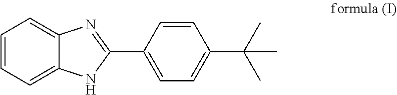 [SMILES: CC(C)(C)c1ccc(-c2nc3ccccc3[nH]2)cc1]

(TQS-168) or a pharmaceutically acceptable salt thereof, in amount that provides after administration,
(a) a mean peak concentration (Cₘₐₓ) of TQS-168 in plasma of at least 50 nM, with
(b) a mean time to Cₘₐₓ (Tₘₐₓ) of TQS-168 in plasma of no more than 360 minutes.
   2. The method of clause 1, wherein TQS-168 or salt thereof is administered in an amount that provides, following administration,
(a) a mean plasma TQS-168 Cₘₐₓ of at least 100 nM, 250 nM, or 500 nM.
   3. The method of clause 2, wherein TQS-168 or salt thereof is administered in an amount that provides, following administration,
(a) a mean plasma TQS-168 Cₘₐₓ of at least 750 nM.
   4. The method of clause 3, wherein TQS-168 or salt thereof is administered in an amount that provides, following administration,
(a) a mean plasma TQS-168 Cₘₐₓ of at least 1 μM.
   5. The method of clause 4, wherein TQS-168 or salt thereof is administered in an amount that provides, following administration,
(a) a mean plasma TQS-168 Cₘₐₓ of at least 5 μM.
   6. The method of clause 5, wherein TQS-168 or salt thereof is administered in an amount that provides, following administration,
(a) a mean plasma TQS-168 Cₘₐₓ of at least 7.5 μM.
   7. The method of clause 6, wherein TQS-168 or salt thereof is administered in an amount that provides, following administration,
(a) a mean plasma TQS-168 Cₘₐₓ of at least 10 μM.
   8. The method of any one of clauses 1-7, wherein TQS-168 or salt thereof is administered in a formulation that provides
(b) a mean plasma TQS-168 Tₘₐₓ of no more than 275 minutes.
   9. The method of clause 8, wherein TQS-168 or salt thereof is administered in a formulation that provides
(b) a mean plasma TQS-168 Tₘₐₓ of no more than 250 minutes.
   10. The method of clause 9, wherein TQS-168 or salt thereof is administered in a formulation that provides
(b) a mean plasma TQS-168 Tₘₐₓ of no more than 225 minutes.
   11. The method of clause 10, wherein TQS-168 or salt thereof is administered in a formulation that provides
(b) a mean plasma TQS-168 Tₘₐₓ of no more than 180 minutes, 90 minutes, 60 minutes or 45 minutes.
   12. A method of treating neuroinflammation and/or a neurodegenerative disease in a subject, the method comprising:
orally administering to a subject with neuroinflammation and/or a neurogenerative disease a pharmaceutical composition comprising the compound of formula (I)

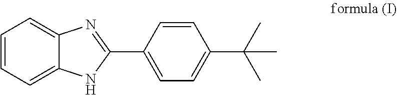 [SMILES: CC(C)(C)c1ccc(-c2nc3ccccc3[nH]2)cc1]

(TQS-168), or a pharmaceutically acceptable salt thereof, in amount that provides following administration,
(a) a mean peak plasma concentration (Cₘₐₓ) of the compound of Formula (II)

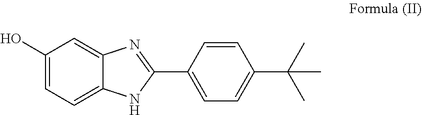 [SMILES: CC(C)(C)c1ccc(-c2nc3cc(O)ccc3[nH]2)cc1]

(TQS-621) of at least 50 nM, with
(b) a mean time to Cₘₐₓ (Tₘₐₓ) of TQS-621 in plasma of no more than 360 minutes.
   13. The method of clause 12, wherein TQS-168 or salt thereof is administered in an amount that provides, following administration,
(a) a mean plasma TQS-621 Cₘₐₓ of at least 100 nM, 250 nM, or 500 nM.
   14. The method of clause 13, wherein TQS-168 or salt thereof is administered in an amount that provides, following administration,
(a) a mean plasma TQS-621 Cₘₐₓ of at least 750 nM.
   15. The method of clause 14, wherein TQS-168 or salt thereof is administered in an amount that provides, following administration,
(a) a mean plasma TQS-621 Cₘₐₓ of at least 1 μM.
   16. The method of clause 15, wherein TQS-168 or salt thereof is administered in an amount that provides, following administration,
(a) a mean plasma TQS-621 Cₘₐₓ of at least 5 μM.
   17. The method of clause 16, wherein TQS-168 or salt thereof is administered in an amount that provides, following administration,
(a) a mean plasma TQS-621 Cₘₐₓ of at least 7.5 μM.
   18. The method of clause 17, wherein TQS-168 or salt thereof is administered in an amount that provides, following administration,
(a) a mean plasma TQS-621 Cₘₐₓ of at least 10 μM.
   19. The method of any one of clauses 12-18, wherein TQS-168 or salt thereof is administered in a formulation that provides
(b) a mean plasma TQS-621 Tₘₐₓ of no more than 275 minutes.
   20. The method of clause 19, wherein TQS-168 or salt thereof is administered in a formulation that provides
(b) a mean plasma Tₘₐₓ of TQS-168 of no more than 250 minutes.
   21. The method of clause 20, wherein TQS-168 or salt thereof is administered in a formulation that provides
(b) a mean plasma Tₘₐₓ of TQS-168 of no more than 225 minutes.
   22. The method of clause 21, wherein TQS-168 or salt thereof is administered in a formulation that provides
(b) a mean plasma Tₘₐₓ of TQS-168 of no more than 180 minutes, 90 minutes, 60 minutes or 45 minutes.
   23. A method of treating neuroinflammation and/or a neurodegenerative disease in a subject, the method comprising:
orally administering to a subject with neuroinflammation and/or a neurogenerative disease a pharmaceutical composition comprising the compound of formula (I)

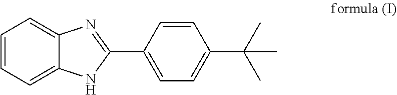 [SMILES: CC(C)(C)c1ccc(-c2nc3ccccc3[nH]2)cc1]

(TQS-168), or a pharmaceutically acceptable salt thereof, in amount that provides following administration,
(a) a mean peak concentration (Cₘₐₓ) of TQS-168 in plasma of at least 50 nM, with
(b) a mean time to Cₘₐₓ (Tₘₐₓ) of TQS-168 in plasma of no more than 360 minutes; and
(c) a mean peak concentration (Cₘₐₓ) of the compound of Formula (II)

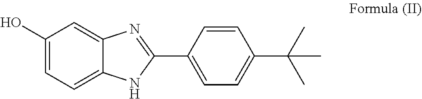 [SMILES: CC(C)(C)c1ccc(-c2nc3cc(O)ccc3[nH]2)cc1]

(TQS-621) in plasma of at least 50 nM, with
(d) a mean time to Cₘₐₓ (Tₘₐₓ) of TQS-621 in plasma of no more than 360 minutes.
   24. The method of any one of clauses 1-23, wherein TQS-168 or salt thereof is administered in a suspension.
   25. The method of any one of clauses 1-23, wherein TQS-168 or salt thereof is administered in a solution.
   26. The method of any one of clauses 1-23, wherein TQS-168 or salt thereof is administered in a solid dosage form.
   27. The method of clause 26, wherein the solid dosage form is a capsule.
   28. The method of clause 26, wherein the solid dosage form is a tablet.
   29. The method of any one of clauses 1-24 or 26-28, wherein TQS-168 is in a crystalline or amorphous form.
   30. The method of any one of clauses 1-24 or 26-28, wherein TQS-168 is in amorphous form.
   31. The method of any one of clauses 1-30, wherein the subject has a neurodegenerative disease selected from a motor neuron disease, amyotrophic lateral sclerosis (ALS), Alzheimer's disease, vascular dementia, frontotemporal degeneration (frontotemporal dementia), dementia with Lewy bodies, Parkinson's disease, Huntington's disease, demyelinating disease, and multiple sclerosis (MS).
   32. The method of clause 31, wherein the subject has a motor neuron disease.
   33. The method of clause 31, wherein the subject has ALS.
   34. The method of clause 31, wherein the subject has Alzheimer's disease.
   35. The method of clause 31, wherein the subject has vascular dementia.
   36. The method of clause 31, wherein the subject has frontotemporal dementia (FTD).
   37. The method of clause 31, wherein the subject has dementia with Lewy bodies (Lewy body disease).
   38. The method of clause 31, wherein the subject has Parkinson's disease.
   39. The method of clause 31, wherein the subject has Huntington's disease.
   40. The method of clause 31, wherein the subject has demyelinating disease.
   41. The method of clause 31, wherein the subject has MS.
   42. The method of any one of clauses 1 to 41, wherein the dose of TQS-168 is at least 0.5 mg/kg.
   43. The method of clause 42, wherein the dose of TQS-168 is at least 2 mg/kg.
   44. The method of clause 43, wherein the dose of TQS-168 is at least 4 mg/kg.
   45. The method of clause 44, wherein the dose of TQS-168 is at least 8 mg/kg.
   46. The method of clause 45, wherein the dose of TQS-168 is at least 12 mg/kg.
   47. The method of clause 46, wherein the dose of TQS-168 is at least 14 mg/kg.
   48. The method of clause 47, wherein the dose of TQS-168 is at least 16 mg/kg.
   49. The method of clause 48, wherein the dose is 2 mg/kg.
   50. The method of clause 49, wherein the dose is 4 mg/kg.
   51. The method of clause 50, wherein the dose is 8 mg/kg.

### 5. EXPERIMENTAL EXAMPLES

Below are examples of specific embodiments for carrying out the present invention. The examples are offered for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed for.

The practice of the present invention will employ, unless otherwise indicated, conventional methods of protein chemistry, biochemistry, recombinant DNA techniques and pharmacology, within the skill of the art. Such techniques are explained fully in the literature.

### 5.1. Example 1—TQS-168 Induces PGC-1α Protein Expression in a Murine Myeloid Cell Line In Vitro at 20 μM

Frozen BV2 murine microglia cells were thawed and propagated in complete media (RPMI, 10% heat-inactivated FBS, 1% L-glutamine, 1% Pen-Strep) until the growth rate reached log phase.

For protein expression analysis, DMSO (1:1000 final dilution) or TQS-168 in DMSO (1:1000 final dilution) at 20 μM was added to cell cultures. After 24 hours stimulation, supernatant was discarded and cell lysis buffer (Cell Signal) was added to adherent cells to extract protein. Total protein was quantified and normalized across all samples by BCA assays.

Two replicates of untreated cultures and three replicates of TQS-168-treated cultures were investigated, with each replicate containing lysates from 5,000,000 BV2 myeloid cells.

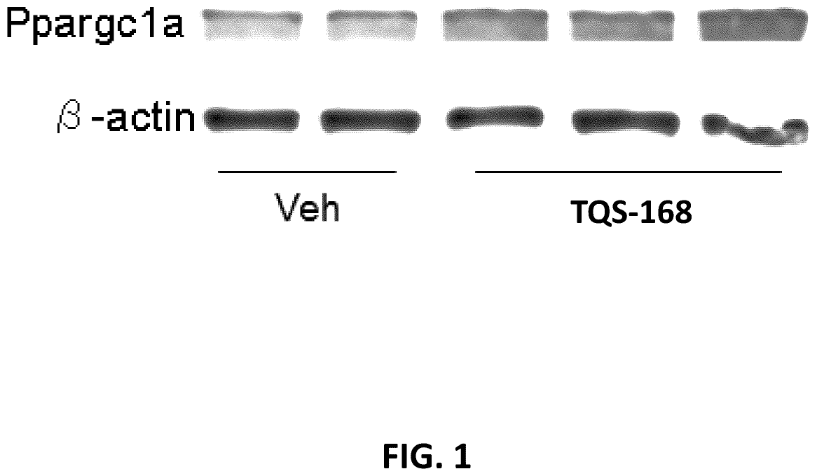

PGC1α expression was detected by Western Blot using an anti-PGC-1α antibody (SC13067, Santa Cruz Biotechnology, 1:500 dilution). Anti-R-actin antibody (SC8432, Santa Cruz Biotechnology, 1:2000 dilution) was used to quantify beta-actin, a housekeeping gene whose expression level is not known to be affected by TQS-168. A representative Western blot is shown in FIG. 1.

As shown, TQS-168 induces PGC-1α protein expression in murine BV2 microglia cells at 20 μM in vitro.

### 5.2. Example 2—TQS-168 Induces PGC-1α Protein Expression in a Murine Myeloid Cell Line In Vitro at Concentrations of 0.7 to 20 μM

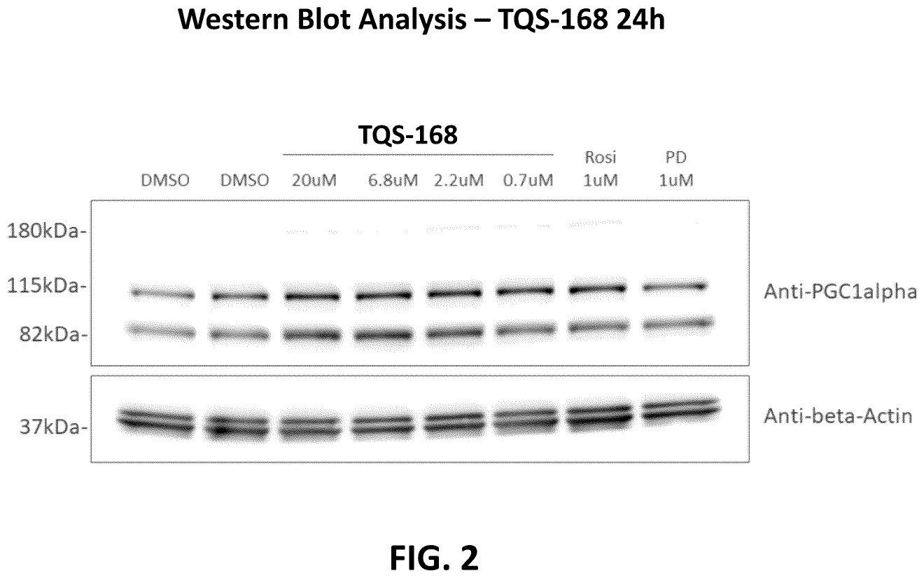

For protein expression analysis, DMSO (1:1000 final dilution) or TQS-168 in DMSO (1:1000 final dilution) at 20 μM, 6.8 μM, 2.2 μM, and 0.7 μM were added to BV2 cell cultures. After 24 hours' stimulation, supernatant was discarded and cell lysis buffer (Cell Signal) was added to adherent cells to extract protein. Total protein was quantified and normalized across all samples by BCA assays. PGC-1α was subsequently detected by Western Blot using an anti-PGC-1α antibody (SC13067, Santa Cruz Biotechnology, 1:500 dilution). β-actin was detected with an anti-β-actin antibody (SC8432, Santa Cruz Biotechnology, 1:2000 dilution). Results are shown in FIG. 2. All procedures were performed with standard molecular biology protocols for protein expression studies.

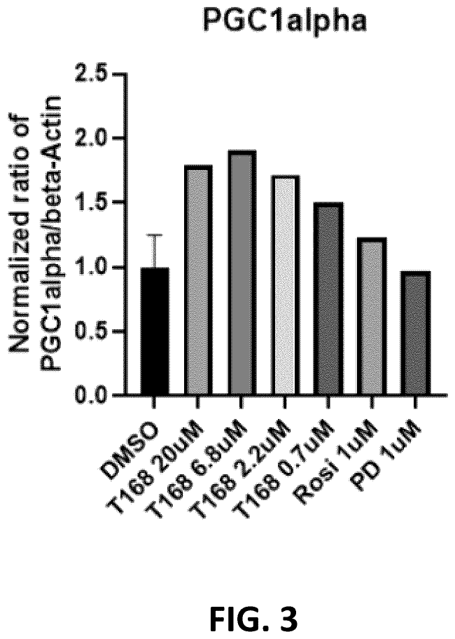

FIG. 3 is a bar graph quantifying protein expression levels measured from scans of the Western Blot shown in FIG. 2. PD refers to PD169316, a p38 MAPK inhibitor.

FIGS. 2 and 3 demonstrate that TQS-168 induces PGC-1α protein expression in murine BV2 cells in vitro at concentrations ranging from 0.7 μM to 20 μM.

### 5.3. Example 3—TQS-168 Inhibits Inflammatory Cytokine Secretion from Lps-Stimulated BV2 Cells In Vitro at Concentrations Ranging From 1 μM to 20 μM (CBA Assay)

Lipopolysaccharide (LPS) is a natural ligand of the TLR4/CD14 complex, which is highly expressed on myeloid cells

LPS was used to induce cytokine secretion from BV-2 cells. Cells were incubated with TQS-168 at various concentrations to assess whether TQS-168 could suppress the LPS-promoted release of various cytokines from in vitro cell cultures. Cytokine secretion was measured by cytometric bead array (CBA) fluorescence-activated cell sorting (FACS).

Briefly, a vial of BV2 frozen stock (1 million cells per ml in complete medium) was thawed into 10 mL of complete medium per vial for a total of 4 vials. The vials were then centrifuged at 1800 rpm for 3 minutes to wash away freezing media. The four vials were then pooled into 25 mL of complete medium. TQS-168 was prepared in DMSO.

Cells were incubated for 24 hours in the presence of medium (negative control), LPS (positive control), DMSO+LPS (positive control, controlling additionally for presence of DMSO in the TQS-168 stock solution), or LPS+TQS-168 at final concentrations of 1 μM, 5 μM, 10 μM, and 20 μM.

After the well plates were incubated overnight for 24 hours, they were centrifuged at 1800 rpm for 5 minutes. Next, 150 μl from each well was transferred to a new set of plates, of which 50 μl was used for CBA. The plates were then centrifuged with the cells, followed by addition of 100 μL of DAPI (50 ml of PBS+10 μL of DAPI stock at 1:5000 dilution). The plates were then incubated in the dark for 5 minutes, followed by addition of 100 μL of PBS and centrifuged at 1800 rpm for 5 minutes. Finally, the plates were resuspended in 200 μL of PBS and tested.

Multiplexed cytometric bead array (CBA) assay was performed according to standard techniques to detect presence in the cell culture medium of secreted murine TNFα, IL-6, IFNγ, IL-12, monocyte chemoattractant protein 1 (MCP-1), and IL-10.

### Conclusion

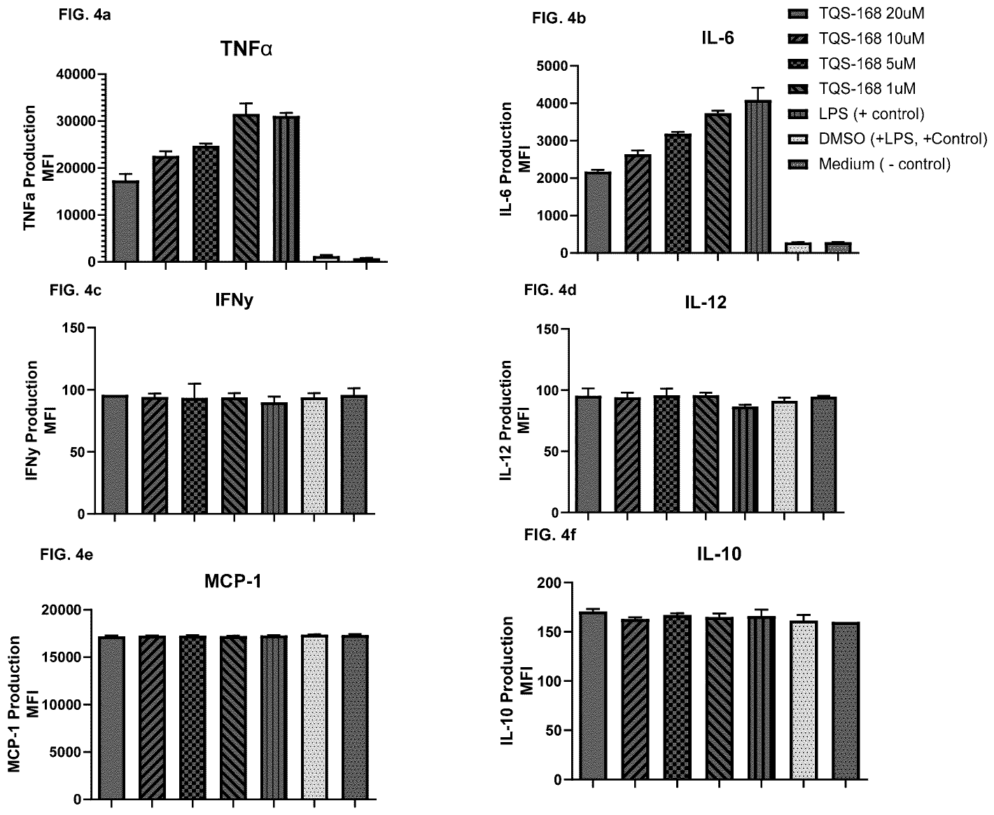

As shown in FIG. 4, BV2 cells treated with 100 ng/ml LPS displayed strong release of TNFα and IL-6 pro-inflammatory cytokines, while secretion of INF-γ, IL-10, IL-12 and MCP-1 were not significantly induced. TQS-168 was able to reduce the LPS-induced secretion of TNFα and IL-6 from BV2 cells at concentrations ranging from 1 μM-20 μM. Reductions of both TNFα (FIG. 4a) and IL-6 (FIG. 4b) by TQS-168 were concentration-dependent.

### 5.4. Example 4—TQS-168 Inhibits LPS-Induced TNFα Secretion from BV2 Myeloid Cells In Vitro at 5 μM and 20 μM (CBA Assay)

A total of 4-11 replicates of BV2 cultures in different conditions were investigated. Frozen BV2 microglia cell line was thawed and propagated in complete media (RPMI, 10% heat inactivated FBS, 1% L-glutamine, 1% Pen-Strep) until growth rate reached log phase.

For TNFα stimulation, cells were stimulated with 100 ng/mL of LPS for 24 hours. DMSO (1:1000 final dilution) or TQS-168 in DMSO (1:1000 final dilution) at either 5 μM or 20 μM were added to cell cultures. After 24 hours stimulation, supernatant was collected for cytokine analysis with CBA assays (BD Biosciences) per manufacturer's protocols. TNFα express was normalized to DMSO treated condition.

ANOVA was used for statistical analysis with significant threshold at p-value<0.05.

### Conclusion

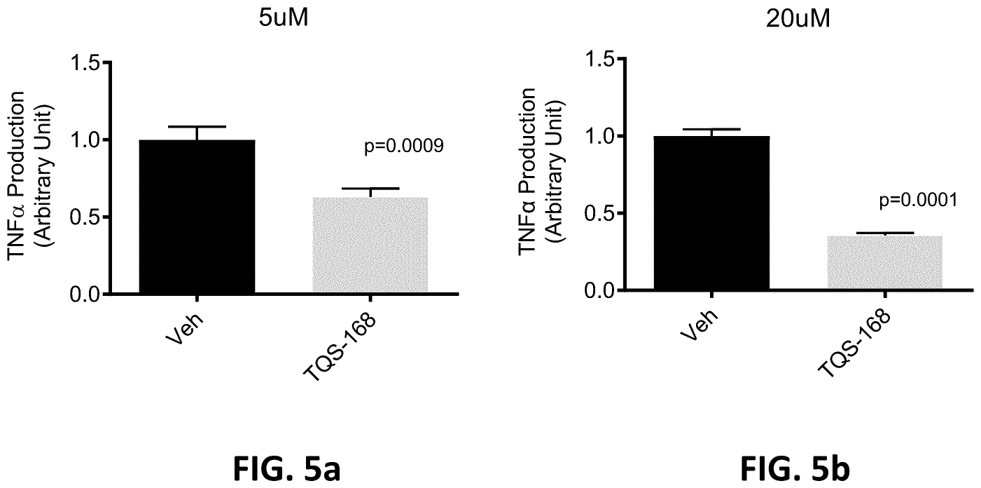

As shown in FIGS. 5a and 5b, TQS-168 at 5 μM and 20 μM, respectively, can suppress TNFα production by LPS-stimulated BV-2 myeloid cells, as compared to control.

### 5.5. Example 5—TQS-168 Inhibits TNFα Release by BV2 Cells Stimulated With LPS In Vitro at Concentrations of 0.3 μM to 10 μM (ELISA)

### TNFα ELISA Procedure

First, 100 μL of supernatant was removed from Cell Plate and transferred to a Dilution Plate, which was then centrifuged at 216×g for 10 minutes to remove particulates. The dilution plate was either assayed immediately or aliquots were taken and stored at ≤−20° C.; repeated freeze-thaw cycles were avoided.

Next, a standard curve was prepared by first pipetting 900 ul of Calibrator Diluent RD5K into the 700 μg/mL tube, followed by 200 μl of the appropriate calibrator diluent in the remaining tubes. The stock solution was used to produce a dilution. The resulting tubes were then thoroughly mixed. The Mouse TNFα Standard (700 μg/mL) served as the high standard, and the Calibrator Diluent RD5T served as the zero standard at 0 pg/ml.

Assay Diluent RD1-63 (50 uL) was then added to the center of each well and mixed before and during its use. Then, 50 ul of either standard, control, or sample was added to the center of each well, and covered with adhesive strip. The plate was then mixed for 1 minute and incubated for 2 hours at room temperature.

Each well was then aspirated and washed by filling each well with Wash Buffer (400 ul) five times. After the last wash, the remaining Wash Buffer was removed by aspirating or decanting. TNFα IL-6 conjugate (100 ul) was then added to each well, covered with adhesive strip, incubated for 2 hours at room temperature, and washed and/or aspirated five times.

Substrate solution (100 uL) was then added to each well, incubated in the dark for 30 minutes at room temperature, followed by addition of 100 μl of Stop Solution and mixed. The optical density of each well was determined within 30 minutes by using a microplate reader set to 450 nm.

### Measurement Settings and Parameters and Data Processing

Plates were read on a Spectrostar Nano machine with built-in MARS data analysis at 450 nM and 570 nM.

### Optimization Parameters

Titration of LPS stimulation was performed to optimize the assay dynamic range.

For TNFα ELISA, the BV-2 cells were very responsive to low concentrations of LPS. A concentration range of 0.1 ng/mL to 1,000 ng/mL was tested. 0.3 ng/ml LPS produced enough TNFα release (8-10 fold above background) from these cells after 22 hours stimulation without saturating the linear range of the ELISA detection system. If higher LPS concentrations are used for stimulating BV-2 cells, a sample solution is advised to stay within the linear range of the detection system. The current TNFα protocol uses 10,000 cells per well in 96 well plate. A cell count titration can optimize S/B ratio. Miniaturization from 96 well to 384 well is also feasible with this assay.

### Conclusion

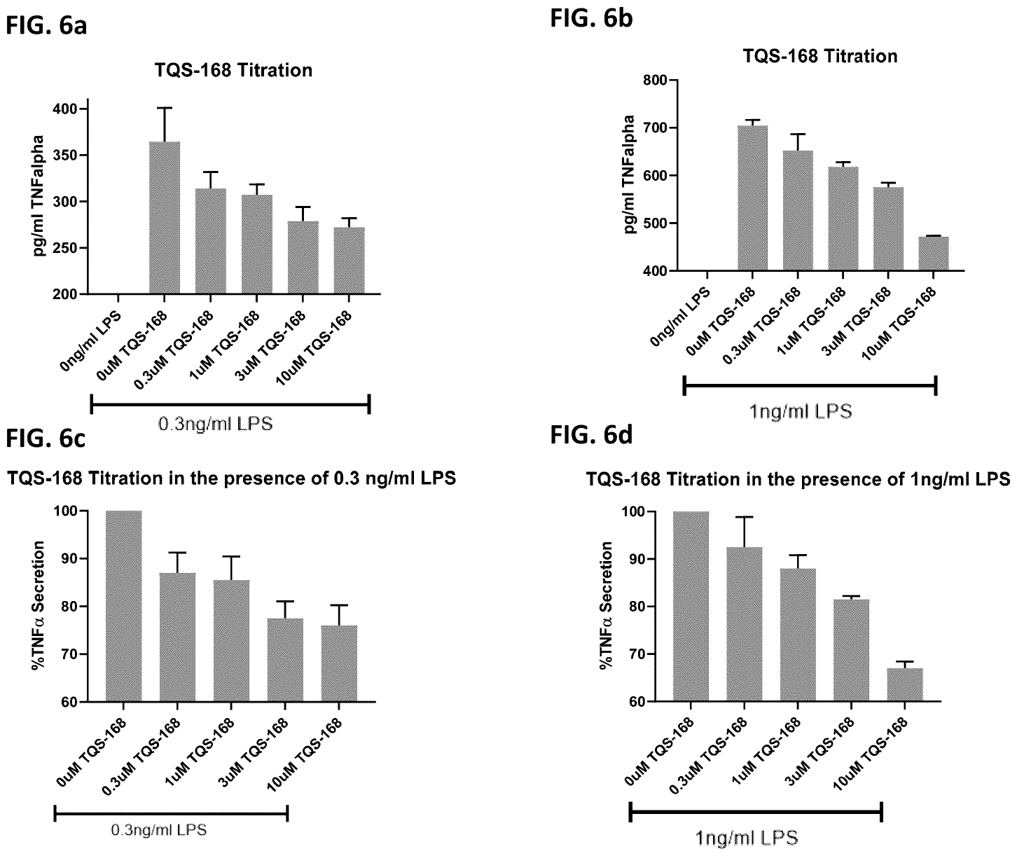

FIGS. 6a-6d show the dose response of TQS-168-mediated decrease of pro-inflammatory cytokine TNFα release from microglia BV2 cells using ELISA at 24 hours. FIG. 6a and FIG. 6c show the absolute (FIG. 6a) and relative (FIG. 6c) inhibition of TNFα secretion by BV2 cells stimulated with 0.3 ng/mL of LPS. FIG. 6b and FIG. 6d show the absolute (FIG. 6b) and relative (FIG. 6d) inhibition of TNFα secretion by BV2 cells stimulated with 1 ng/mL of LPS.

In BV-2 cells treated with 0.3 ng/mL of LPS, administration of TQS-168 suppressed TNFα production in a concentration-dependent manner, with up to about 25% suppression observed for cells administered with 10 μM of TQS-168.

Similarly, in BV-2 cells treated with 1 ng/mL of LPS, administration of TQS-168 suppressed TNFα production in a concentration-dependent manner, with up to about 35% inhibition for cells administered with 10 μM of TQS-168.

### 5.6. Example 6—TQS-168 Inhibits LPS-Induced TNF-α Production by Primary Human Myeloid Cells

### Study Methodology

Peripheral blood mononuclear cells (PBMC) from 4 different healthy volunteers were used in this study. Fresh blood samples were collected at Stanford Blood Center and processed for PBMC isolation with Ficoll gradient. PBMC samples were stored in liquid nitrogen at −80° C. for subsequent analysis of TNF-α production.

For TNF-α stimulation, frozen PBMC samples were thawed and rested at 37° C. before cells were stimulated with 100 ng/ml of LPS for 24 hours. DMSO (1:1000 final dilution) or TQS-168 in DMSO (1:1000 final dilution) at various concentrations was added to cell cultures of LPS-stimulated PBMC to evaluate the effects of T-168 on TNF-α production by human primary myeloid cells. After 24 hours stimulation, supernatant samples from various conditions were collected and TNF-α concentrations in the supernatants analyzed with cytometric bead array (CBA) assay per protocols from BD Biosciences. TNF-α was quantified by median fluorescent intensity reading (MFI). Samples were analyzed directly after staining with LSRII flow cytometer.

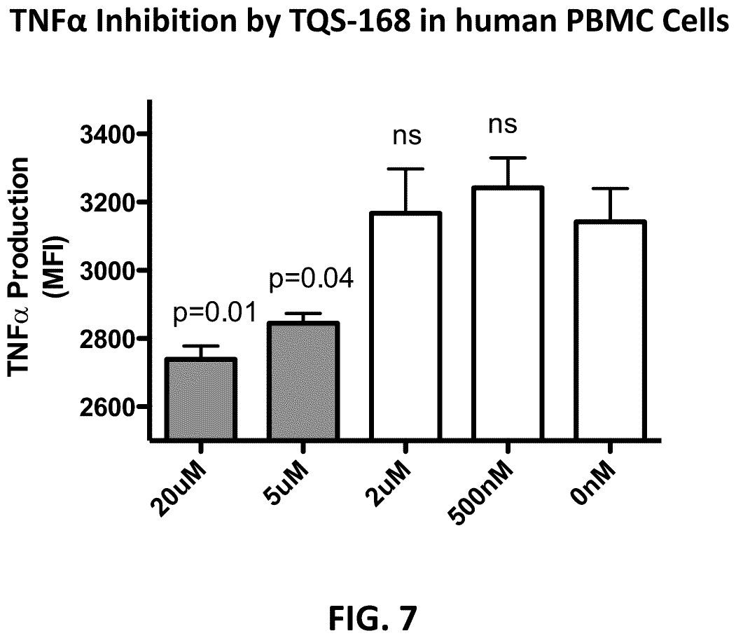

As shown in FIG. 7, in human PMBC cells treated with 100 ng/mL LPS, administration of TQS-168 at 5 μM and 20 μM led to statistically significant suppression of TNFα production as compared to no treatment.

### 5.7. Example 7—TQS-168 Tissue Concentrations after Single Oral Dose in Mice

### Study Methodology

In a first experiment, a total of 3-4 male C57BL6/J mice were administered a single dose of TQS-168 by oral gavage at 25 mg/kg. TQS-168 was prepared for oral gavage as a suspension with 0.5% methylcellulose in PBS.

Tissues were collected at various time points after dosing and processed for LC-MS analyses of TQS-168 concentrations. The tissues analyzed include plasma, brain, and liver. Animals were perfused thoroughly with 20 mL ice cold PBS before brain and liver collection to remove contaminating blood. Data are shown in Table 1 (TQS-168 concentrations in ng/mL).

TABLE 1

| Time (minutes) | 0.0 | 15.0 | 60.0 | 120.0 | 240.0 | 480.0 | 1440.0 | 2880.0 |
|---|---|---|---|---|---|---|---|---|
| Plasma 1 | 0.5 | 18.8 | 107.0 | 110.0 | 46.8 | 6.0 | 0.0 | 0.0 |
| Plasma 2 | 0.1 | 12.6 | 47.0 | 43.8 | 175.0 | 2.7 | 0.0 | 0.0 |
| Plasma 3 | 0.0 | 18.6 | 57.4 | 88.6 | 3.4 | 1.8 | 0.0 | 0.0 |
| Plasma 4 | | 14.6 | 86.0 | 131.0 | 14.0 | 10.9 | 0.0 | 0.0 |
| Plasma Average | 0.2 | 16.2 | 74.4 | 93.4 | 59.8 | 5.3 | 0.0 | 0.0 |
| Brain 1 | 22.8 | 35.2 | 630.0 | 242.5 | 10.6 | 18.2 | 55.5 | 0.7 |
| Brain 2 | 4.2 | 40.2 | 253.0 | 269.5 | 399.0 | 34.8 | 36.3 | 0.0 |
| Brain 3 | 10.7 | 21.6 | 755.0 | 282.0 | 143.0 | 20.9 | 8.5 | 3.0 |
| Brain 4 | | 42.0 | 530.0 | 150.5 | 188.0 | 5.3 | 10.5 | 2.5 |
| Brain Average | 12.6 | 34.7 | 542.0 | 236.1 | 185.2 | 19.8 | 27.7 | 1.5 |
| Liver 1 | 2.4 | 268.0 | 1060.0 | 305.5 | 60.5 | 431.5 | 4.0 | 0.0 |
| Liver 2 | 0.4 | 496.0 | 2725.0 | 2080.0 | 1520.0 | 50.5 | 2.6 | 1.0 |
| Liver 3 | 0.7 | 575.0 | 1940.0 | 1070.0 | 216.5 | 37.6 | 0.8 | 0.0 |
| Liver 3 | | 431.5 | 2175.0 | 1725.0 | 680.0 | 147.5 | 8.7 | 0.0 |
| Liver Average | 1.2 | 442.6 | 1975.0 | 1295.1 | 619.3 | 166.8 | 4.0 | 0.3 |

### Conclusion

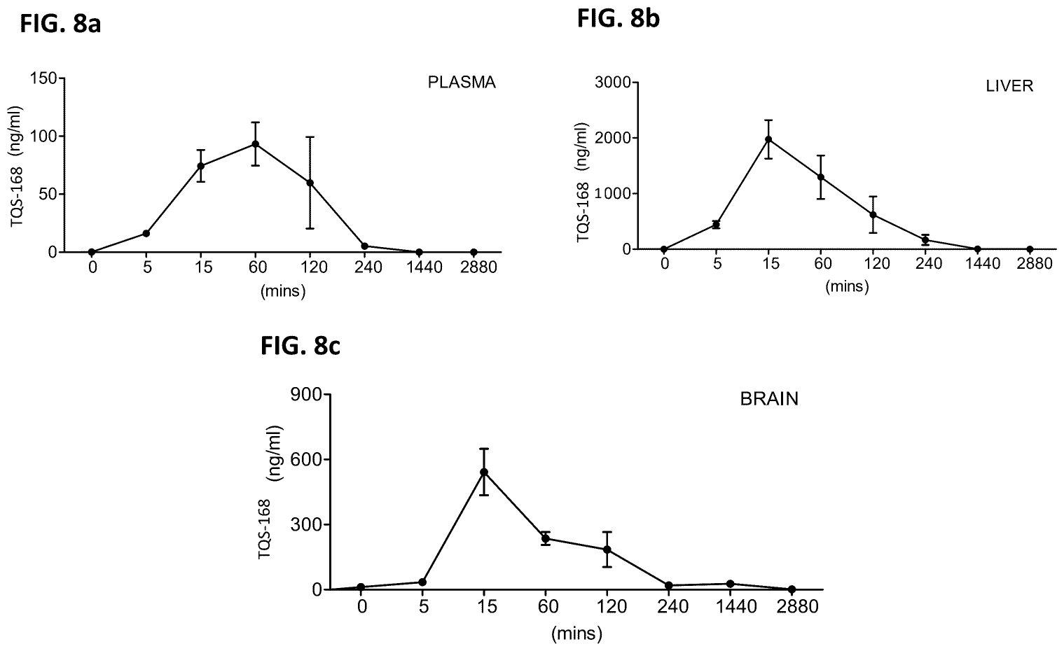

Average plasma Cₘₐₓ was 93.4 ng/ml, or 0.37 μM. Average brain Cₘₐₓ was 542.0 ng/ml, or 2.16 μM. Average concentration of TQS-168 (ng/ml) is graphed in FIG. 8 for plasma (FIG. 8a), liver homogenate (FIG. 8b), and brain homogenate (FIG. 8c).

In a second experiment, mice were administered 50 mg/kg TQS-168 by oral gavage. For Group 1, TQS-168 was prepared at a concentration of 5.0 mg/mL in a suspension with 0.500 methylcellulose in PBS. For Group 2, TQS-168 was prepared at a concentration of 5.0 mg/mL as a solution in 10 polyethylene glycol (PEG) 400|30 Kleptose (Roquete)|60% water.

Plasma exposures for Group 1 mice (ng/mL) are presented in Table 2 and for Group 2 mice in Table 3.

TABLE 2
Group 1 - TQS-168 p.o 50 mg/kg

| | Calculated Concentration (ng/mL) for T168 | | | | | |
|---|---|---|---|---|---|---|
| Time (hr) | Mouse #1 | Mouse #2 | Mouse #3 | Mean | SD | CV (%) |
| 0.25 | 586 | 887 | 485 | 653 | 209 | 32.0 |
| 0.5 | 848 | 1380 | 619 | 949 | 390 | 41.1 |
| 1 | 1280 | 1310 | 704 | 1098 | 342 | 31.1 |
| 2 | 839 | 1050 | 440 | 776 | 310 | 39.9 |
| 4 | 435 | 479 | 391 | 435 | 44 | 10.1 |
| 8 | 254 | 603 | 480 | 446 | 177 | 39.7 |
| 12 | 18.0 | 450 | 93.9 | 187 | 231 | 123.1 |
| 24 | BLOQ | BLOQ | BLOQ | NA | NA | NA |

TABLE 3
Group 2 - TQS-168 p.o. 50 mg/kg

| | Calculated Concentration (ng/ml) for T168 | | | | | |
|---|---|---|---|---|---|---|
| Time (br) | Mouse #4 | Mouse #5 | Mouse #6 | Mean | SD | CV (%) |
| 0.25 | 656 | 874 | 1170 | 900 | 258 | 28.7 |
| 0.5 | 1950 | 2070 | 1860 | 1960 | 105 | 5.4 |
| 1 | 1520 | 2390 | 2070 | 1993 | 440 | 22.1 |
| 2 | 1810 | 1830 | 1630 | 1757 | 110 | 6.3 |
| 4 | 1330 | 1330 | 949 | 1203 | 220 | 18.3 |
| 8 | 374 | 329 | 579 | 427 | 133 | 31.2 |
| 12 | 185 | 110 | 187 | 161 | 44 | 27.3 |
| 24 | BLOQ | BLOQ | BLOQ | NA | NA | NA |

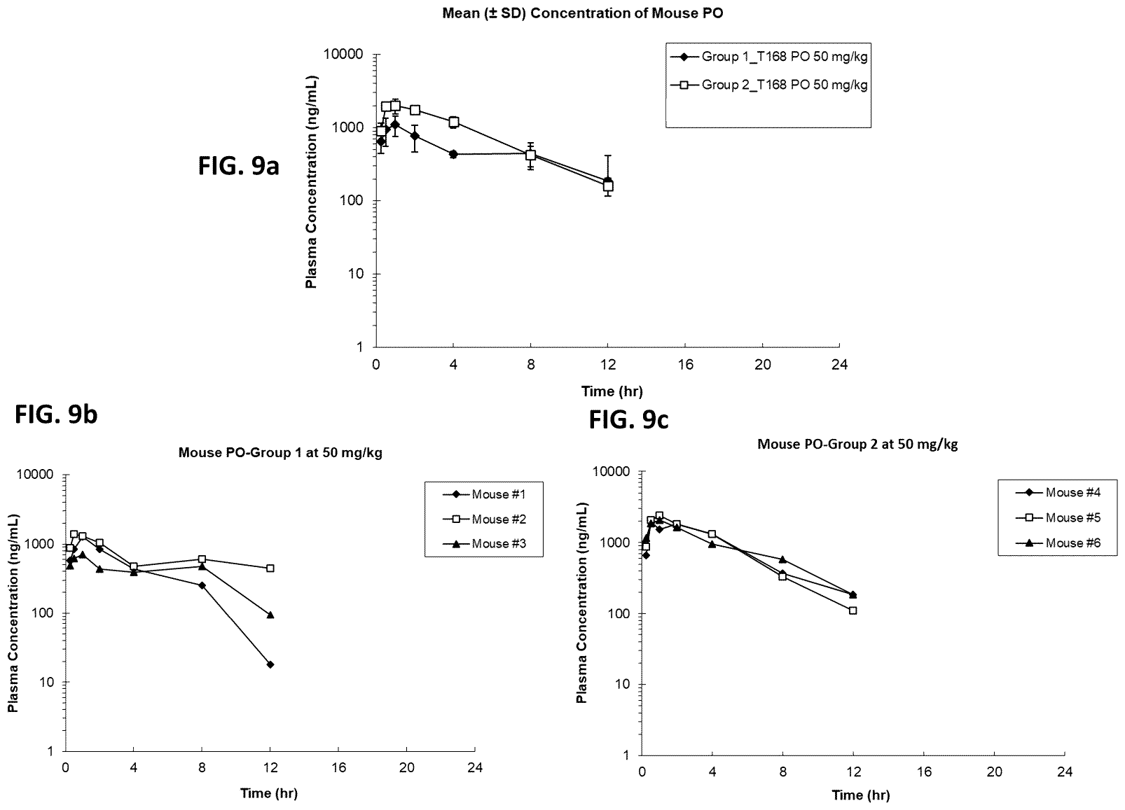

FIGS. 9a-9c show the plasma concentration of TQS-168 over time in Group 1 mice and Group 2 mice.

### Conclusion

Following a single oral dose of 50 mg/kg, twice the dose that had previously been demonstrated to suppress myeloid-mediated inflammation and reduce disease severity in animal models of neurodegenerative diseases, including Parkinson's disease, Alzheimer's disease, and amyotrophic lateral sclerosis (ALS), Cₘₐₓ in plasma was 2137 ng/mL, or 8.54 μM, with a time to Cₘₐₓ (Tₘₐₓ) of 50 mins.

### 5.8. Example 8—TQS-168 Plasma and Brain Concentrations after Single Oral Dose in Mice

Mice were administered a single oral dose of 45 mg/kg TQS-168. TQS-168 was prepared at a concentration of 5.0 mg/mL as a solution in 10% polyethylene glycol (PEG) 400|130% Kleptose (Roquette) 60% water.

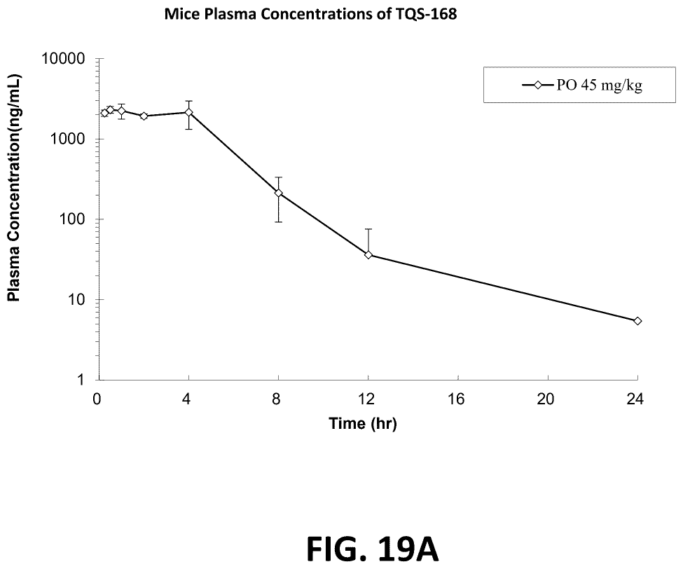

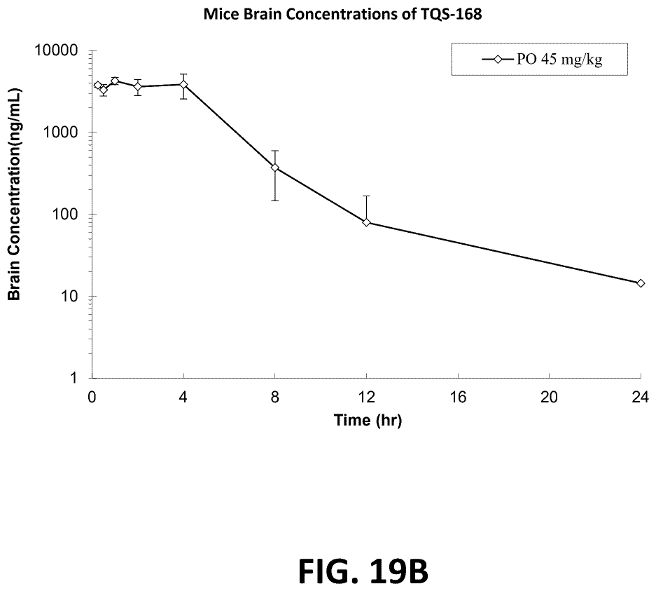

Concentrations in plasma and in brain were measured at various timepoints and various pharmacokinetic parameters were calculated, as shown below in Tables 4 and 5 and in FIGS. 19A and 19B.

TABLE 4
Plasma

| | | | | | | | | Last time point | Time points | |
|---|---|---|---|---|---|---|---|---|---|---|
| t_{1/2} | tₘₐₓ | Cₘₐₓ | AUCₗₐₛₜ | AUC_{Inf} | AUC_{Extr} | MRT_{Inf} | AUC_{Inf}/D | for AUCₗₐₛₜ | for t_{1/2} | |
| (hr) | (hr) | (ng/mL) | (hr*ng/mL) | (hr*ng/mL) | (%) | (hr) | (hr*kg*ng/mL/mg) | (hr) | (hr) | Rsq |
| 3.26 | 0.5 | 2313 | 13569 | 13595 | 0.188 | 3.35 | 302 | 24 | 8, 12, 24 | 0.932 |

TABLE 5
Brain

| | | | | | | | | Last time point | Time points | |
|---|---|---|---|---|---|---|---|---|---|---|
| t_{1/2} | tₘₐₓ | Cₘₐₓ | AUCₗₐₛₜ | AUC_{Inf} | AUC_{Extr} | MRT_{Inf} | AUC_{Inf}/D | for AUCₗₐₛₜ | for t_{1/2} | |
| (hr) | (hr) | (ng/g) | (hr*ng/g) | (hr*ng/g) | (%) | (hr) | (hr*kg*ng/g/mg) | (hr) | (hr) | Rsq |
| 3.66 | 1 | 4262 | 24592 | 24668 | 0.308 | 3.45 | 548 | 24 | 8, 12, 24 | 0.938 |

Brain-to-plasma Cₘₐₓ ratio of TQS-168 was 1.842. Brain-to-plasma AUC ratio of TQS-168 was 1.814.

### 5.9. Example 9—Metabolite TQS-621 Inhibits LPS-Stimulated Release of Pro-Inflammatory Cytokines from Human PBMCs

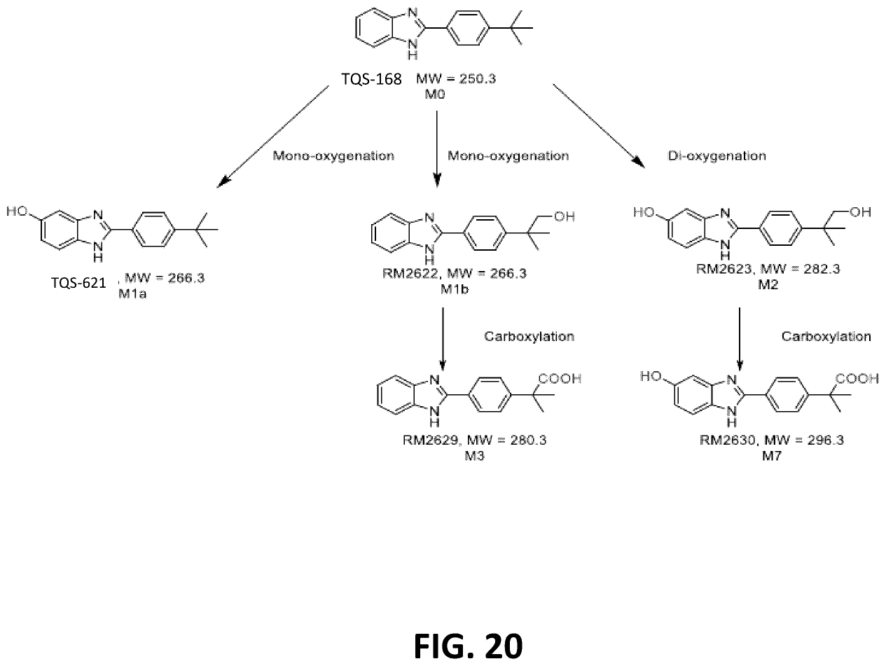

The principal metabolites from liver metabolism of TQS-168 following oral administration have been described. See Sun et al., Rapid Commun. Mass Spectrom. 32:480-488 (2018), incorporated herein by reference. We synthesized the phase 1 metabolites from liver metabolism of TQS-168 illustrated in FIG. 20 and tested them for the ability to inhibit LPS-stimulated secretion of pro-inflammatory cytokines in vitro.

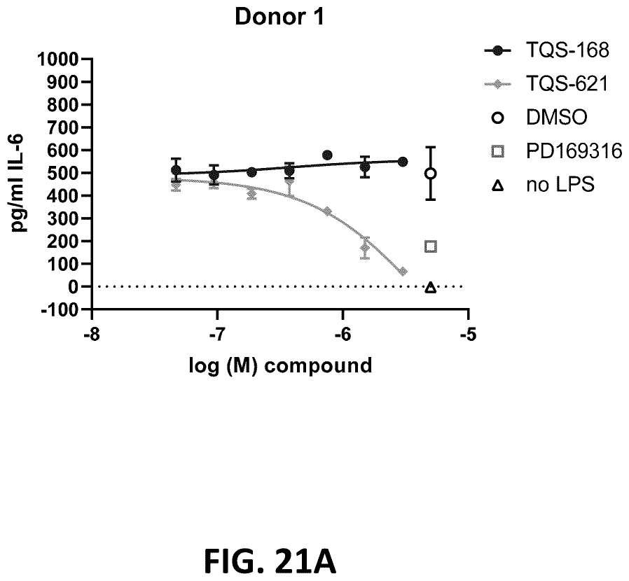

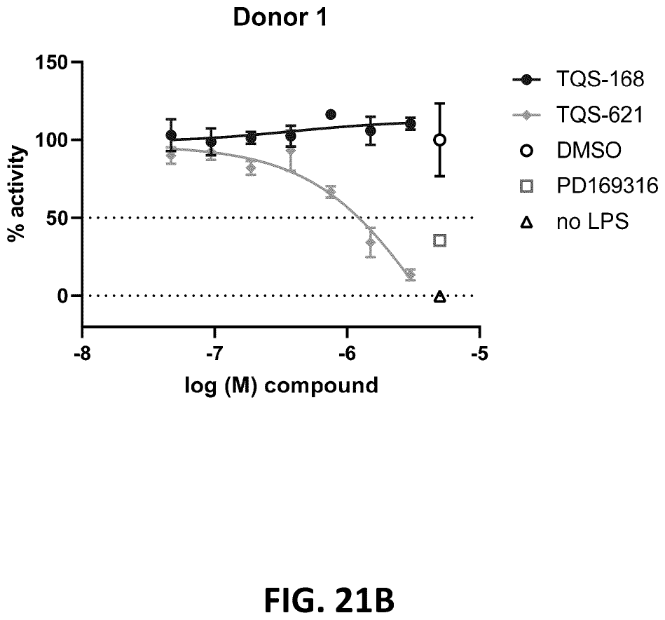

Briefly, 20,000 human PBMC were aliquoted into each well. LPS was added at 1 ng/mL and the cells incubated for 24 h in the presence of LPS and TQS-168 or one of its metabolites. Readouts were human IL-6 ELISA and human TNFα ELISA. For assay, supernatants were diluted in culture medium. The supernatant dilution for the IL-6 ELISA was 1:8, and the 1:2 for the human TNFα ELISA. Results are shown in FIGS. 21A and 21B.

FIG. 21A shows absolute inhibition of LPS-stimulated IL-6 secretion by TQS-168 metabolite TQS-621 from PBMCs obtained from a first donor. FIG. 21B shows the relative inhibition of IL-6 inhibition expressed as percentage activity. The structure of TQS-621 is shown below as Formula II:

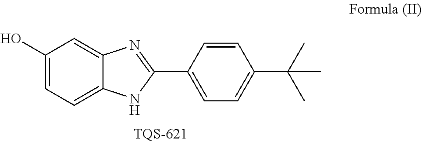 [SMILES: CC(C)(C)c1ccc(-c2nc3cc(O)ccc3[nH]2)cc1]

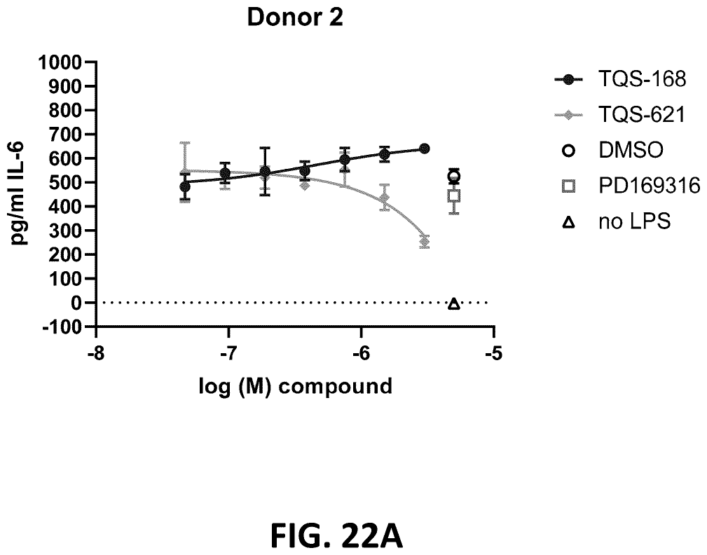

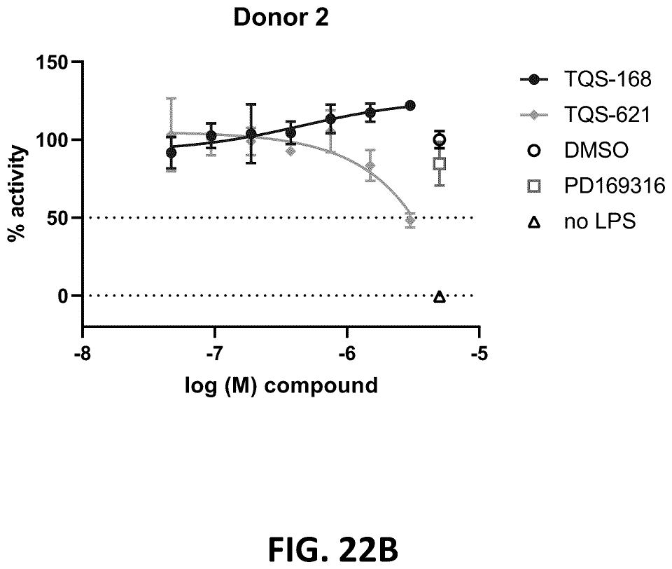

FIG. 22A shows absolute inhibition of LPS-stimulated IL-6 secretion by TQS-168 metabolite TQS-621 from PBMCs obtained from a second donor. FIG. 22B shows the relative inhibition of IL-6 inhibition expressed as percentage activity.

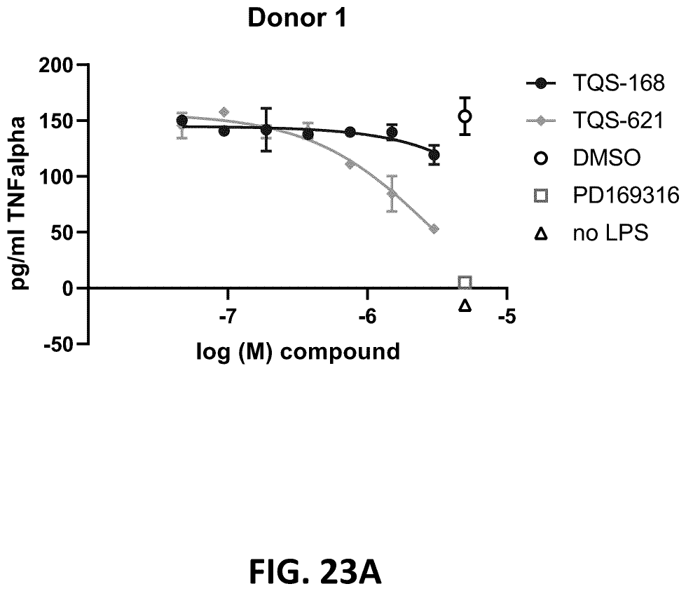

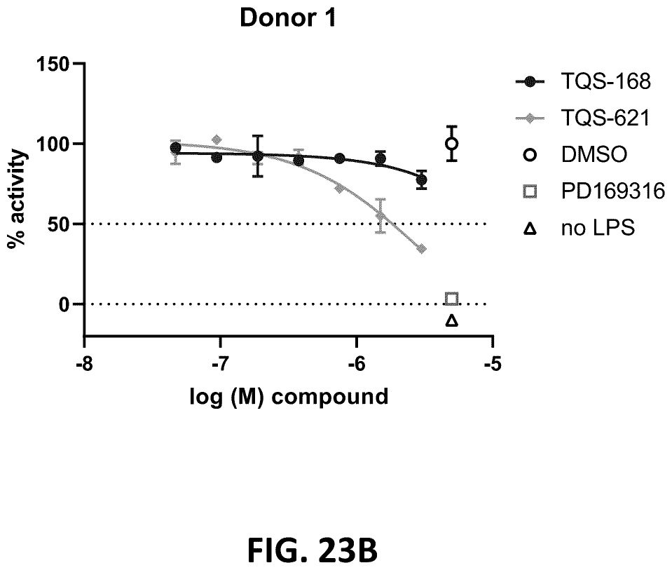

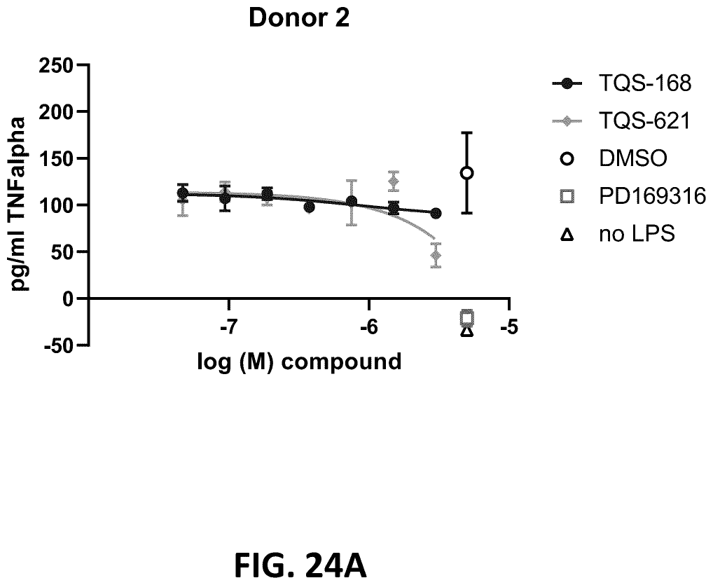

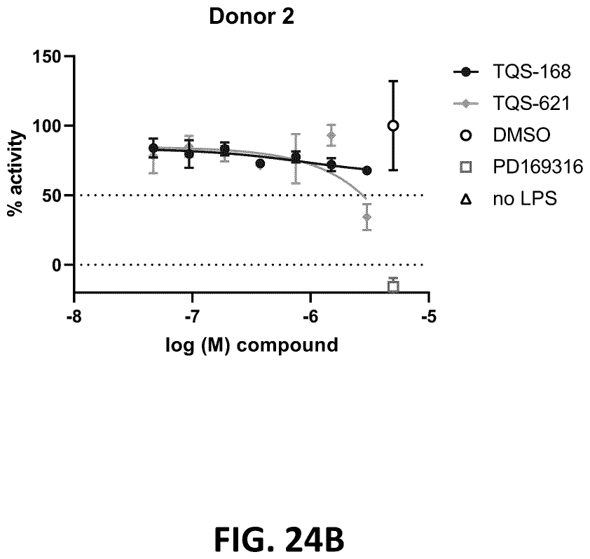

FIG. 23A shows absolute inhibition of LPS-stimulated TNFα secretion by TQS-168 and metabolite TQS-621 from PBMCs of the first donor. FIG. 23B shows the relative inhibition of TNFα inhibition expressed as percentage activity. FIG. 24A shows the absolute inhibition of LPS-stimulated TNFα secretion by TQS-168 and metabolite TQS-621 from PBMCs obtained from the second donor. FIG. 24B shows relative inhibition of TNFα inhibition expressed as percentage activity.

The data show that phase 1 metabolite TQS-621 is a potent inhibitor of LPS-stimulated IL-6 and TNFα secretion from human PBMCs, demonstrating that at least some of the therapeutic effect observed after oral administration of TQS-168 can likely be attributed to activity of active metabolite TQS-621.

### 5.10. Example 10—Plasma Concentrations of TQS-168 and Active Metabolite TQS-621 after a Single Oral Dose of TQS-168 in Mice

C57BL/6 male mice were administered a single dose of TQS-168 at 50 mg/kg using one of three formulations: Group 1-5.0 mg/ml suspension in 0.5% methylcellulose in PBS; Group 2-5.0 mg/ml solution in PEG400 (Fluka) 10%|Kleptose (Roquette) 30%|sterile water 60%; Group 3-5.0 mg/ml suspension in Labrafil M 1944 CS (Gattefosse) 30%|Masine CC (Gattefosse) 5%|Miglyol 812N 10%|sterile water 55%.

Plasma concentrations of TQS-168 and TQS-621 were measured overtime.

Table 6 presents the TQS-168 plasma concentration data for Group 1 mice; Table 7 presents the TQS-168 plasma concentration data for Group 2 mice; Table 8 provides the TQS-168 plasma concentration data for Group 3 mice.

TABLE 6
Group 1: TQS-168 plasma concentrations

| | t_{1/2} | tₘₐₓ | Cₘₐₓ | AUCₗₐₛₜ | AUC_{Inf} |
|---|---|---|---|---|---|
| Animal | (hr) | (hr) | (ng/mL) | (hr*ng/mL) | (hr*ng/mL) |
| Mouse #1 | 1.74 | 1 | 1280 | 5040 | 5085 |
| Mouse #2 | 9.47 | 0.5 | 1380 | 8046 | 14196 |
| Mouse #3 | 1.70 | 1 | 704 | 4822 | 5052 |
| N | 3 | 3 | 3 | 3 | 3 |
| Mean | 4.30 | 0.833 | 1121 | 5969 | 8111 |
| SD | 4.48 | 0.289 | 365 | 1802 | 5270 |
| CV % | 104.0 | 34.6 | 32.5 | 30.2 | 65.0 |

TABLE 7
Group 2: TQS-168 plasma concentrations

| | t_{1/2} | tₘₐₓ | Cₘₐₓ | AUCₗₐₛₜ | AUC_{Inf} |
|---|---|---|---|---|---|
| Animal | (hr) | (hr) | (ng/mL) | (hr*ng/mL) | (hr*ng/mL) |
| Mouse #4 | 2.81 | 0.5 | 1950 | 10606 | 11357 |
| Mouse #5 | 2.22 | 1 | 2390 | 11058 | 11411 |
| Mouse #6 | 3.41 | 1 | 2070 | 10525 | 11446 |
| N | 3 | 3 | 3 | 3 | 3 |
| Mean | 2.82 | 0.833 | 2137 | 10730 | 11404 |
| SD | 0.59 | 0.289 | 227 | 287 | 45 |
| CV % | 21.1 | 34.6 | 10.6 | 2.7 | 0.4 |

TABLE 8
Group 3: TQS-168 plasma concentrations

| | t_{1/2} | tₘₐₓ | Cₘₐₓ | AUCₗₐₛₜ | AUC_{Inf} |
|---|---|---|---|---|---|
| Animal | (hr) | (hr) | (ng/mL) | (hr*ng/mL) | (hr*ng/mL) |
| Mouse #7 | 1.47 | 1 | 611 | 4109 | 4247 |
| Mouse #8 | 2.23 | 0.5 | 752 | 3924 | 4336 |
| Mouse #9 | 1.43 | 8 | 420 | 3753 | 3877 |
| N | 3 | 3 | 3 | 3 | 3 |
| Mean | 1.71 | 3.17 | 594 | 3929 | 4153 |
| SD | 0.45 | 4.19 | 167 | 178 | 243 |
| CV % | 26.6 | 132.4 | 28.0 | 4.5 | 5.86 |

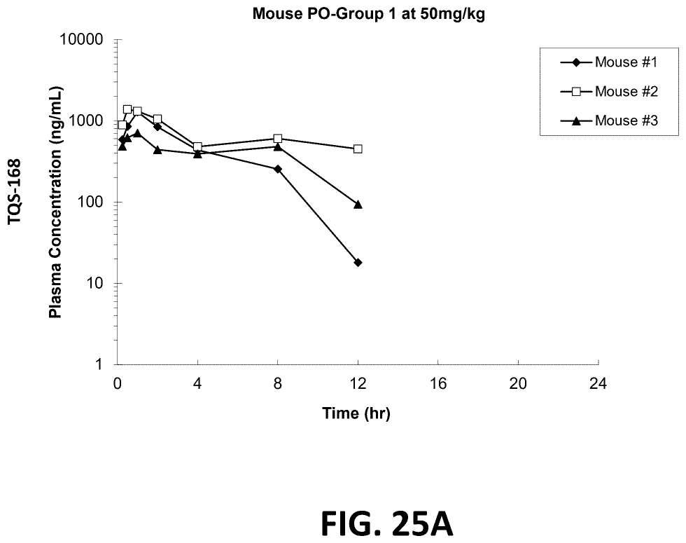

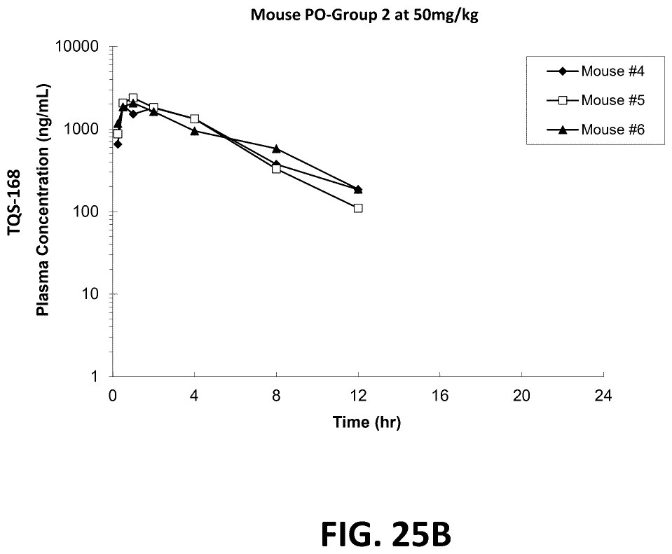

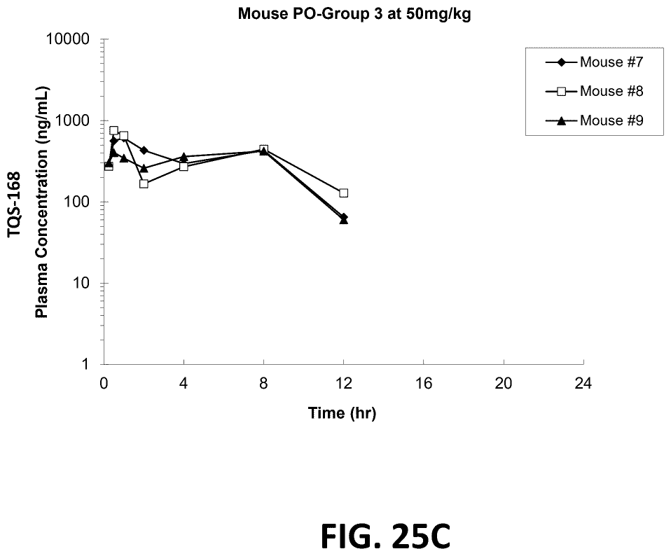

The solution formulation, Group 2, provided higher TQS-168 Cₘₐₓ (1950 ng/mL) (7.9 μM) than the suspension formulations, Group 1 (1121 ng/mL) (4.5 μM) and Group 3 (611 ng/mL) (2.4 μM) and higher exposure (AUC_{inf}=11404 hr*ng/mL) than the suspension formulations, Group 1 (8111) and Group 3 (4153). Results are plotted in FIG. 25A (Group 1), 25B (Group 2), and 25C (Group 3).

Table 9 presents the TQS-621 plasma concentration data for Group 1 mice; Table 10 presents the TQS-621 plasma concentration data for Group 2 mice; Table 11 provides the TQS-168 plasma concentration data for Group 3 mice.

TABLE 9
Group 1: TQS-621 plasma concentrations

| | | | | | | Cₘₐₓ Ratio | AUCₗₐₛₜ Ratio |
|---|---|---|---|---|---|---|---|
| | t_{1/2} | tₘₐₓ | Cₘₐₓ | AUCₗₐₛₜ | AUC_{Inf} | (Metabolite/ | (Metabolite/ |
| Animal | (hr) | (hr) | (ng/mL) | (hr*ng/mL) | (hr*ng/mL) | Parent) | Parent) |
| Mouse #1 | 1.88 | 1 | 239 | 1148 | 1163 | 0.187 | 0.228 |
| Mouse #2 | 80.8 | 2 | 309 | 2355 | 22064 | 0.224 | 0.293 |
| Mouse #3 | 2.08 | 1 | 156 | 1267 | 1379 | 0.222 | 0.263 |
| N | 3 | 3 | 3 | 3 | 3 | 3 | 3 |
| Mean | 28.3 | 1.33 | 235 | 1590 | 8202 | 0.211 | 0.261 |
| SD | 45.5 | 0.58 | 77 | 665 | 12005 | 0.021 | 0.033 |
| CV % | 161.1 | 43.3 | 32.6 | 41.8 | 146.4 | 9.9 | 12.5 |

TABLE 10
Group 2: TQS-621 plasma concentrations

| | | | | | | Cₘₐₓ Ratio | AUCₗₐₛₜ Ratio |
|---|---|---|---|---|---|---|---|
| | t_{1/2} | tₘₐₓ | Cₘₐₓ | AUCₗₐₛₜ | AUC_{Inf} | (Metabolite/ | (Metabolite/ |
| Animal | (hr) | (hr) | (ng/mL) | (hr*ng/mL) | (hr*ng/mL) | Parent) | Parent) |
| Mouse #4 | 2.92 | 2 | 487 | 2884 | 3114 | 0.250 | 0.272 |
| Mouse #5 | 2.17 | 1 | 483 | 2862 | 2962 | 0.202 | 0.259 |
| Mouse #6 | 3.61 | 2 | 378 | 2513 | 2817 | 0.183 | 0.239 |
| N | 3 | 3 | 3 | 3 | 3 | 3 | 3 |
| Mean | 2.90 | 1.67 | 449 | 2753 | 2964 | 0.211 | 0.256 |
| SD | 0.72 | 0.58 | 62 | 208 | 149 | 0.035 | 0.017 |
| CV % | 24.9 | 34.6 | 13.8 | 7.6 | 5.0 | 16.3 | 6.5 |

TABLE 11
Group 3: TQS-621 plasma concentrations

| | | | | | | Cₘₐₓ Ratio | AUCₗₐₛₜ Ratio |
|---|---|---|---|---|---|---|---|
| | t_{1/2} | tₘₐₓ | Cₘₐₓ | AUCₗₐₛₜ | AUC_{Inf} | (Metabolite/ | (Metabolite/ |
| Animal | (hr) | (hr) | (ng/mL) | (hr*ng/mL) | (hr*ng/mL) | Parent) | Parent) |
| Mouse #7 | 3.43 | 1 | 98.9 | 564 | 612 | 0.162 | 0.137 |
| Mouse #8 | 2.93 | 1 | 117 | 532 | 621 | 0.156 | 0.136 |
| Mouse #9 | 1.38 | 8 | 58.7 | 504 | 519 | 0.140 | 0.134 |
| N | 3 | 3 | 3 | 3 | 3 | 3 | 3 |
| Mean | 2.58 | 3.33 | 92 | 533 | 584 | 0.152 | 0.136 |
| SD | 1.07 | 4.04 | 30 | 30 | 56 | 0.011 | 0.002 |
| CV % | 41.4 | 121.2 | 32.6 | 5.6 | 9.6 | 7.5 | 1.1 |

The TQS-621 plasma Cₘₐₓ of the Group 2 mice which received the solution formulation, was 449 ng/mL, providing a Cₘₐₓ ratio of metabolite TQS-621 to parent TQS-168 of 0.152, and an AUCₗₐₛₜ ratio of 0.136.

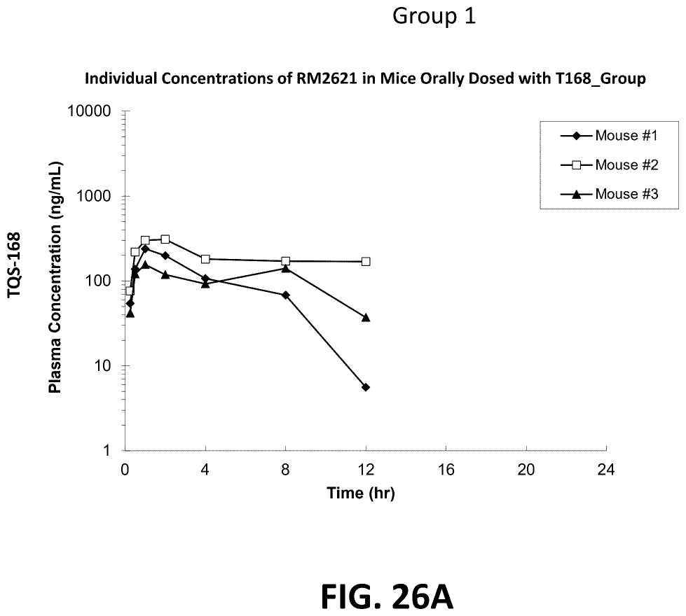

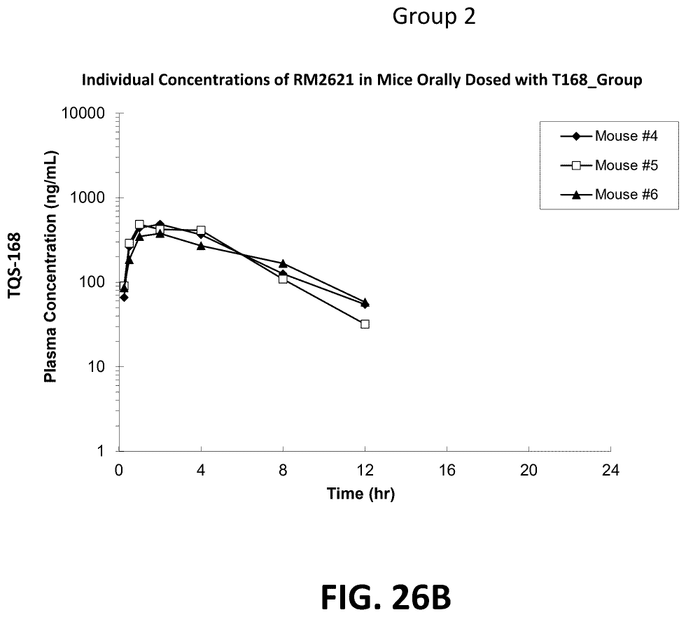

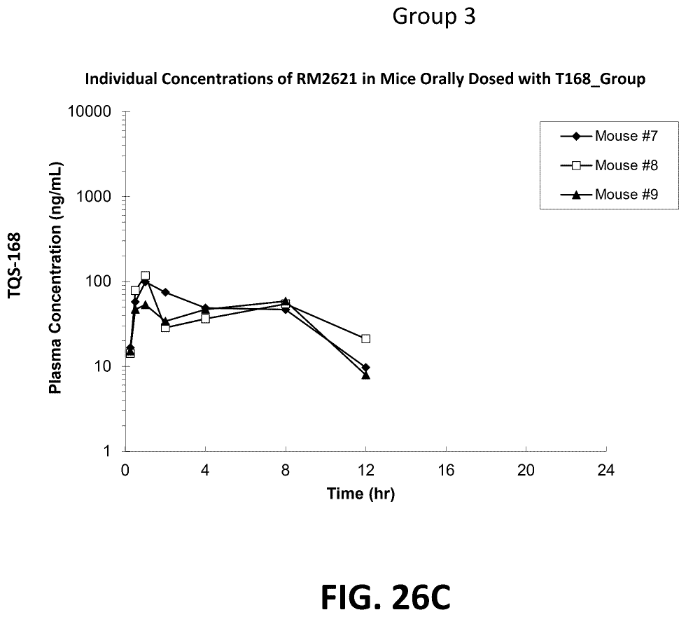

Results are plotted in FIG. 26A (Group 1), 26B (Group 2), and 26C (Group 3).

### 5.11. Example 11—TQS-621 Concentrations in Brain Following Single Oral Dose of Tqs-168 in Mice

TQS-168 was prepared as a 4.5 mg/ml solution in PEG400 10%| Kleptose 30% sterile water 60%. A single oral dose of 45 mg/kg was administered. Concentrations of TQS-621 were measured in plasma and brain over time. Data are provided in Tables 12 and 13 below.

TABLE 12
Plasma levels of TQS-621

| | | AUCₗₐₛₜ | AUC_{Inf} | Cₘₐₓ Ratio | AUCₗₐₛₜ Ratio |
|---|---|---|---|---|---|
| tₘₐₓ | Cₘₐₓ | (hr*ng/ | (hr*ng/ | (Metabolite/ | (Metabolites/ |
| (hr) | (ng/mL) | mL) | mL) | Parent) | Parent) |
| 4 | 692 | 4278 | 4311 | 0.299 | 0.315 |

TABLE 13
Brain levels of TQS-621

| | | | | | Cₘₐₓ | AUCₗₐₛₜ |
|---|---|---|---|---|---|---|
| | | | AUCₗₐₛₜ | AUC_{Inf} | Ratio | Ratio |
| t_{1/2} | tₘₐₓ | Cₘₐₓ | (hr*ng/ | (hr*ng/ | (Metabolite/ | (Metabolites/ |
| (hr) | (hr) | (ng/g) | g) | g) | Parent) | Parent) |
| 4.85 | 4 | 678 | 4234 | 4282 | 0.159 | 0.172 |

### 5.12. Example 12—TQS-168 Concentrations in Plasma and Brain Following Single Oral Dose in Rats

### Study Methodology

Both male (n=3) and female (n=3) Sprague-Dawley rats were administered a single dose of TQS-168 by oral gavage at 50, 150, or 500 mg/kg of TQS-168. TQS-168 was prepared as a suspension with 0.5% methylcellulose in PBS.

Plasma samples were collected at 30, 60, 120, 240, and 1440 minutes after administration. Brain samples were collected in some animals at 60, 240, and 1440 minutes after the single oral dose of TQS-168 at 500 mg/kg. Samples were processed for LC-MS analysis of TQS-168 concentrations.

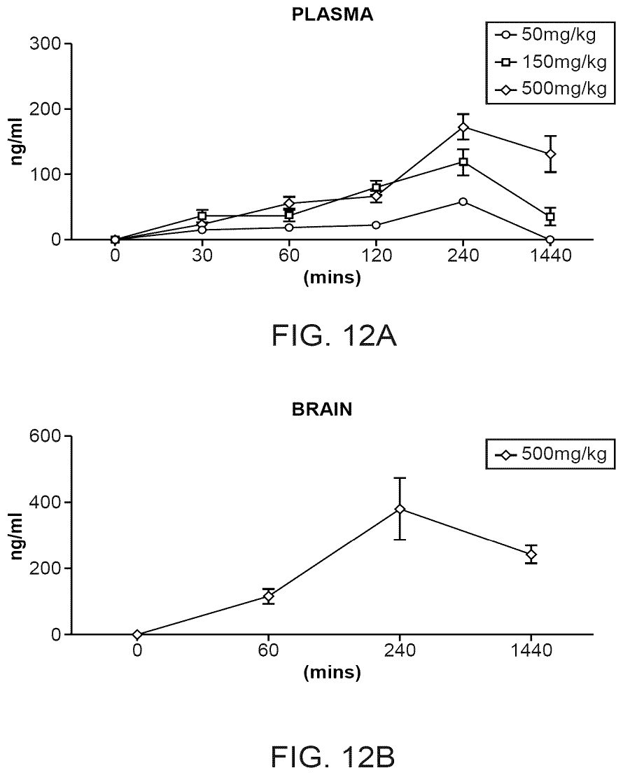

Data are presented as concentration of TQS-168 (ng/ml) in plasma (FIG. 12a) and brain homogenate (FIG. 12b). FIGS. 12a and 12b show that TQS-168 was detected in both plasma and brain tissues of treated rats. The maximum concentrations of TQS-168 (Cₘₐₓ) were detected at 240 minutes in both plasma and brain following oral administration of TQS-168.

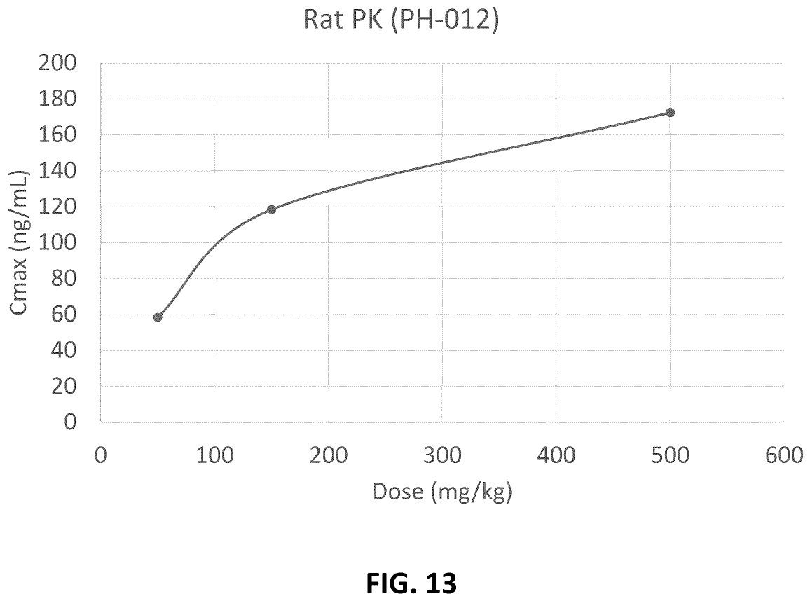

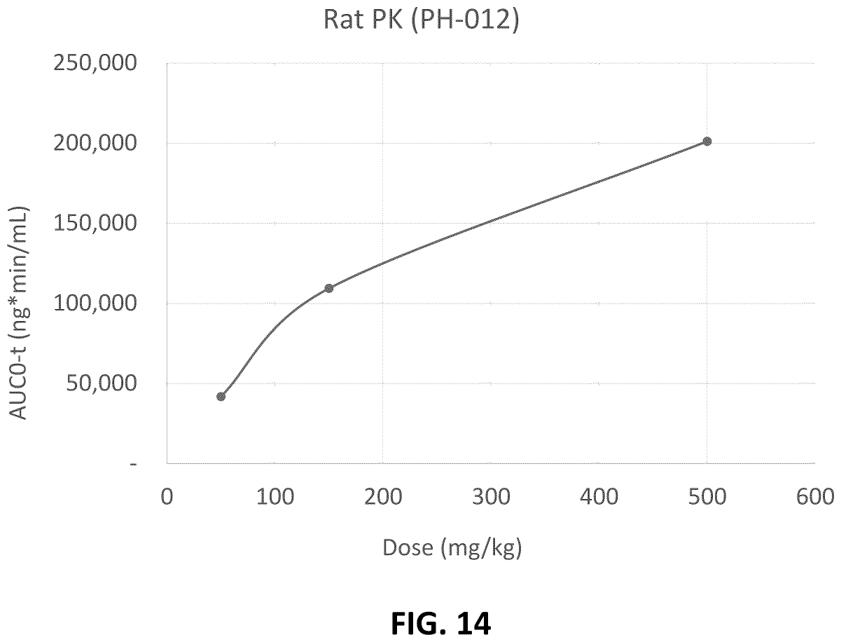

Cₘₐₓ and AUC₀₋ₜ values from the plasma tissues were calculated using PKSolver (Compute Methods Programs Biomed. 2010 September; 99(3):306-14. doi: 10.1016j.cmpb.2010.01.007. Epub 2010 Feb. 21) with the underlying data plotted FIG. 12a. The results are shown in FIG. 13 (Cₘₐₓ) and 14 (AUC), respectively, and the values are summarized in Table 14.

TABLE 14
Plasma Cₘₐₓ and AUC₀₋ₜ Values After Single Oral Dose in Rats

| | Cₘₐₓ | AUC₀₋ₜ | AUC₀₋ₜ |
|---|---|---|---|
| Dose | (ng/mL) | (hr*ng/mL) | (min*ng/mL) |
| 50 mg/kg | 60 | 833 | 50,000 |
| 150 mg/kg | 120 | 1,833 | 110,000 |
| 500 mg/kg | 170 | 3,333 | 200,000 |

### Conclusion

Following administration of a single oral dose at 50, 150, and 500 mg/kg in rats, TQS-168 was detected in both plasma and brain tissues with dose-dependent Cₘₐₓ and AUC.

### 5.13. Example 13—TQS-168 Plasma Concentrations Following Single Intravenous Dose in Mice

### Study Methodology

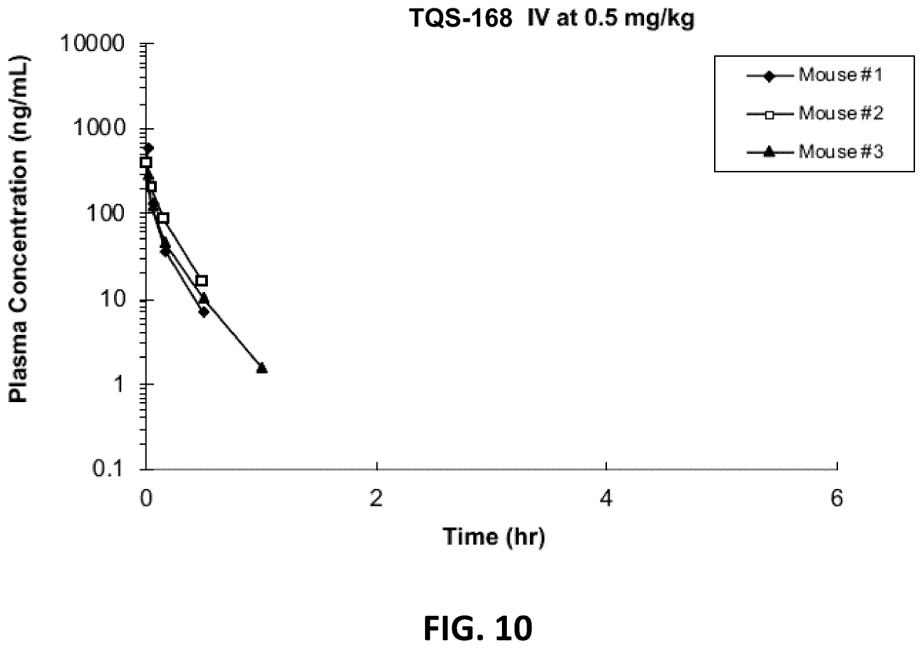

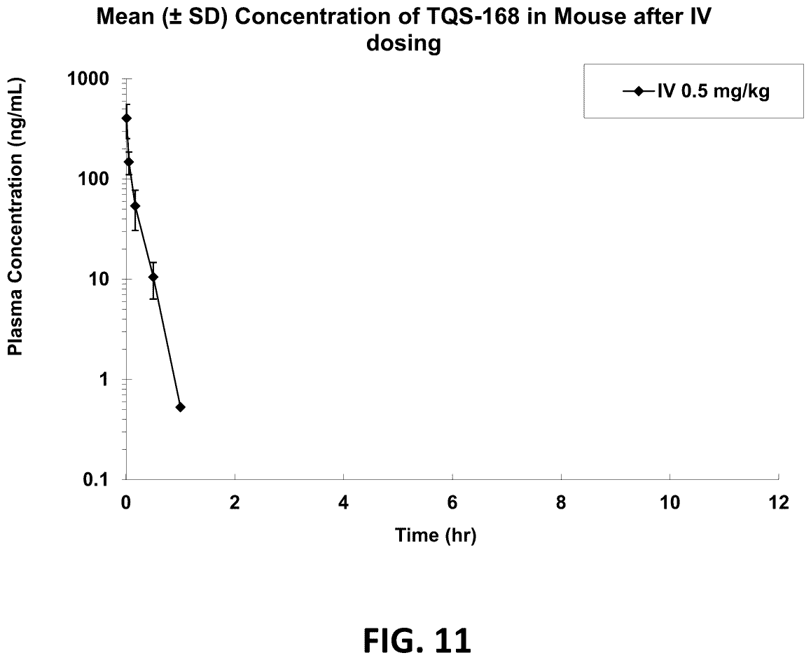

Three male 7-9 week old male CD-1 mice from Lingchang were treated intravenously with 0.5 mg/kg of TQS-168. TQS-168 was prepared in solution of 31.6% DMAC+36.8% Ethanol+31.6% Propylene glycol. Blood was collected at 0.5, 3, 10, 30, 60, 120, 240, 480 and 720 minutes after a single dose of TQS-168 and then processed. Blood samples were collected via saphenous vein puncture into a K₂EDTA tube, centrifuged at 4° C. at 4600 rpm for 5 minutes and plasma collected and stored at less than −20° C. prior to being analyzed by LC-MS for TQS-168 concentrations. Data are presented as concentration and mean concentration of TQS-168 per volume of plasma (ng/mL), as shown in FIG. 10 (individual mice) and FIG. 11 (average), respectively.

### Conclusion

Following intravenous dose of 0.5 mg/kg of TQS-168 in mice, TQS-168 was detected in the plasma. Terminal elimination half life was 0.14 hr (8.4 mins).

### 5.14. Example 14—Detection of TQS-168 in Plasma Following Single Intravenous Dose in Rats

### Study Methodology

A total of 3 male, 7-9 week old, Sprague Dawley rats from Vital River were administered a single intravenous dose of TQS-168 at 0.5 mg/kg, in a volume of 0.5 mL/kg. TQS-168 was prepared in solution of 31.6% DMAC+36.8% ethanol+31.6% propylene glycol.

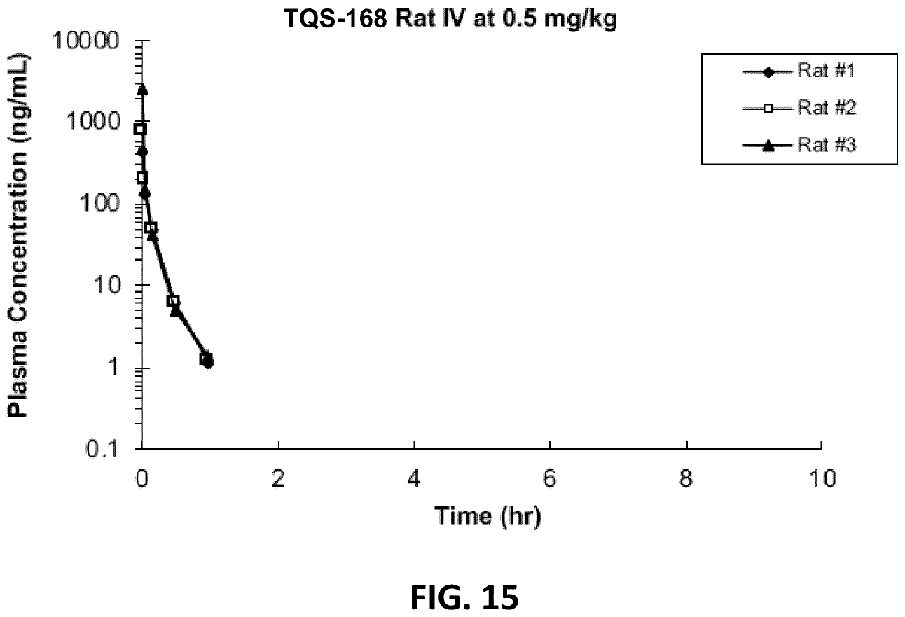

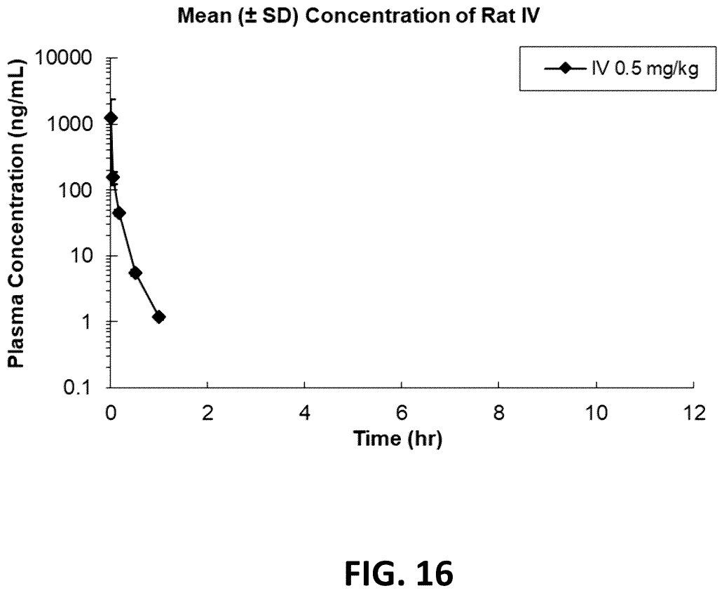

Blood samples were collected at 0.5, 3, 10, 30, 60, 120, 240, 480 and 720 minutes after a single dose of TQS-168 and then processed. Blood samples were collected via saphenous vein puncture into a K₂EDTA tube, centrifuged at 4° C. at 4600 rpm for 5 minutes, and plasma collected and stored at less than −20° C. prior to being analyzed by LC-MS for TQS-168 concentrations. Data are presented as concentrations of TQS-168 (ng/mL) in plasma and graphed in FIG. 15 (individual rats) and 16 (average).

### Conclusion

Following administration of a single intravenous dose of 0.5 mg/kg in rats, TQS-168 was detected in plasma. Half-life was 0.162 hrs (9.72 mins) with a clearance rate (L/hr/kg) of 9.29.

### 5.15. Example 15—Detection of TQS-168 in Plasma Following Single Intravenous Dose in Dogs

### Study Methodology

A total of 3 male 1-3 year old Beagle dogs from Beijing Marshall Biotechnology, Ltd were treated intravenously with 0.5 mg/kg of TQS-168 at 0.5 ml/kg. TQS-168 was prepared in solution containing 31.6% DMAC, 36.8% ethanol, and 31.6% propylene glycol.

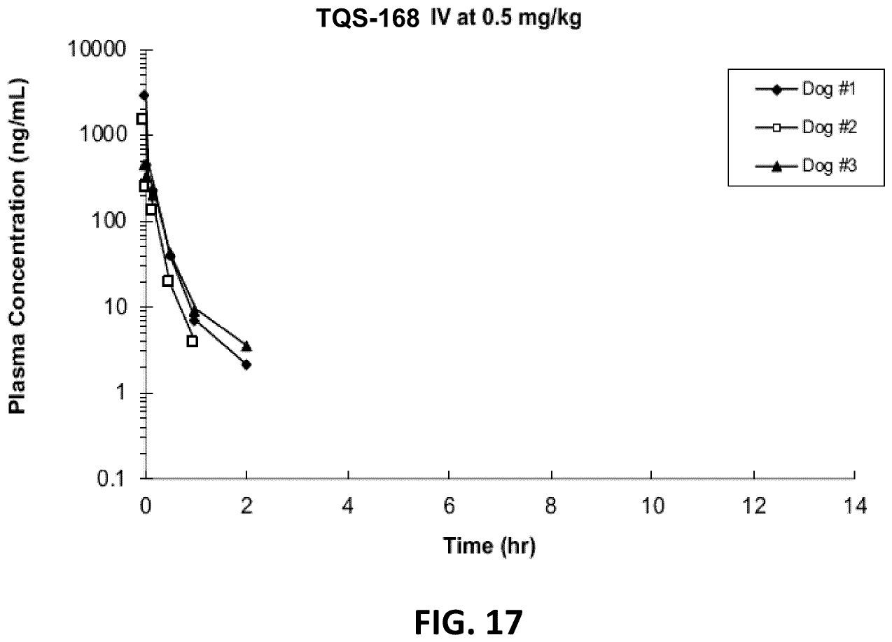

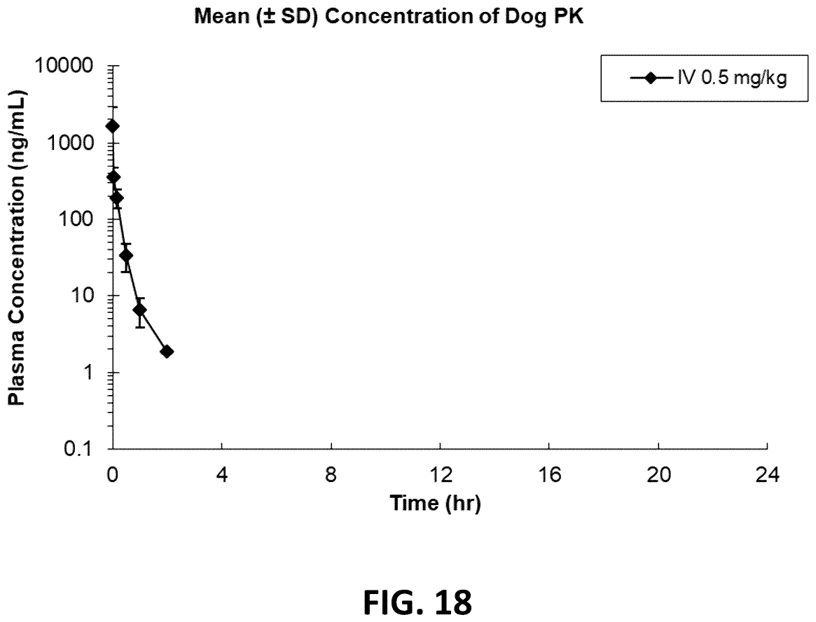

Blood samples were collected at 0.5, 3, 10, 30, 60, 120, 240, 360, 480, 720 and 1440 minutes after a single dose of TQS-168 and then processed. Blood samples were collected into a K₂EDTA tube, centrifuged at 4° C. at 4600 rpm for 5 minutes and plasma collected and stored at less than −20° C. prior to being analyzed by LC-MS for TQS-168 concentrations. Data are presented as concentration of TQS-168 per volume of plasma (ng/mL) and shown in FIGS. 17 and 18.

### 5.16. Example 16—Part 1: SAD Phase 1 Trial—Double-Blind Randomized Study of Subjects Receiving Single Ascending Doses of TQS-168 or Placebo

### 5.16.1. Trial Design

A double-blind, randomized, placebo-controlled clinical study was conducted to characterize and compare the pharmacokinetic (PK) profile of TQS-168 and its metabolite TQS-621 following single ascending doses of TQS-168, presented in 3 different formulations, or placebo, in healthy subjects.

Subjects: This randomized, double-blind, placebo-controlled phase 1 single ascending dose [SAD]trial was conducted in healthy male subjects aged 18 to 55 years with body mass index (BMI) 18.0 to 32.0 kg/m² as measured at screening. Subjects all weighed at least 55 kg at screening. Key criteria for exclusion were subjects with evidence of current SARS-CoV-2 infection, clinical manifestation of significant cardiovascular, renal, hepatic, dermatological, chronic respiratory or gastrointestinal disease, or aspartate aminotransferase (AST) or alanine aminotransferase (ALT)>1.5× the upper limit of normal (ULN). Subjects were recruited at a single site in the United Kingdom. Each subject provided written informed consent.

Trial Design: The trial was performed in multiple cohorts with a minimum of 7 subjects in each. In Cohorts 1-3 of Part 1, subjects received a single oral dose of TQS-168 methyl cellulose (MC) suspension, or spray dried dispersion (SDD) suspension, or hot melt extrusion (HME) suspension or placebo in the fasted state. Subjects were allocated to study treatment in a ratio of 6 TQS-168 to 2 placebo per cohort. Subjects in Cohort 1 were provided Regimen A, 60 mg TQS-168 MC. Subjects in Cohort 2 were provided Regimen B, 180 mg TQS-168. Cohort 3 was divided into three separate Periods, the subjects of which were the recipient of a single regimen. Subjects in Cohort 3 Period 1 were provided Regimen C, 540 mg TQS-168 MC suspension on day 1. The same subjects in Cohort 3 Period 2 were provided Regimen D, 180 mg TQS-168 SDD suspension on day 1, and in Part 1 Cohort 3 Period 2 the same subjects were provided Regimen E, 180 mg TQS-168 HME suspension on day 3. See Table 15.

TABLE 15
Part 1 Description of Regimens

| | Period | | | | | Route of |
|---|---|---|---|---|---|---|
| | (Dosing | | | | | Adminis- |
| Cohort | Day) | Regimen | IMP | Dose | | tration |
| 1 | N/A | A | TQS-168 MC | 60 | mg | Oral, |
| | | | Powder for Oral | | | Fasted |
| | | | Suspension | | | |
| | | | formulation or | | | |
| | | | placebo | | | |
| 2 | N/A | B | TQS-168 MC | 180 | mg | Oral, |
| | | | Powder for Oral | | | Fasted |
| | | | Suspension | | | |
| | | | formulation or | | | |
| | | | placebo | | | |
| 3 | 1 | C | TQS-168 MC | 540 | mg | Oral, |
| | | | Powder for Oral | | | Fasted |
| | | | Suspension | | | |
| | | | formulation or | | | |
| | | | placebo | | | |
| | 2 | D | TQS-168 SDD | 180 | mg | Oral, |
| | (Day 1) | | Powder for Oral | | | Fasted |
| | | | Suspension | | | |
| | | | formulation or | | | |
| | | | placebo | | | |
| | 2 | E | TQS-168 HME | 180 | mg | Oral, |

| | | |
|---|---|---|
| (Day 3) | Powder for Oral | Fasted |
| | Suspension | |
| | formulation or | |
| | placebo | |

IMP: investigational medicinal product;
N/A: not applicable.

For Cohorts 1-3 Period 1, the screening period was up to 4 weeks. After confirming eligibility, subjects within each cohort were randomly assigned to receive either the active (TQS-168) or placebo treatment. Note, this is the first time TQS-168 has been dosed in humans, and therefore a sentinel dosing design was followed. Each cohort was split into a sentinel group and a main group. The sentinel group consisted of the first two subjects of each cohort. They were dosed prior to the remaining subjects, the main group. Only after a positive review of the safety data of the sentinel group up to 24 h post-dose were the main group subjects in the cohort dosed. The randomization schedule was constructed such that one of the subjects dosed on the first day received the TQS-168 suspension and one received the placebo. Per protocol, all treatments were to be taken once daily following an overnight fast (≥10 hours fasted) except in cohorts in which the effect of food was evaluated. For Cohorts 1-3 Period 1, subjects were admitted in the morning on the day before dosing (Day −1) and remained onsite until their discharge, 48 hours post-dose (Day 3). For Cohorts 3 Period 2, subjects were admitted in the morning on the day before dosing (Day −1) and remained onsite until their discharge, 48 hours post-dose (Day 5). Per protocol, subjects were dosed on the morning of Day 1 either in the fasted state, following an overnight fast (≥10 hours) or fed state, following a high-fat breakfast given 30 minutes before dosing, depending on their assigned regimen.

Post-dosing of Cohorts 1-3 Period 1, and prior to dosing subjects in Cohort 3 Period 2, an interim period was observed, and a safety review was performed. The review concluded that it was safe to dose subjects with the SDD and HME formulations at similar dosage as the MC formulation. As a result, sentinels were not required for Cohort 3 Period 2.

In cohorts 4-6, each subject received a single oral dose of TQS-168 SDD suspension or placebo in the fed or fasted state. Subjects were randomly allocated to study treatment versus placebo per cohort in a 5:1 ratio. Cohort 4 received Regimen F, 90 mg TQS-168 administered to subject in the fed state. Cohort 5 received Regimen G, 90 mg TQS-168 administered to subject in the fasted state. Cohort 6 received Regimen H, 270 mg TQS-168 administered to subject in the fasted. Per protocol, subjects were dosed on the morning of Day 1 either in the fasted state following an overnight fast (≥10 hours) or fed state following a high-fat breakfast given 30 minutes before dosing. See Table 16.

Safety was continually assessed throughout the trial by monitoring adverse events and concomitant medication use, electrocardiograms (ECGs), vital signs, laboratory safety assessments and physical examinations. Blood samples for pharmacokinetic assessments were collected from each subject from Day −1, prior (≤1 hr) to each dose, and at intervals throughout the study until 48 hours post-dose as applicable. In Cohorts 1-5, no dose was used that was expected to exceed the pre-trial designated exposure cap of Cₘₐₓ 305 ng/mL and AUC₍₀₋₂₄₎ of 2750 ng*h/mL, in any individual subject. After the protocol amendment, this exposure cap was increased to Cmax 1121 ng/mL and AUC 5969 ng*h/mL.

TABLE 16
Part 1 Description of Regimens

| | Period | | | | | Route of |
|---|---|---|---|---|---|---|
| | (Dosing | | | | | Adminis- |
| Cohort | Day) | Regimen | IMP | Dose | | tration |
| 4 | N/A | F | TQS-168 SDD | 90 | mg | Oral, |
| | | | Powder for Oral | | | Fed |
| | | | Suspension | | | |
| | | | formulation or | | | |
| | | | placebo | | | |
| 5 | N/A | G | TQS-168 SDD | 90 | mg | Oral, |
| | | | Powder for Oral | | | Fasted |
| | | | Suspension | | | |
| | | | formulation or | | | |
| | | | placebo | | | |
| 6 | N/A | H | TQS-168 SDD | 270 | mg | Oral, |

| | |
|---|---|
| Powder for Oral | Fasted |
| Suspension | |
| formulation or | |
| placebo | |

IMP: investigational medicinal product;
N/A: not applicable.

For all subjects in Cohorts 1-3, safety was continually assessed throughout the trial by monitoring adverse events and concomitant medication use, electrocardiograms (ECGs), vital signs, laboratory safety assessments and physical examinations. Blood samples for pharmacokinetic assessments were collected from each subject prior from Day −1, (≤1 hr) to each dose, and at intervals throughout the study until 48 hours post-dose as applicable.

Assessment of Pharmacokinetics: Blood samples for plasma PK analysis were collected at regular time intervals. Venous blood samples were collected from the subjects by a trained member of the clinical team. Pre-dose samples were taken ≤1 h before dosing. Timestamp 0 to 1 hour post-dose samples were taken within ±2 minutes of the nominal post-dose sampling time. Timestamp 1.5 to 12 hour post-dose samples were taken within ±10 min of the nominal post-dose sampling time. Timestamp 16 to 48 hour post-dose samples will be taken within +30 minutes of the nominal post-dose sampling time. Samples were collected into appropriate containers and were processed to isolate plasma. PK analysis were carried out on plasma samples using validated bioanalytical methods.

Statistical Analyses: The sample sizes for the study were chosen based on practical considerations and experience from previous studies of a similar design. The numbers of subjects in each cohort (group) were considered to be adequate to assess the main objectives of each study. Pharmacokinetic parameters were determined by non-compartmental techniques using WinNonlin software version 8.0 or higher (Certara USA. Inc., USA). All data were listed and summarized by subject group using descriptive statistics. All statistical analyses were conducted using SAS version 9.4 or higher.

### 5.16.2. TQS-168 Methylcellulose (MC) Powder Suspension Formulation

A methylcellulose (MC) powder suspension formulation of 2-(4-tert-butylphenyl)-1H-benzimidazole (TQS-168; compound of formula I) was prepared by reconstitution as a suspension in a methylcellulose vehicle formulation of Table 17 (“the vehicle formulation”).

TABLE 17
Vehicle formulation for preparation of MC powder
suspension formulation of compound of formula I

| Component | % w/w | Amount (g) |
|---|---|---|
| methylcellulose (Methocel A4C | 0.5 | 10.0 |
| Premium, supplied by Dupont, | | |
| yields viscosity of 400 cP) | | |
| sodium dodecyl sulfate (Kolliphor | 0.10 | 2.0 |
| SLS Fine, BASF 151-21-3) | | |
| 30% simethicone emulsion | 0.10 | 2.0 |
| (supplied by Dow-Corning, Q7-2587 | | |
| oil in water (O/W) emulsion | | |
| with 30.4% polydimethylsiloxane | | |
| (PDMS), 1.2-2.1% SiO₂ particles) | | |
| water | q.s. to | q.s. to |
| | 100 | 2000.0 g |
| Total | 100 | 2000 |

The Vehicle formulation was prepared by heating the water (1986 g) to 80° C. (+5° C.) then adding the methylcellulose (10 g) and stirring for 30 minutes or more until the methyl cellulose was fully dispersed. Sodium dodecyl sulfate (2 g) and 30% simethicone emulsion (2 g) were then added, and the mixture was stirred until a translucent, white/off-white, slightly viscous suspension, free form particulate was formed. The pH of the resulting vehicle formulation was 5.3 (target pH was 6.0+/−3.0).

### General Procedure for Reconstitution of Compound of Formula I in the Vehicle Formulation of Table 17

The required amount of 2-(4-tert-butylphenyl)-1H-benzimidazole (compound 1; TQS-168) (e.g., from 60 mg-1000 mg) was weighed into a vial. The vehicle formulation (100 mL) was added to the vial containing compound of formula I to obtain the compound of formula I methylcellulose (MC) powder suspension formulation.

### 5.16.3. Methylcellulose (MC) Results

Healthy male subjects were administered a single oral dose of TQS-168 methylcellulose (MC) powder at one of the following doses in the fasted state: 60 mg, 180 mg or 540 mg. Plasma concentrations of TQS-168 and metabolite TQS-621 were measured over time, and key pharmacokinetic parameters determined.

Table 18 and Table 19 present the geometric mean of TQS-168 and metabolite TQS-621 key pharmacokinetic parameters in the subjects following oral administration of TQS-168.

TABLE 18
Geometric Mean (CV %) key pharmacokinetic parameters methylcellulose formulation

| Treatment | | Tmax* | Cmax | AUC(0-24) | AUC(0-last) | AUC(0-inf) | T_{1/2} |
|---|---|---|---|---|---|---|---|
| Part 1 | Analyte | (h) | (ng/mL) | (ng · h/mL) | (ng · h/mL) | (ng · h/mL) | (h) |
| Cohort 1 | TQS-168 | 1.00 | 26.7 | 71.2 | 67.6 | 72.7 | 3.06 |
| Regimen A | | (0.5-2.0) | (46.6) | (52.2) | (54.2) | (53.4) | (61.1) |
| 60 mg TQS-168 | TQS-621 | 2.50 | 65.3 | 383 | 401 | 410 | 4.89 |
| MC Powder for | | (1.0-4.0) | (37.5) | (76.4) | (81.7) | (81.4) | (69.7) |
| Oral Suspension | | | | | | | |
| Cohort 2 | TQS-168 | 1.00 | 53.1 | 163 | 168 | 180 | 7.36 |
| Regimen B | | (1.00-4.00) | (55.0) | (46.3) | (51.8) | (53.1) | (75.5) |
| 180 mg TQS-168 | TQS-621 | 1.50 | 199 | 1210 | 1320 | 1350 | 9.10 |
| MC Powder for | | (1.00-4.00) | (33.5) | (42.2) | (45.9) | (45.1) | (85.2) |
| Oral Suspension | | | | | | | |
| Cohort 3 Period 1 | TQS-168 | 2.25 | 323 | 1440 | 1540 | 1600 | 10.1 |
| Regimen C | | (1.00-10.0) | (70.0) | (74.5) | (71.0) | (67.6) | (81.8) |
| 540 mg TQS-168 | TQS-621 | 4.00 | 1110 | 10800 | 12000 | 12700 | 11.3 |
| MC Powder for | | (2.00-12.00) | (42.5) | (68.2) | (68.9) | (64.9) | (73.0) |
| Oral Suspension | | | | | | | |

*Median (min-max)

TABLE 19
Geometric Mean (CV %) key pharmacokinetic
parameters methylcellulose formulation

| Treatment | | Cmax | MPR | AUC(0-inf) | MPR |
|---|---|---|---|---|---|
| Part 1 | Analyte | (ng/mL) | Cmax | (ng · h/mL) | AUC(0-inf) |
| Cohort 1 | TQS-168 | 26.7 | — | 72.7 | — |
| (Regimen A) | | (46.6) | | (53.4) | |
| 60 mg TQS-168 | TQS-621 | 65.3 | 2.44 | 410 | 5.63 |
| MC Powder for | | (37.5) | (53.0) | (81.4) | (38.5) |
| Oral Suspension | | | | | |
| Cohort 2 | TQS-168 | 53.1 | — | 180 | — |
| (Regimen B) | | (55.0) | | (53.1) | |
| 180 mg TQS-168 | TQS-621 | 199 | 3.74 | 1350 | 7.52 |
| MC Powder for | | (33.5) | (65.1) | (45.1) | (29.4) |
| Oral Suspension | | | | | |
| Cohort 3 Period 1 | TQS-168 | 323 | — | 1600 | — |
| (Regimen C) | | (70.0) | | (67.6) | |
| 540 mg TQS-168 | TQS-621 | 1110 | 3.43 | 12700 | 7.91 |
| MC Powder for | | (42.5) | (40.3) | (64.9) | (19.1) |
| Oral Suspension | | | | | |

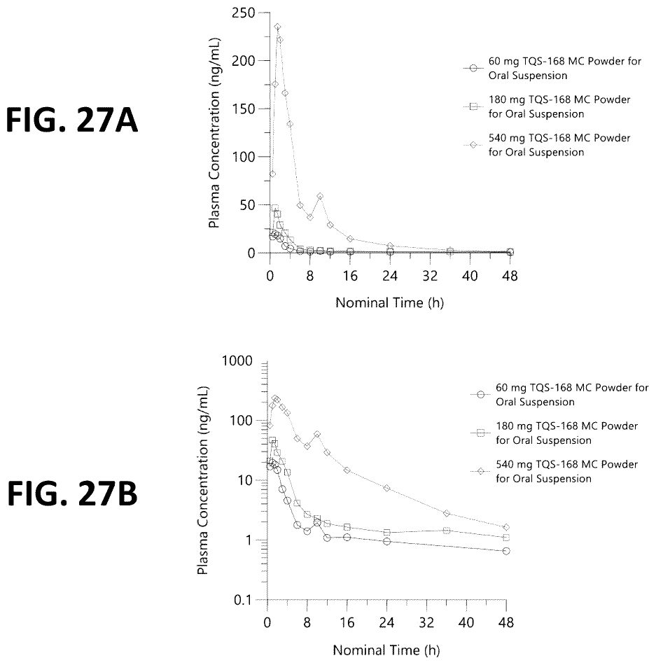

TQS-168 single ascending dose (SAD) PK profile: Cohort 3 Period 1 received 540 mg TQS-168 (Regimen C) and provided the highest TQS-168 Cₘₐₓ of 323 ng/mL (1.29 μM). Cohort 2 received 180 mg TQS-168 (Regimen B) and provided a TQS-168 Cₘₐₓ of 53.1 ng/mL (0.21 μM). Cohort 1 received 60 mg TQS-168 (Regimen A) and provided a TQS-168 Cₘₐₓ of 26.7 ng/mL (0.11 μM). Results are plotted in FIG. 27. Cohort 3 Period 1 also displayed higher exposure (AUC₀₋₂₄=1440 hr*ng/mL, AUC_{0-inf}=1600 hr*ng/mL) than Cohort 2 (AUC₀₋₂₄=163 hr*ng/m, AUC_{0-inf}=180 hr*ng/mL) and Cohort 1 (AUC₀₋₂₄=71.2 hr*ng/mL, AUC_{0-inf}=72.7 hr*ng/mL).

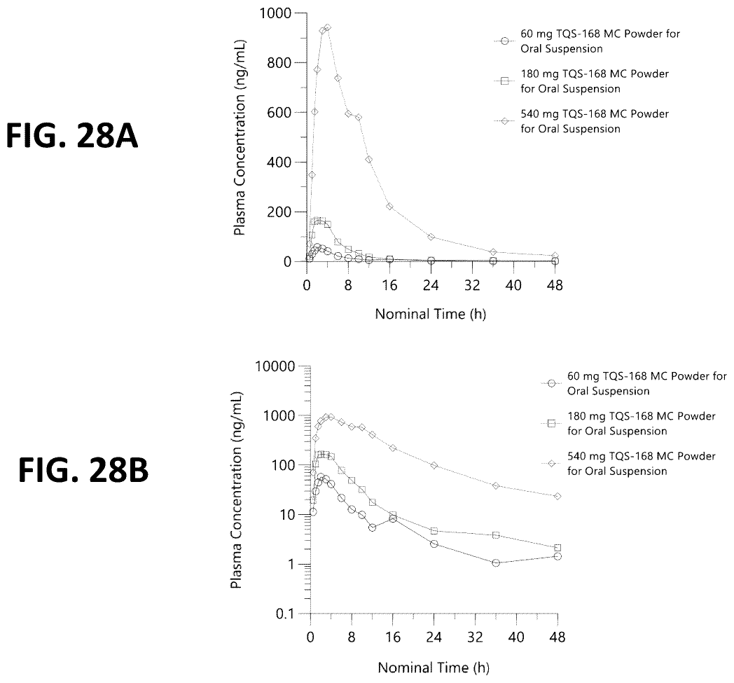

Metabolite TQS-621 PK profile: Cohort 3 Period 1 displayed higher metabolite TQS-621 Cₘₐₓ (1110 ng/mL) (4.17 μM) than Cohort 2 (199 ng/mL) (0.75 μM) and Cohort 1 (65.3 ng/mL) (0.25 μM). Results are plotted in FIG. 28. Similarly, Cohort 3 also displayed higher metabolite exposure (AUC₀₋₂₄=10800 hr*ng/m, AUC_{inf}=12700 hr*ng/m) than Cohort 2 (AUC₀₋₂₄=1210 hr*ng/mL, AUC_{inf}=1350 hr*ng/mL) and Cohort 1 (AUC₀₋₂₄=383 hr*ng/mL, AUC_{inf}=410 hr*ng/mL).

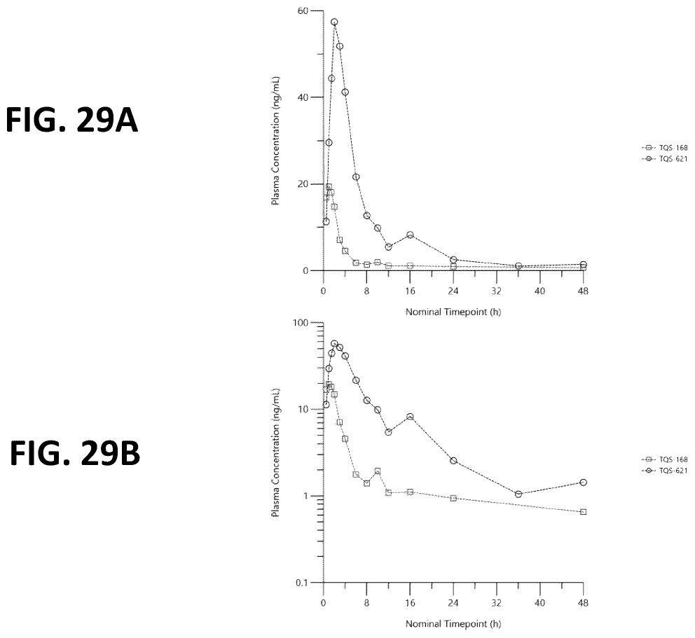

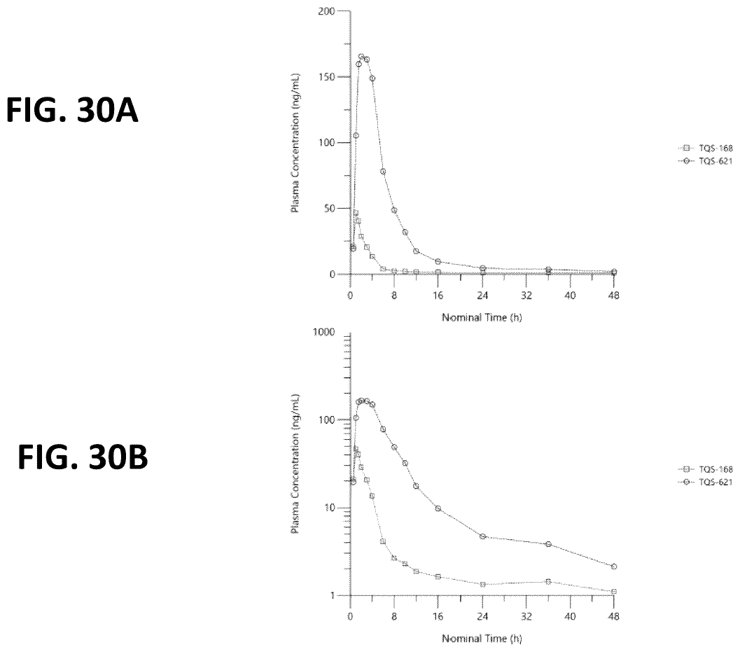

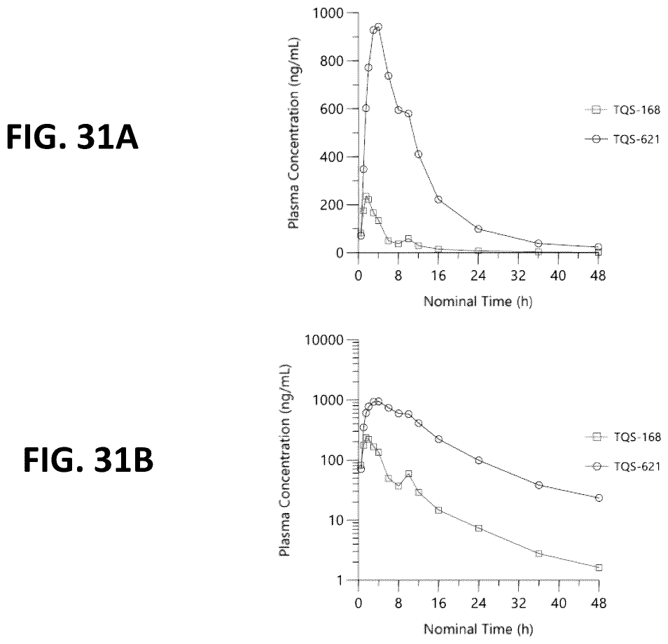

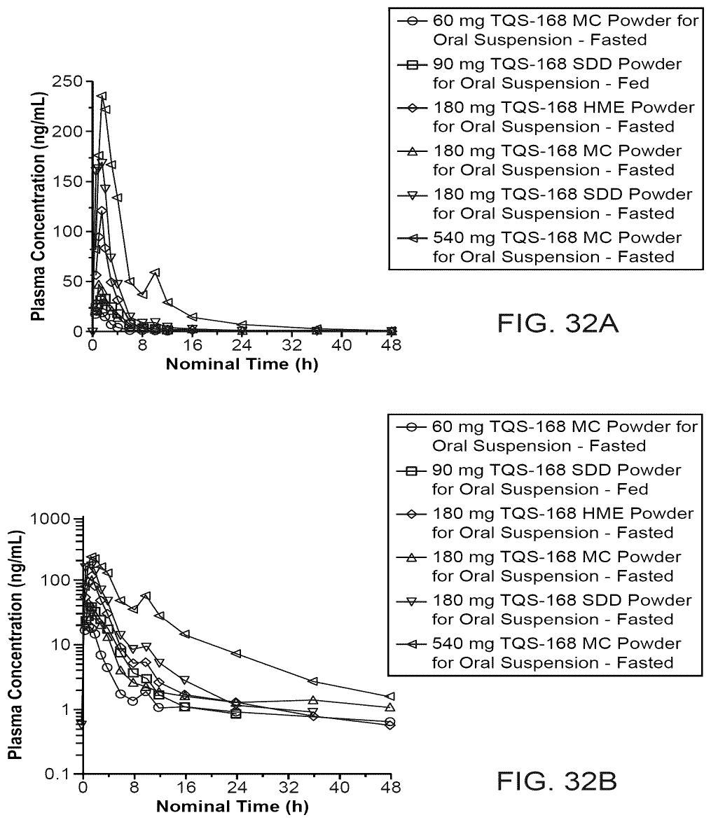

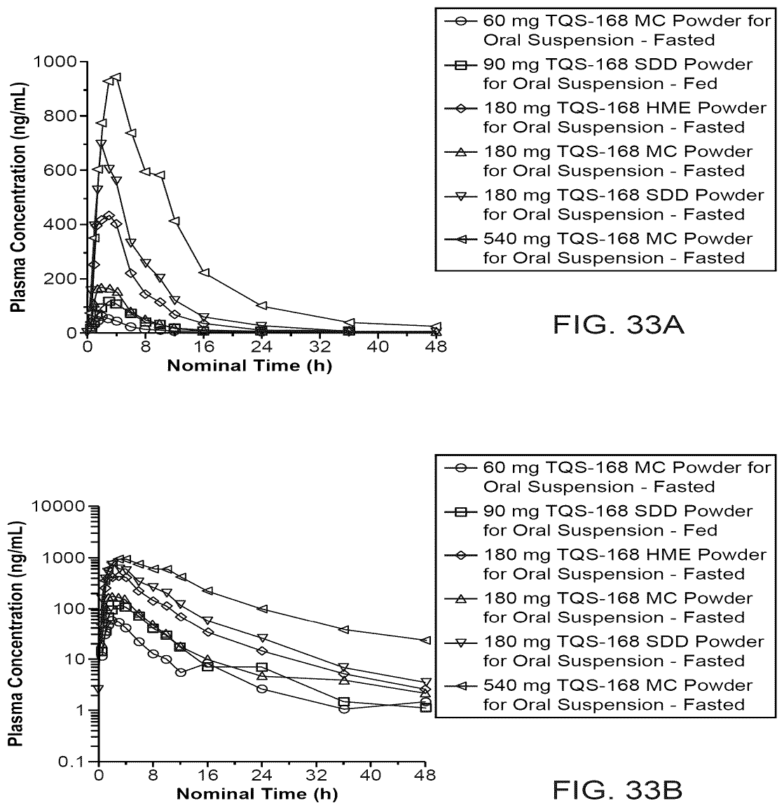

In the study, maximal plasma concentrations for TQS-168 (Cₘₐₓ), and TQS-168 exposures (AUC₍₀₋₂₄₎ and AUC_{(0-inf)}) appeared to increase slightly sub-proportionally to dose following single doses of 60 and 180 mg TQS-168. Note, however, that between doses 180 mg and 540 mg of TQS-168, Cₘₐₓ, AUC₍₀₋₂₄₎ and AUC_{(0-inf)} for TQS-168 increased in a supra-proportional manner, with a 6.1-, 8.8- and 9.5-fold increase, respectively for a 3-fold increase in dose. When observing the entire dose range from 60 mg-540 mg, Cₘₐₓ, AUC₍₀₋₂₄₎ and AUC_{(0-inf)} all increased supra-proportionally by 12.1-fold, 20.2-fold and 21.2-fold respectively. See FIGS. 27, 29-31.

Regarding metabolite TQS-621, plasma Cₘₐₓ, AUC₍₀₋₂₄₎ and AUC_{(0-inf)} appeared to increase proportionally to dose following single doses from 60 to 180 mg TQS-168. For doses between 180 and 540 mg, Cₘₐₓ, AUC₍₀₋₂₄₎ and AUC_{(0-inf)} increased supra-proportionally with a 5.6-, 8.9- and 9.4-fold increase, respectively for a 3-fold increase in dose. Furthermore, over the entire dose range, a 9-fold increase in dose from 60 mg to 540 mg, plasma Cₘₐₓ, AUC₍₀₋₂₄₎ and AUC_{(0-inf)} increased supra proportionally by 17.0-, 28.2- and 31.0-fold respectively. See FIGS. 28-31.

Following a dose of 60 mg, maximum plasma TQS-168 concentrations occurred between 0.5 and 2 hours post-dose, with a median Tₘₐₓ of 1 hour post-dose for TQS-168. Maximum plasma metabolite TQS-621 concentrations occurred between 1-4 hours post dose, with a median Tₘₐₓ of 2.5 hours post-dose.

Following a dose of 180 mg TQS-168, the maximum plasma concentrations of TQS-168 occurred between 1 and 4 hours post-dose. A median Tₘₐₓ of 1 hour post-dose was observed. Maximum plasma metabolite TQS-621 concentrations occurred between 1-4 hours post dose, with a median Tₘₐₓ of 1.5 hours post-dose.

Following administration of 540 mg TQS-168, the maximum plasma concentration of TQS-168 occurred between 1 and 10 hours post-dose, with a median Tₘₐₓ of 2.25 hours post-dose. Metabolite TQS-621 displayed maximum plasma concentrations between 2 and 12 hours post dose, with a median Tₘₐₓ of 4 hours post-dose.

At all administered doses of TQS-168, the concentrations of metabolite TQS-621 exceeded concentrations of TQS-168 following a short lag.

The geometric mean terminal half-life of TQS-168 following the TQS-168 60 mg dose, 180 mg dose and 540 mg dose was dose-dependent at 3.06, 7.36 and 10.1 hours, respectively. The geometric mean terminal half-life of metabolite TQS-621 for TQS-168 doses 60 mg, 180 mg and 540 mg was 4.89, 9.10 and 11.3 hours, respectively. It is noted that for all doses of TQS-168, plasma concentrations of TQS-168 were quantifiable from 0.5 hours post-dose and remained quantifiable up to the final sample time point of 48 hours post-dose. Concentrations of TQS-621 were also quantifiable from 0.5 hours post-dose and remained quantifiable up to the final sampling time point of 48 hours post-dose.

### 5.16.4. Spray-Dried Dispersion (SDD) Formulation and Oral Suspension of the Same

### 5.16.4.1 Preparation of a SDD Formulation of 2-(4-tert-butylphenyl)-1H-benzimidazole (Compound of Formula I)

A spray-dried dispersion (SDD) of 2-(4-tert-butylphenyl)-1H-benzimidazole (compound of formula I; TQS-168) having the composition set out in Table 20 was prepared by spray drying a feedstock formulation set out in Table 21.

TABLE 20
Spray dried dispersion (SDD) formulation
of compound of formula i

| | | Amount |
|---|---|---|
| Ingredient | % w/w | (g) |
| Compound of formula I (TQS-168) | 30 | 45.0 |
| polyvinyl caprolactam-polyvinyl acetate-polyethylene | 40 | 60.0 |
| glycol graft co-polymer (Soluplus) | | |
| amorphous silicon dioxide (Syloid ® 244 FP) | 30 | 45.0 |
| Total | 100 | 150 |

TABLE 21
Spray drying feedstock formulation

| | | Amount |
|---|---|---|
| Ingredient | % w/w | (g) |
| Compound of formula I | 2.3 | 45.0 |
| polyvinyl caprolactam-polyvinyl acetate-polyethylene | 3.1 | 60.0 |
| glycol graft co-polymer (Soluplus) | | |
| amorphous silicon dioxide (Syloid ® 244 FP) | 2.3 | 45.0 |
| 2-propanol | 92.3 | 1797.1 |
| Total | 100 | 1947.1 |

### 5.16.4.2 Manufacturing Procedure for Spray Drying Feedstock Formulation

Compound of formula I (45.0 g) was slowly added to 2-propanol (1791.1 g) with stirring, placed under a homogenizer (Silverson SL2 homogenizer) and stirred for 5 minutes or more until Compound of formula I was fully dissolved. The reaction mixture was then removed from the homogenizer and polyvinyl caprolactam-polyvinyl acetate-polyethylene glycol graft co-polymer (Soluplus) (60.0 g) was slowly added with stirring, placed back under the homogenizer and stirred for 10 minutes or more until the Soluplus was fully dissolved. The reaction mixture was then removed from the homogenizer and amorphous silicon dioxide (Syloid® 244 FP) was slowly added with stirring, placed back under the homogenizer and stirred for an additional 15 minutes or more until the amorphous silicon dioxide was fully dispersed. The resulting suspension is referred to herein as the “Feedstock Formulation.”

### 5.16.4.3 Manufacturing Procedure for Spray Dried Dispersion (SDD) Formulation of Compound of Formula I

The spray dryer unit (ProCepT 4M8 Spray Dryer) was set up with a compressed air supply. Once the outlet temperature stabilized, the feed pump was initiated and 2-propanol (blank solution) was sprayed through the nozzle as a fine spray into the collection chamber. The spray dryer parameters were adjusted to achieve a feed rate within the range set out in Table 22 below.

The feedstock formulation was stirred under a homogenizer at a speed appropriate to maintain a homogenous dispersion without generating bubbles. The feedstock formulation was then sprayed through the nozzle as a fine spray into the collection chamber of the spray dryer unit (ProCepT 4M8 Spray Dryer, using parameters as set up with the blank solution and outlined in Table 3) where the solvent was evaporated quickly to generate particles containing compound of formula I polyvinyl caprolactam-polyvinyl acetate-polyethylene glycol graft co-polymer (Soluplus) and silicon dioxide (Syloid® 244 FP) (SDD formulation of compound 1). Once all the feedstock formulation had been sprayed and collected, the feedstock formulation was replaced with 2-propanol (blank solution) and sprayed through the nozzle of the spray dryer for 5 minutes or more to allow collection of any remaining “feedstock formulation” within the air stream.

TABLE 22
Target Process Parameters for Spray Dryer

| Process Parameter | Target | |
|---|---|---|
| Inlet air flow | 0.45 | m³/min |
| Inlet air temperature | 88° | C. |
| Suspension feed rate | 4-7 | g/min |
| Peristaltic tubing | 50513 Tygon MHLL ID 2.79 mm | |
| | (2-stop, purple-white) | |
| Nozzle diameter | 0.6 | mm |
| Nozzle cap | with cap | |
| Nozzle pressure | 2 | bar |
| Cyclone gas pressure | 0.3 | bar |

### 5.16.4.4 Oral Suspension of a Spray Dried Dispersion (SDD) Formulation of 2-(4-tert-butylphenyl)-1H-benzimidazole (Compound 1)

The spray-dried dispersion (SDD) of 2-(4-tert-butylphenyl)-1H-benzimidazole (compound 1) having the composition set out in Table 20 (e.g., from 60-1000 mg) was reconstituted as an oral suspension in 100 g of vehicle composed of PEG 300 (10 g), glycerol monocaprylocaprate (Capmul MCM, 0.40 mg) in sterile water for irrigation (q.s. to 100 g).

### 5.16.5. SDD Results

Healthy male subjects were administered a single oral dose of TQS-168 SDD powder oral suspension at 90 mg or 180 mg or 270 mg in the fed or fasted state. Plasma concentrations of TQS-168 and metabolite TQS-621 were measured over time and key pharmacokinetic parameters determined.

Table 23 and Table 24 present TQS-168 and metabolite TQS-621 key pharmacokinetic parameters in the subjects following oral administration of TQS-168 SDD formulation.

TABLE 23
Geometric Mean (CV %) key pharmacokinetic parameters SDD formulation

| Treatment | | Tmax* | Cmax | AUC(0-24) | AUC(0-last) | AUC(0-inf) | T_{1/2} |
|---|---|---|---|---|---|---|---|
| Part 1 | Analyte | (h) | (ng/mL) | (ng · h/mL) | (ng · h/mL) | (ng · h/mL) | (h) |
| Cohort 3 Part2 | TQS-168 | 1.00 | 218 | 608 | 611 | 621 | 5.64 |
| Regimen D | | (0.50-2.00) | (77.2) | (78.5) | (78.8) | (77.4) | (32.4) |
| 180 mg TQS-168 | TQS-621 | 2.00 | 742 | 4860 | 5110 | 5170 | 7.12 |
| SDD Powder for Oral | | (1.50-3.00) | (39.9) | (37.3) | (35.8) | (35.2) | (32.4) |
| Suspension in the fasted | | | | | | | |
| state | | | | | | | |
| Cohort 4 | TQS-168 | 1.50 | 47.1 | 168 | 166 | 171 | 3.52 |
| Regimen F | | (0.50-3.00) | (39.1) | (53.5) | (54.2) | (53.3) | (35.8) |
| 90 mg TQS-168 | TQS-621 | 3.50 | 122 | 848 | 894 | 901 | 5.17 |
| SDD Powder for Oral | | (1.50-6.00) | (36.1) | (57.6) | (61.6) | (61.5) | (47.5) |
| Suspension in the Fed State | | | | | | | |
| (high fat breakfast) | | | | | | | |
| Cohort 5 | TQS-168 | 0.50 | 111 | 192 | 189 | 195 | 4.85 |
| Regimen G | | (0.50-4.00) | (92.2) | (58.5) | (60.0) | (58.3) | (45.1) |
| 90 mg TQS-168 | TQS-621 | 1.25 | 214 | 1170 | 1190 | 1200 | 4.79 |
| SDD Powder for Oral | | (1.00-3.00) | (44.7) | (50.4) | (52.5) | (52.4) | (58.3) |
| Suspension in the Fasted | | | | | | | |
| State | | | | | | | |
| Cohort 6 | TQS-168 | 1.50 | 237 | 736 | 801 | 836 | 10.4 |
| Regimen H | | (0.500-4.00) | (245.4) | (170.3) | (151.2) | (137.3) | (41.6) |
| 270 mg TQS-168 | TQS-621 | 2.00 | 621 | 4250 | 4760 | 4950 | 10.4 |
| SDD Powder for Oral | | (1.50-4.00) | (96.0) | (106.4) | (101.0) | (97.1) | (21.9) |
| Suspension in the Fasted | | | | | | | |
| State | | | | | | | |

*Median (min-max)

TABLE 24
Geometric Mean (CV %) key pharmacokinetic parameters SDD formulation

| Treatment | | Cmax | MPR | AUC(0-inf) | MPR |
|---|---|---|---|---|---|
| Part 1 | Analyte | (ng/mL) | Cmax | (ng · h/mL) | AUC(0-inf) |
| Cohort 3 Part2 Regimen D | TQS-168 | 218 | — | 621 | — |
| 180 mg TQS-168 SDD | | (77.2) | | (77.4) | |
| Powder for Oral Suspension | TQS-621 | 742 | 3.41 | 5170 | 8.32 |
| in the fasted state | | (39.9) | (52.9) | (35.2) | (42.8) |
| Cohort 4 Regimen F 90 mg | TQS-168 | 47.1 | — | 171 | — |
| TQS-168 SDD Powder for | | (39.1) | | (53.3) | |
| Oral Suspension in the Fed | TQS-621 | 122 | 2.60 | 901 | 5.26 |
| State (high fat breakfast) | | (36.1) | (42.4) | (61.5) | (34.4) |
| Cohort 5 Regimen G 90 mg | TQS-168 | 111 | — | 195 | — |
| TQS-168 SDD Powder | | (92.2) | | (58.3) | |
| for Oral Suspension | TQS-621 | 214 | 1.93 | 1200 | 6.16 |
| in the Fasted State | | (44.7) | (52.0) | (52.4) | (10.5) |
| Cohort 6 Regimen H | TQS-168 | 237 | — | 836 | — |
| 270 mg TQS-168 SDD | | (245.4) | | (137.3) | |
| Powder for Oral Suspension | TQS-621 | 62 | 2.62 | 4950 | 5.92 |
| in the Fasted State | | (96.0) | (69.3) | (97.1) | (32.8) |

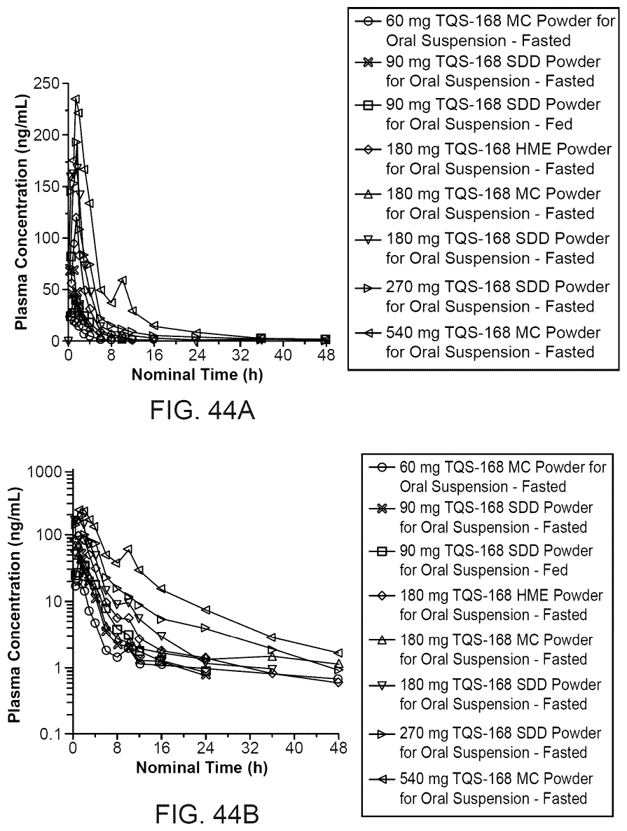

TQS-168 single ascending dose (SAD) PK profile: Cohort 3 Part 2 received Regimen D, 180 mg TQS-168 SDD powder for oral suspension in the fasted state and provided a TQS-168 Cₘₐₓ of 218 ng/mL (0.87 μM). Cohort 4 received Regimen F, 90 mg TQS-168 SDD powder for oral suspension in the fed state and provided a TQS-168 Cₘₐₓ of 47.1 ng/mL (0.19 μM). Cohort 5 received Regimen G, 90 mg TQS-168 SDD powder for oral suspension in the fasted state and provided a TQS-168 Cₘₐₓ of 111 ng/mL (0.44 μM). Cohort 6 received Regimen H, 270 mg TQS-168 SDD powder for oral suspension in the fasted state and provided the highest TQS-168 Cₘₐₓ of 237 ng/mL (0.95 μM). Results are plotted in FIG. 44. The recipients of Regimen H also displayed higher exposure (AUC₀₋₂₄=736 hr*ng/mL, AUC_{0-inf}=836 hr*ng/mL) than recipients of Regimen D (AUC₀₋₂₄=608 hr*ng/mL, AUC_{0-inf}=621 hr*ng/mL), Regimen G (AUC₀₋₂₄=192 hr*ng/mL, AUC_{0-inf}=195 hr*ng/mL) and Regimen F (AUC₀₋₂₄=168 hr*ng/mL, AUC_{0-inf}=171 hr*ng/mL).

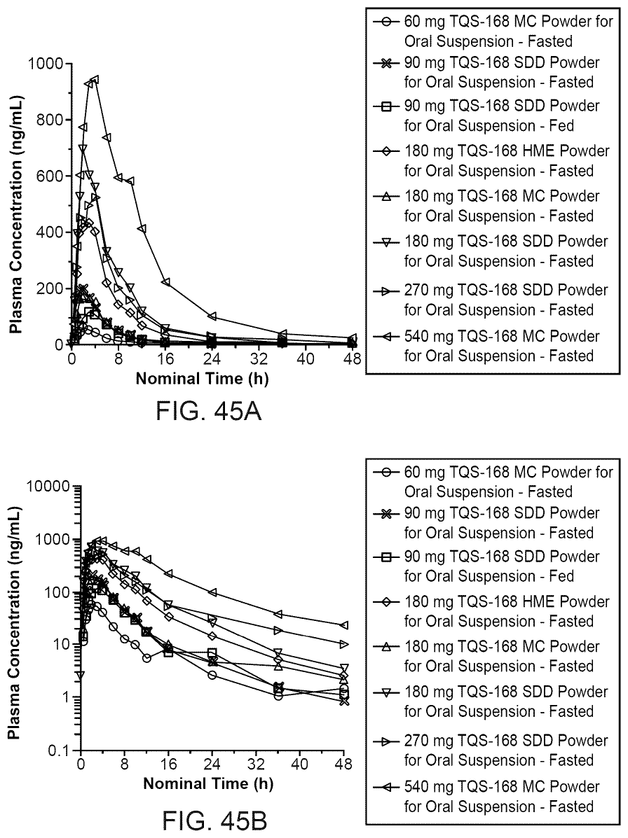

Metabolite TQS-621 PK profile: Regimen D (180 mg TQS 168) provided higher metabolite TQS-621 Cₘₐₓ (742 ng/mL, 2.79 μM) than Regimen H (621 ng/mL, 2.33 μM), Regimen G (214 ng/mL, 0.80 μM) and Regimen F (122 ng/mL, 0.46 μM). Results are plotted in FIG. 45. Similarly, recipients of Regimen D also displayed higher metabolite exposure (AUC₀₋₂₄=5110 hr*ng/mL, AUC_{0-inf}=5170 hr*ng/mL) than recipients of Regimen H (AUC₀₋₂₄=4250 hr*ng/mL, AUC_{0-inf}=4950 hr*ng/mL), Regimen G (AUC₀₋₂₄=1170 hr*ng/mL, AUC_{0-inf}=1200 hr*ng/mL) and Regimen F (AUC₀₋₂₄=848 hr*ng/mL, AUC_{0-inf}=901 hr*ng/mL).

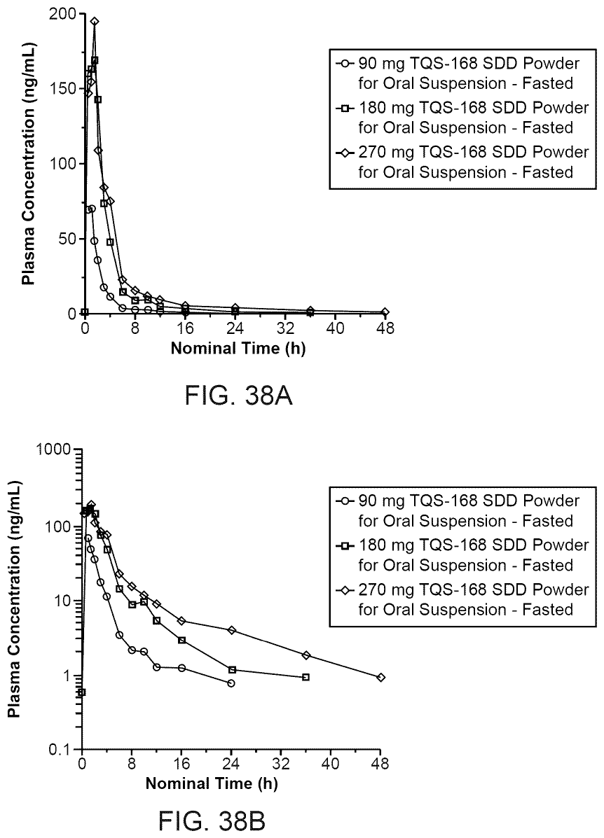

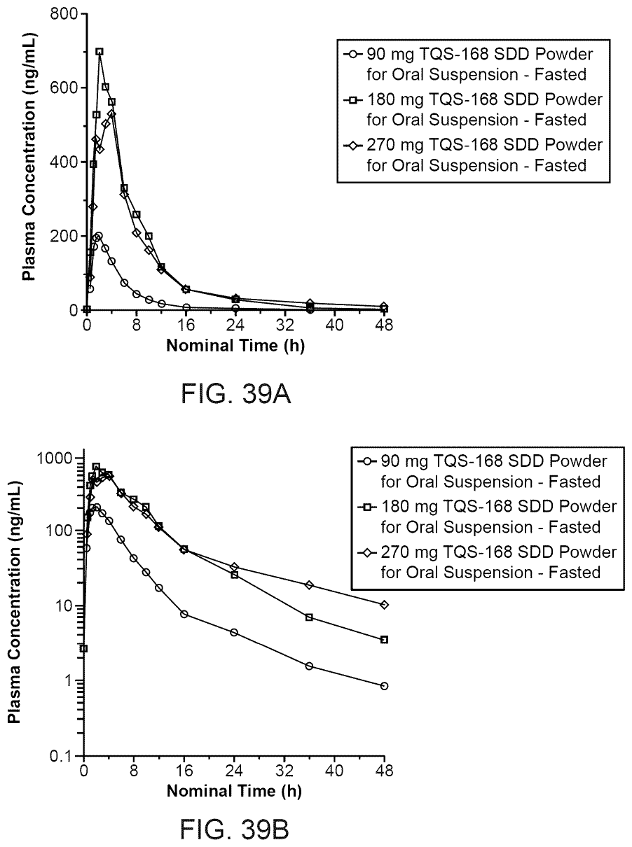

Comparing administration of 270 mg TQS-168 SDD powder for oral suspension in the fasted state (Regimen H) to administration of 90 mg TQS-168 SDD powder for oral suspension also in the fasted state (Regimen G) revealed that the 3-fold increase in dose resulted in 2.14- and 4.29-fold increases in TQS-168 Cₘₐₓ and AUC(0-inf), and 2.90- and 4.13-fold increases in TQS-621 Cₘₐₓ and AUC(0-inf). See FIGS. 38-39.

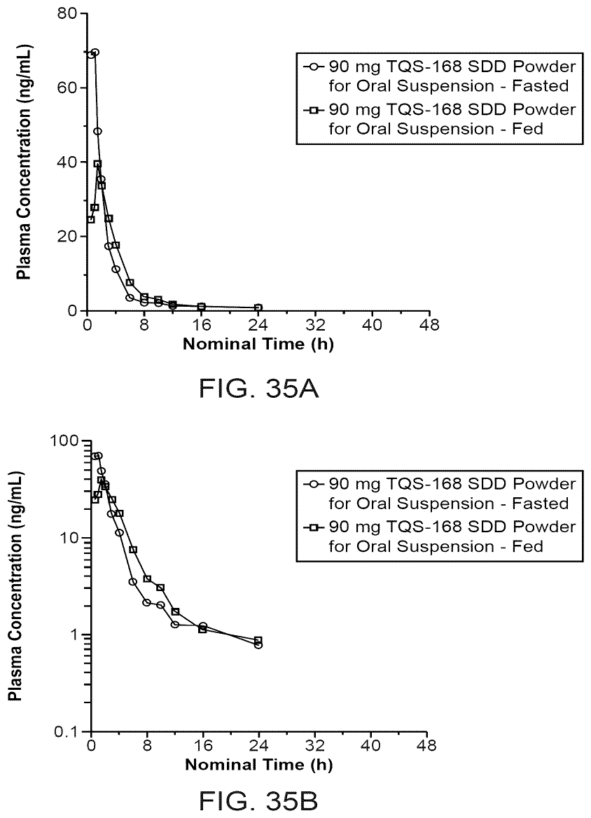

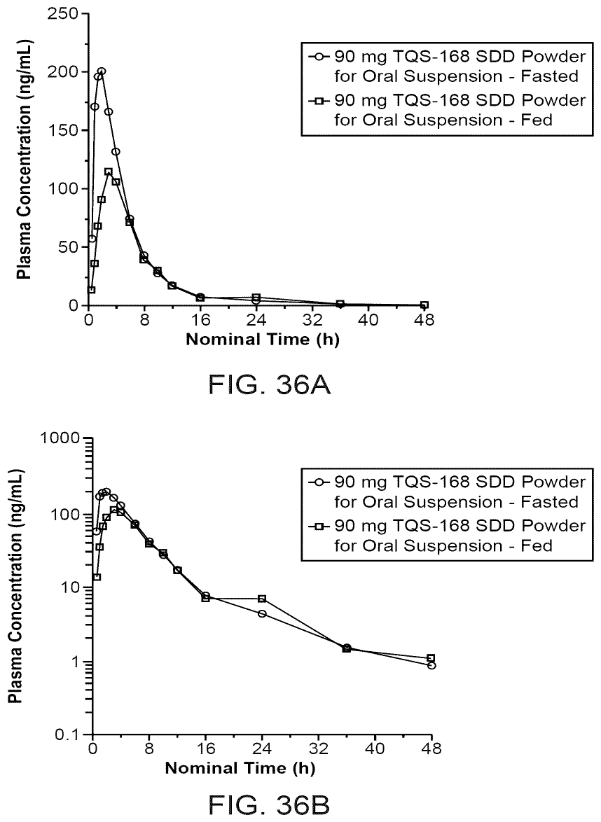

In a notable comparison, administration of 90 mg TQS-168 SDD powder for oral suspension in the fasted state (Regimen G) versus the same in the fed state (Regimen F), administration in the fasted state revealed increases of approximately 136% and 14% TQS-168 Cₘₐₓ and AUC(0-inf), and 75% and 33% TQS-621 Cₘₐₓ and AUC(0-inf). See FIGS. 35-36.

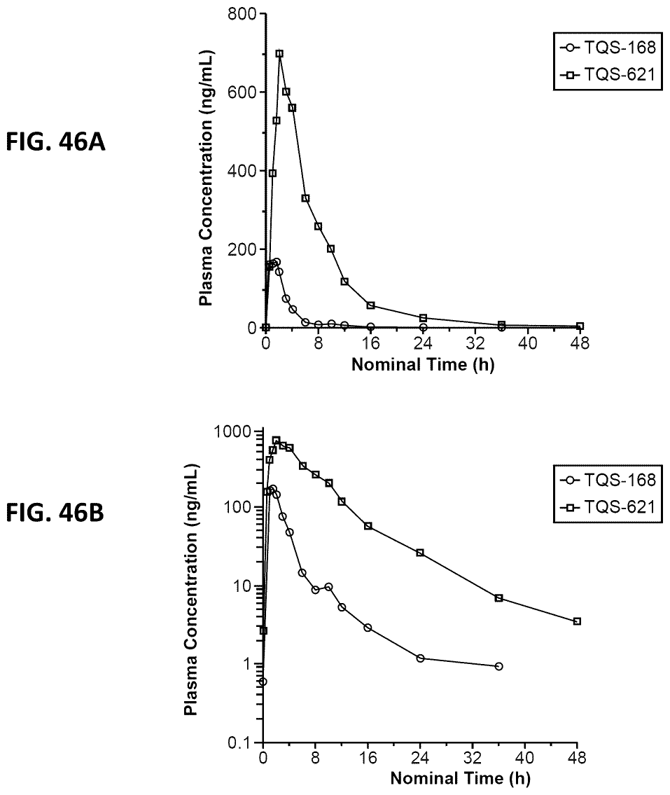

Comparing administration of 270 mg TQS-168 SDD powder for oral suspension in the fasted state (Regimen H) to administration of 180 mg TQS-168 also in the fasted state (Regimen D) revealed an increase of approximately 8.7% and 26% TQS-168 Cₘₐₓ and AUC(0-inf), but a decrease of approximately 16% and 4.3% TQS-621 Cₘₐₓ and AUC(0-inf). See FIGS. 38 and 46.

Following oral administration of Regimen D, 180 mg TQS 168 SDD in the fasted state, maximum plasma TQS-168 concentrations occurred between 0.5 and 2 hours post-dose, with a median Tₘₐₓ of 1 hour post-dose. Maximum plasma metabolite TQS-621 concentrations occurred between 1.5-3.0 hours post dose, with a median Tₘₐₓ of 2 hours post-dose. See FIGS. 46A-B.

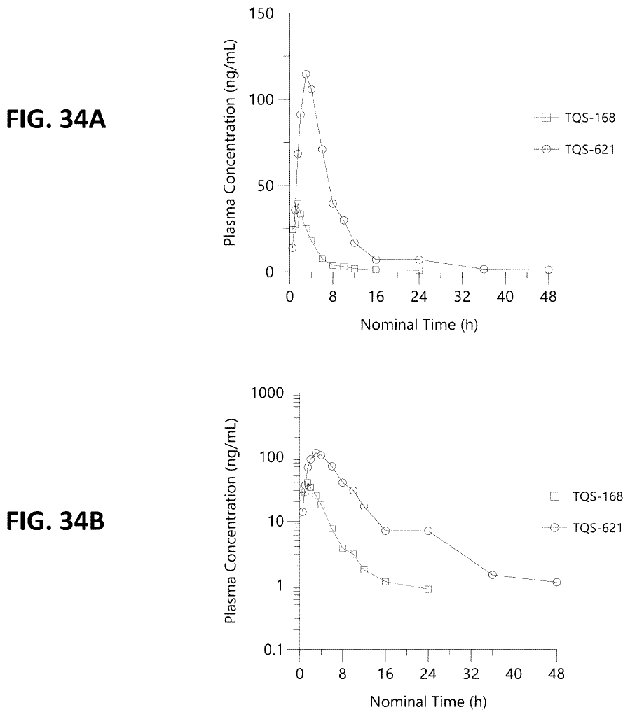

Following oral administration of Regimen F, 90 mg TQS-168 SDD in the fed state, maximum plasma TQS-168 concentrations occurred between 0.5 and 3 hours post-dose, with a median Tₘₐₓ of 1.5 hour post-dose. Maximum plasma metabolite TQS-621 concentrations occurred between 1.5-6.0 hours post dose, with a median Tₘₐₓ of 3.5 hours post-dose. See FIGS. 34A-B

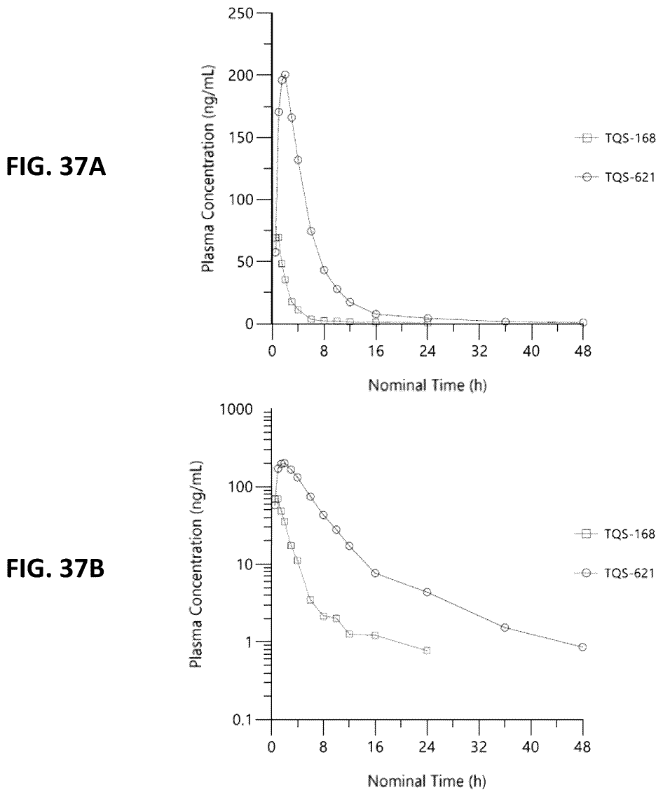

Following oral administration of Regimen G, 90 mg TQS-168 SDD in the fasted state, the maximum plasma concentrations of TQS-168 occurred between 0.5 and 2.0 hours post-dose. A median Tₘₐₓ of 0.5 hour post-dose was observed. Maximum plasma metabolite TQS-621 concentrations occurred between 1.0 and 3.0 hours post dose, with a median Tₘₐₓ of 1.25 hours post-dose. See FIGS. 37A-B.

Following oral administration of Regimen H, 270 mg TQS-168 SDD in the fasted state, the maximum plasma concentration of TQS-168 occurred between 0.5 and 4 hours post-dose, with a median Tₘₐₓ of 1.50 hours post-dose. Metabolite TQS-621 displayed maximum plasma concentrations between 1.5 and 4 hours post dose, with a median Tₘₐₓ of 2 hours post-dose. See FIGS. 38-39.

The terminal half-life (T_{1/2}) of TQS-168 following the 90 mg fed state, 90 mg fasted state, 180 mg fasted state and 270 mg fasted state TQS-168 SDD was dose-dependent at 3.52, 4.85, 5.64 and 10.4 hours, respectively. The terminal half-life of TQS-168 metabolite TQS-621 at dose 90 mg fed state, 90 mg fasted state, 180 mg fasted state and 270 mg in the fasted state was 5.17, 4.79, 7.12 and 10.4 hours, respectively. It is noted that for all doses of TQS-168, plasma concentrations of TQS-168 were quantifiable from 0.5 hours post-dose and remained quantifiable up to the final sample time point of 48 hours post-dose. Concentrations of TQS-621 were also quantifiable from 0.5 hours post-dose and remained quantifiable up to the final sampling time point of 48 hours post-dose.

### 5.16.6. HME Formulation and Oral Suspension of the Same

### 5.16.6.1 HME Preparation of a Hot-Melt Extrusion (HME) Formulation of 2-(4-tert-butylphenyl)-1H-benzimidazole (Compound 1)

A hot-melt extrusion (HME) formulation of 2-(4-tert-butylphenyl)-1H-benzimidazole (compound of formula I; TQS-168) having the composition set out in Table 24 was prepared as set out below.

TABLE 25
Hot-melt extrusion (HME) formulation of compound of formula I

| | | Amount | Amount (mg) |
|---|---|---|---|
| | | (mg) for 60 | for 1000 mg |
| Ingredient | % w/w | mg dose | dose |
| Compound of Formula I | 25 | 60 | 1000 |
| Povidone (Kollidon 17PF) | 25 | 60 | 1000 |
| Copovidone (Kollidon VA64) | 25 | 60 | 1000 |
| polyvinyl caprolactam-polyvinyl | 25 | 60 | 1000 |
| acetate-polyethylene glycol | | | |
| graft co-polymer (Soluplus) | | | |
| Total | 100 | 240 | 4000 |

The required quantities of compound 1, polyvinyl caprolactam-polyvinyl acetate-polyethylene glycol graft co-polymer (Soluplus), povidone (Kollidon 17PF) and copovidone (Kollidon VA64) according to Table 25 were weighed, passed through an 850 μm sieve and transferred to a 2 L blender shell. The resultant blender shell was secured in a blender (Pharmatech blender) and blended for 20 minutes then added to a double polyethylene (PE) bag (“compound of formula I blend”) and transferred to the HIME containment system. A chiller unit was connected to the HME and once the chiller temperature reached 15° C. the extrusion process was commenced using the parameters outlined in Table 26. The compound of formula I blend was added to the feeder to fill approximately ¾ of the feeder, and the extrudate was collected and discarded for approximately the first 5 minutes of the extrusion process. The feeder was refilled to maintain approximately 50 volume in the feeder throughout the process, and extrusion was continued until all the compound of formula I blend was extruded and collected (“compound of formula I HIME extrudate”).

TABLE 26
HME parameters

| Feed rate | 0.110-0.175 kg/hour | | | | | | | |
|---|---|---|---|---|---|---|---|---|
| Temperature | Zone 2 | Zone 3 | Zone 4 | Zone 5 | Zone 6 | Zone 7 | Zone 8 | Die |
| (all zones) | 50-70° C. | 80-170° C. | 200-220° C. | 220-240° C. | 220-240° C. | 220-240° C. | 220-240° C. | 170-195° C. |

| | | |
|---|---|---|
| Screw speed | 30 | rpm |
| Die Dimension | 1.0 | mm |
| Chiller temperature | 15° | C. |

The collected compound of formula I HME extrudate was added to a U5 Quadro mill (set up with a screen size of 457 (mm) and an impeller speed of 5000 RPM), until all extrudate had passed the 457 mm screen to obtain milled granules of compound of formula I. The milled granules of compound 1 were then sieved using a 300 micron sieve and transferred into a blender shell (Pharmatech 2 L blender shell). The resultant blender shell was secured in a blender (Pharmatech blender), blended for 5 minutes, and collected.

### 5.16.6.2 Oral Suspension of a Hot-Melt Extrusion (HME) Formulation of 2-(4-tert-butylphenyl)-1H-benzimidazole (Compound of Formula I)

The hot-melt extrusion (HME) of 2-(4-tert-butylphenyl)-1H-benzimidazole (compound of formula I) having the composition set out in Table 25 (e.g., from 60-1000 mg) was reconstituted as an oral suspension in 100 mL of vehicle Ora-Blend SF® (purified water, sucrose, glycerin, sorbitol, flavoring, microcrystalline cellulose, sodium carboxymethylcellulose, xanthan gum, carrageenan, citric acid, sodium phosphate, simethicone, potassium sorbate and methylparaben), a commercially available oral suspending vehicle manufactured by Perrigo Pharmaceuticals.

### 5.16.7. HME Results

Healthy male subjects were administered a single oral dose of TQS-168 HME powder oral suspension at 180 mg in the fasted state (Regimen E). Plasma concentrations of TQS-168 and metabolite TQS-621 were measured over time, and key pharmacokinetic parameters determined.

Table 27 and Table 28 present the geometric mean of TQS-168 and metabolite TQS-621 key pharmacokinetic parameters in the subjects following oral administration of TQS-168 SDD formulation.

TABLE 27
Geometric Mean (CV %) key pharmacokinetic parameters HME formulation

| Treatment | | Tmax* | Cmax | AUC(0-24) | AUC(0-last) | AUC(0-inf) | T_{1/2} |
|---|---|---|---|---|---|---|---|
| Part 1 | Analyte | (h) | (ng/mL) | (ng · h/mL) | (ng · h/mL) | (ng · h/mL) | (h) |
| Cohort 3 Period 2 | TQS-168 | 1.50 | 123 | 358 | 360 | 370 | 7.25 |
| Regimen E | | (1.00-1.50) | (66.9) | (59.5) | (60.9) | (60.2) | (54.4) |
| 180 mg TQS-168 | TQS-621 | 2.00 | 481 | 3090 | 3250 | 3290 | 9.17 |
| HME Powder for | | (1.50-4.00) | (50.8) | (42.8) | (42.2) | (42.2) | (32.3) |
| Oral Suspension | | | | | | | |
| in the fasted state | | | | | | | |

TABLE 28
Geometric Mean (CV %) key pharmacokinetic parameters HME formulation

| Treatment | | Cmax | MPR | AUC(0-inf) | MPR |
|---|---|---|---|---|---|
| Part 1 | Analyte | (ng/mL) | Cmax | (ng · h/mL) | AUC(0-inf) |
| Cohort 3 Period 2 | TQS-168 | 123 | — | 370 | — |
| Regimen E 180 mg | | (66.9) | | (60.2) | |
| TQS-168 HME Powder | TQS-621 | 481 | 3.90 | 3290 | 8.88 |
| for Oral Suspension | | (50.8) | (33.9) | (42.2) | (30.2) |
| in the fasted state | | | | | |

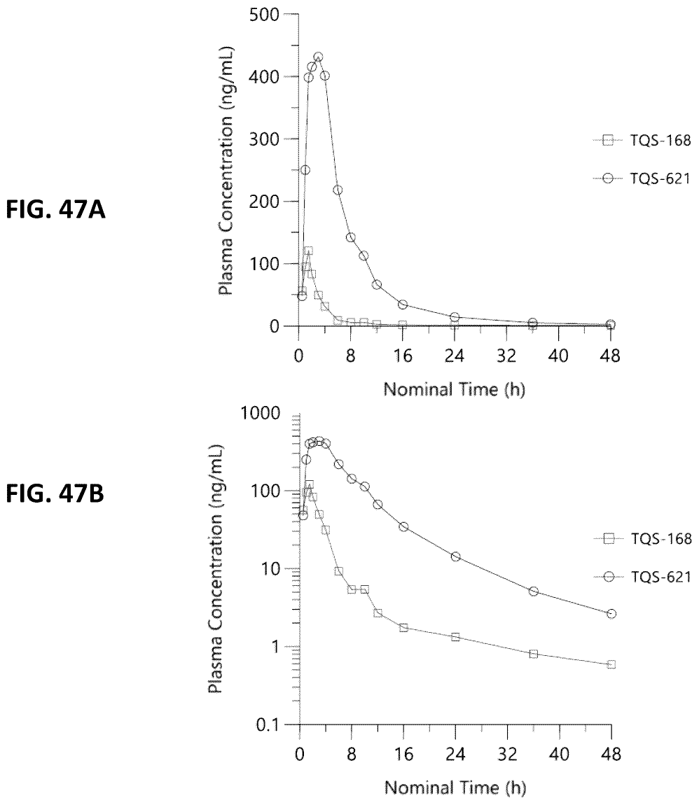

TQS-168 single ascending dose (SAD) PK profile: Cohort 3 Period 2 received Regimen E, 180 mg TQS-168 HME powder in oral suspension in the fasted state. The single dose provided a TQS-168 Cₘₐₓ of 123 ng/mL (0.49 μM) and an AUC0-24 of 358 hr*ng/mL. Data are plotted in FIGS. 47A-B.

Metabolite TQS-621 PK profile: Regimen E provided metabolite TQS-621 Cₘₐₓ of 481 ng/mL (1.81 μM) and AUC₀₋₂₄ of 3090 hr*ng/mL. Illustrated in FIGS. 47A-B.

### 5.16.8. Comparison of MC Vs SDD Vs HME PK Results

Under Part 1 of this treatment, subjects received 180 mg TQS-168 in the following formulations in the fasted state: methyl cellulose (MC), spray dried dispersion (SDD) powder in oral suspension, or hot melt extrusion (HMNE) powder in oral suspension. Plasma concentrations of TQS-168 and metabolite TQS-621 were measured over time, and key pharmacokinetic parameters determined as previously shown. For convenience, Tables 29-31 compare the results. See FIG. 45A-B.

TABLE 29
Geometric Mean (CV %) key pharmacokinetic parameters MC, SDD and HME formulation

| Treatment | | Tmax* | Cmax | AUC(0-24) | AUC(0-last) | AUC(0-inf) | T_{1/2} |
|---|---|---|---|---|---|---|---|
| Part 1 | Analyte | (h) | (ng/mL) | (ng · h/mL) | (ng · h/mL) | (ng · h/mL) | (h) |
| Cohort 2 | TQS-168 | 1.00 | 53.1 | 163 | 168 | 180 | 7.36 |
| Regimen B | | (1.00-4.00) | (55.0) | (46.3) | (51.8) | (53.1) | (75.5) |
| 180 mg | TQS-621 | 1.50 | 199 | 1210 | 1320 | 1350 | 9.10 |
| TQS-168 | | (1.00-4.00) | (33.5) | (42.2) | (45.9) | (45.1) | (85.2) |
| MC Powder | | | | | | | |
| for Oral | | | | | | | |
| Suspension | | | | | | | |
| Cohort 3 | TQS-168 | 1.00 | 218 | 608 | 611 | 621 | 5.64 |
| (Regimen D) | | (0.50-2.00) | (77.2) | (78.5) | (78.8) | (77.4) | (32.4) |
| 180 mg | TQS-621 | 2.00 | 742 | 4860 | 5110 | 5170 | 7.12 |
| TQS-168 | | (1.50-3.00) | (39.9) | (37.3) | (35.8) | (35.2) | (32.4) |
| SDD | | | | | | | |
| Powder for | | | | | | | |
| Oral | | | | | | | |
| Suspension | | | | | | | |
| Cohort 3 | TQS-168 | 1.50 | 123 | 358 | 360 | 370 | 7.25 |
| Regimen E | | (1.00-1.50) | (66.9) | (59.5) | (60.9) | (60.2) | (54.4) |
| 180 mg | TQS-621 | 2.00 | 481 | 3090 | 3250 | 3290 | 9.17 |
| TQS-168 | | (1.50-4.00) | (50.8) | (42.8) | (42.2) | (42.2) | (32.3) |
| HME | | | | | | | |
| Powder for | | | | | | | |
| Oral | | | | | | | |
| Suspension | | | | | | | |

*Median (min-max)

TABLE 30
Geometric Mean (CV %) key pharmacokinetic
parameters MC, SDD and HME formulation

| Treatment | | Cmax | MPR | AUC(0-inf) | MPR |
|---|---|---|---|---|---|
| Part 1 | Analyte | (ng/mL) | Cmax | (ng · h/mL) | AUC(0-inf) |
| Cohort 2 | TQS-168 | 53.1 | — | 180 | — |
| (Regimen B) | | (55.0) | | (53.1) | |
| 180 mg TQS-168 | TQS-621 | 199 | 3.74 | 1350 | 7.52 |
| MC Powder for | | (33.5) | (65.1) | (45.1) | (29.4) |
| Oral Suspension | | | | | |
| Cohort 3 | TQS-168 | 218 | — | 621 | — |
| (Regimen D) | | (77.2) | | (77.4) | |
| 180 mg TQS-168 | TQS-621 | 742 | 3.41 | 5170 | 8.32 |
| SDD Powder for | | (39.9) | (52.9) | (35.2) | (42.4) |
| Oral Suspension | | | | | |
| Cohort 3 | TQS-168 | 123 | — | 370 | — |
| (Regimen E) | | (66.9) | | (60.2) | |
| 180 mg TQS-168 | TQS-621 | 481 | 3.90 | 3290 | 8.88 |
| HME Powder for | | (50.8) | (33.9) | (42.2) | (30.2) |
| Oral Suspension | | | | | |

TABLE 31
Geometric mean (geometric CV %) relative bioavailability of SDD powder and
HME powder for oral suspension following oral administration of TQS-168

| | | Frel | Frel | Frel | Frel | Frel | Frel |
|---|---|---|---|---|---|---|---|
| Treatment | | Cmax/D^{a} | AUC(0-last)/D^{a} | AUC(0-inf)/D^{a} | Cmax/^{b} | AUC(0-last)/^{b} | AUC(0-inf)/^{b} |
| Part 1 | Analyte | (%) | (%) | (%) | (%) | (%) | (%) |
| Cohort 3 | TQS-168 | 218 | 120 | 118 | — | — | — |
| (Regimen D) | | (84.9) | (80.9) | (74.6) | | | |
| 180 mg TQS-168 | TQS-621 | 203 | 122 | 119 | | | |
| SDD Powder for | | (18.8) | (56.5) | (53.3) | | | |
| Oral Suspension | | | | | | | |
| Cohort 3 | TQS-168 | 124 | 70.9 | 70.4 | 56.6 | 58.9 | 59.6 |
| (Regimen E) | | (82.9) | (67.0) | (60.7) | (33.9) | (30.6) | (30.0) |
| 180 mg TQS-168 | TQS-621 | 132 | 77.7 | 75.7 | 64.9 | 63.5 | 63.6 |
| HME Powder for | | (23.2) | (50.1) | (46.3) | (16.4) | (13.8) | (13.9) |
| Oral Suspension | | | | | | | |

^{a}dose normalized comparison to Regimen C: 540 mg TQS-168 MC Powder for Oral Suspension
^{b}comparison to Regimen D 180 mg TQS-168 SDD Powder for Oral Suspension

Subjects administered 180 mg TQS-168 MC powder for oral suspension (Regimen B) displayed plasma concentrations of TQS-168 quantifiable from 0.5 hours post-dose that remained quantifiable up to between 10 and 48 hours post-dose. Concentrations of TQS-621 were also quantifiable from 0.5 hours post-dose and remained quantifiable up to between 24 and 48 hours post-dose.

Maximum plasma TQS-168 concentrations occurred between 1 and 4 hours post dose, with a median Tₘₐₓ of 1 hour post-dose. The resultant T½ was 7.36 hours. Geometric mean (CV %) Cₘₐₓ and AUC(0-inf) values were 53.1 ng/mL (55%) and 180 ng*h/mL (53.1%) respectively.

Maximum plasma TQS-621 concentrations occurred between 1 and 4 hours post dose, with a median Tₘₐₓ of 1.5 hours post-dose. The resultant geometric mean T½ was 9.10 hours. Geometric Mean (CV %) Cₘₐₓ and AUC(0-inf) values were 199 ng*hr/mL (33.5%) and 1350 ng*h/mL (45.1) respectively.

Following administration of 180 mg TQS-168 SDD powder for oral suspension (Regimen D) plasma concentrations of TQS-168 were quantifiable from 0.5 hours post-dose and remained quantifiable up to between 24 and 36 hours post-dose. Note, one subject displayed plasma concentrations of TQS-168 quantifiable at pre-dose as a result of some carry-over from the previous dosing regimen. Concentration of TQS-621 was quantifiable from pre-dose in all subjects as a result of some carry-over from the previous dosing regimen. It remained quantifiable up to the final sampling time point of 48 hours post-dose (Day 5). Note all quantifiable pre-dose concentrations were less than 5% of Cₘₐₓ.

Maximum plasma TQS-168 concentrations occurred between 0.5 and 2 hours post-dose, with a median Tmax of 1 hour post-dose. The resultant geometric mean T_{1/2} was 5.64 hours. Maximum plasma TQS-621 concentrations occurred between 1.5 and 3 hours post-dose, with a median Tmax of 2 hours post dose. The resultant geometric mean T_{1/2} was 7.12 hours.

The geometric mean Cmax, AUC(0-last) and AUC(0-inf) of TQS-168 following administration of 180 mg SDD powder for oral suspension compared to its MC counterpart (Regimen B) resulted in a 4.11-, 3.64-, and 3.45-fold increase respectively. The geometric mean Cmax, AUC(0-last) and AUC(0-inf) of TQS-621 following administration of 180 mg SDD powder for oral suspension compared to its MC counterpart (Regimen B) resulted in a 3.73-, 3.87- and 3.83-fold increase respectively.

Following administration of 180 mg TQS-168 HME powder for oral suspension (Regimen E), subjects displayed plasma concentrations of TQS-168 quantifiable from 0.5 hours post-dose and remained quantifiable up to between 16 and the final sampling time point of 48 hours post-dose (Day 5). Concentrations of TQS-621 were also quantifiable from 0.5 hours post-dose and remained quantifiable up to the final sampling time point of 48 hours post-dose in all subjects.

Maximum plasma TQS-168 concentrations occurred between 1 and 1.5 hours post-dose, with a median Tmax of 1.5 hour post-dose. The resultant geometric mean T½ was 7.25 hours. Maximum plasma TQS-621 concentrations occurred between 1.5 and 4 hours post-dose, with a median Tmax of 2 hour post-dose. The resultant geometric mean T_{1/2} was 9.17 hours.

The geometric mean (geometric CV %) relative bioavailability of TQS-168 following administration of 180 mg HME (Regimen E), based on Cₘₐₓ, AUC(0-last) and AUC(0-inf), was 56.6% (33.9%), 58.9% (30.6) and 59.6% (30.0%), when compared to administration of 180 mg SDD (Regimen D). The geometric mean (geometric CV %) relative bioavailability of metabolite TQS-621 following administration of 180 mg TQS-168 HME, based on Cₘₐₓ, AUC(0-last) and AUC(0-inf), was 64.9% (16.4%), 63.5% (13.8) and 63.6% (13.9)%), when compared to administration of 180 mg SDD. The geometric mean Cₘₐₓ, AUC(0-last) and AUC(0-inf) of TQS-168 following administration of 180 mg HME (Regimen E) compared to its MC counterpart (Regimen B) resulted in a 2.32-, 2.14-, and 2.06-fold increase respectively.

The geometric mean Cmax, AUC(0-last) and AUC(0-inf) of TQS-621 following administration of 180 mg HME compared to its MC counterpart resulted in a 2.42-, 2.46-, and 2.44-fold increase respectively.

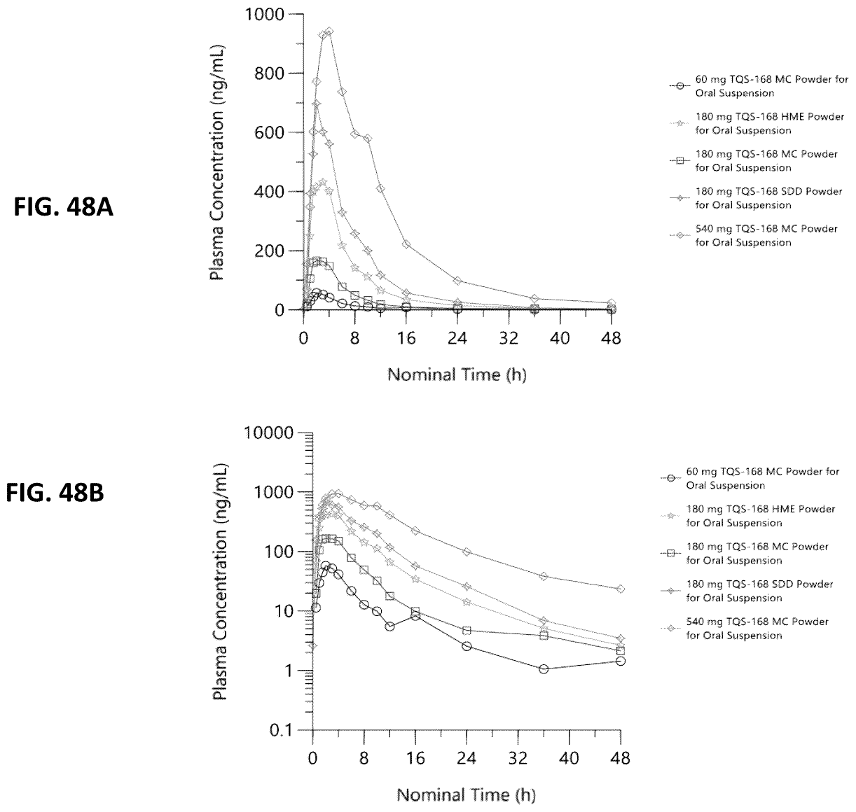

In summary the SDD formulation showed significant improvement in exposure over the MC and the HME formulations of the same TQS-168 dosage, as well as in exposure of metabolite TQS-621. See FIG. 48A-B.

### 5.17. Example 17—Part 2: Multiple Dose Phase 1 Trial—Double-Blind Randomized Study of Subjects Receiving Multiple Doses of TQS-168 or Placebo

### 5.17.1. Trial Design

Part 2 is a double-blind, randomized, placebo-controlled clinical study that was conducted to characterize and compare the pharmacokinetic (PK) profile of TQS-168 and its metabolite TQS-621 following multiple doses of a TQS-168 spray dried dispersion (SDD) powder for oral suspension formulation in healthy subjects. See Table 32 for description of dose regimens.

TABLE 32
Description of Regimens

| | Period and | | | | | |
|---|---|---|---|---|---|---|
| | Frequency | | | | | Route of |
| Cohort | (Dosing Day) | Regimen | IMP | Dose | | Administration |
| 1 | 1-7 | I | TQS-168 SDD | 120 | mg | Oral, Fasted |
| | QD | | Powder for Oral | | | |
| | | | Suspension | | | |
| | | | formulation | | | |
| 2 | 1-7 | J | TQS-168 SDD | 90 | mg | Oral, Fed |
| | QD | | Powder for Oral | | | |
| | | | Suspension | | | |
| | | | formulation | | | |
| 3 | 1-7 | K | TQS-168 SDD | 300 | mg | Oral, Fed |
| | QD | | Powder for Oral | | | |
| | | | Suspension | | | |
| | | | formulation | | | |

IMP: investigational medicinal product;
N/A: not applicable.

This randomized, double-blind, placebo-controlled phase 1 multiple dose trial was conducted in healthy male subjects aged 18 to 55 years with body mass index (BMI) 18.0 to 32.0 kg/m² as measured at screening. Subjects all weighed at least 55 kg at screening. Key criteria for exclusion were subjects with evidence of current SARS-CoV-2 infection, clinical manifestation of significant cardiovascular, renal, hepatic, dermatological, chronic respiratory or gastrointestinal disease, or aspartate aminotransferase (AST) or alanine aminotransferase (ALT)>1.5× the upper limit of normal (ULN). Subjects were recruited at a single site in the United Kingdom. Each patient provided written informed consent.

The trial was performed in 3 cohorts. All subjects were admitted in the morning on the day before dosing (Day −1) and remained onsite until 48 hours post-final dose (Day 9). The screening period was 4 weeks. After confirming eligibility, subjects were randomly assigned to receive either the IMP (TQS-168) or placebo treatment. Subjects were dosed with the IMP or placebo on the morning of Days 1 to 7 (approximately 24 hours apart). Administration was performed in either the fasted state (Regimen I) following an overnight fast (minimum of 10 hours), or the fed state (following a standard pre-dose or high fat meal given 30 minutes before dosing). Safety was continually assessed throughout the trial by monitoring adverse events and concomitant medication use, electrocardiograms (ECGs), vital signs, laboratory safety assessments and physical examinations. Blood samples for pharmacokinetic assessments were collected from each subject prior from Day −1, (≤1 hr) to each dose, and at intervals throughout the study until 48 hours post final dose as applicable.

Assessment of Pharmacokinetics: Blood samples for plasma PK analysis were collected at regular time intervals. Venous blood samples were collected from the subjects by a trained member of the clinical team. Pre-dose samples were taken ≤1 h before dosing. Timestamp 0 to 1 hour post-dose samples were taken within ±2 minutes of the nominal post-dose sampling time. Timestamp 1.5 to 12 hour post-dose samples were taken within ±10 min of the nominal post-dose sampling time. Timestamp 16 to 48 hour post-dose samples were taken within ±30 minutes of the nominal post-dose sampling time. Samples were collected into appropriate containers and were processed to isolate plasma. PK analysis were carried out on plasma samples using validated bioanalytical methods.

Statistical Analyses: The sample sizes for the study were chosen based on practical considerations and experience from previous studies of a similar design. The numbers of subjects in each cohort (group) were considered to be adequate to assess the main objectives of each study. Pharmacokinetic parameters were determined by non-compartmental techniques using WinNonlin software version 8.0 or higher (Certara USA. Inc., USA). All data were listed and summarized by subject group using descriptive statistics. All statistical analyses were conducted using SAS version 9.4 or higher.

### 5.17.2. Spray-Dried Dispersion (SDD) Formulation and Oral Suspension of the Same

### 5.17.2.1 Preparation of a SDD formulation of 2-(4-tert-butylphenyl)-1H-benzimidazole (compound of formula I)

A spray-dried dispersion (SDD) of 2-(4-tert-butylphenyl)-1H-benzimidazole (compound of formula I) having the composition set out in Table 20 was prepared by spray drying a feedstock formulation set out in Table 21.

TABLE 20
Spray Dried Dispersion (SDD) Formulation
of compound of formula I

| | | Amount |
|---|---|---|
| Ingredient | % w/w | (g) |
| Compound of formula I | 30 | 45.0 |
| polyvinyl caprolactam-polyvinyl acetate-polyethylene | 40 | 60.0 |
| glycol graft co-polymer (Soluplus) | | |
| amorphous silicon dioxide (Syloid ® 244 FP) | 30 | 45.0 |
| Total | 100 | 150 |

TABLE 21
Spray Drying Feedstock Formulation

| | | Amount |
|---|---|---|
| Ingredient | % w/w | (g) |
| Compound of formula I | 2.3 | 45.0 |
| polyvinyl caprolactam-polyvinyl acetate-polyethylene | 3.1 | 60.0 |
| glycol graft co-polymer (Soluplus) | | |
| amorphous silicon dioxide (Syloid ® 244 FP) | 2.3 | 45.0 |
| 2-propanol | 92.3 | 1797.1 |
| Total | 100 | 1947.1 |

### 5.17.2.2 Manufacturing Procedure for Spray Drying Feedstock Formulation

Compound of formula I (45.0 g) was slowly added to 2-propanol (1791.1 g) with stirring, placed under a homogenizer (Silverson SL2 homogenizer) and stirred for 5 minutes or more until Compound of formula I was fully dissolved. The reaction mixture was then removed from the homogenizer and polyvinyl caprolactam-polyvinyl acetate-polyethylene glycol graft co-polymer (Soluplus) (60.0 g) was slowly added with stirring, placed back under the homogenizer and stirred for 10 minutes or more until the Soluplus was fully dissolved. The reaction mixture was then removed from the homogenizer and amorphous silicon dioxide (Syloid® 244 FP) was slowly added with stirring, placed back under the homogenizer and stirred for an additional 15 minutes or more until the amorphous silicon dioxide was fully dispersed. The resulting suspension is referred to herein as the “Feedstock Formulation.”

### 5.17.2.3 Manufacturing Procedure for Spray Dried Dispersion (SDD) Formulation of Compound of Formula I

The spray dryer unit (ProCepT 4M8 Spray Dryer) was set up with a compressed air supply. Once the outlet temperature stabilized, the feed pump was initiated and 2-propanol (blank solution) was sprayed through the nozzle as a fine spray into the collection chamber. The spray dryer parameters were adjusted to achieve a feed rate within the range set out in Table 22 below.

The feedstock formulation was stirred under a homogenizer at a speed appropriate to maintain a homogenous dispersion without generating bubbles. The feedstock formulation was then sprayed through the nozzle as a fine spray into the collection chamber of the spray dryer unit (ProCepT 4M8 Spray Dryer, using parameters as set up with the blank solution and outlined in Table 3) where the solvent was evaporated quickly to generate particles containing compound of formula I polyvinyl caprolactam-polyvinyl acetate-polyethylene glycol graft co-polymer (Soluplus) and silicon dioxide (Syloid® 244 FP) (SDD formulation of compound of formula I). Once all the feedstock formulation had been sprayed and collected, the feedstock formulation was replaced with 2-propanol (blank solution) and sprayed through the nozzle of the spray dryer for 5 minutes or more to allow collection of any remaining “feedstock formulation” within the air stream.

TABLE 22
Target Process Parameters for Spray Dryer

| Process Parameter | Target | |
|---|---|---|
| Inlet air flow | 0.45 | m³/min |
| Inlet air temperature | 88° | C. |
| Suspension feed rate | 4-7 | g/min |
| Peristaltic tubing | 50513 Tygon MHLL ID 2.79 mm | |
| | (2-stop, purple-white) | |
| Nozzle diameter | 0.6 | mm |
| Nozzle cap | with cap | |
| Nozzle pressure | 2 | bar |
| Cyclone gas pressure | 0.3 | bar |

### 5.17.2.1 Oral Suspension of a Spray Dried Dispersion (SDD) Formulation of 2-(4-Tert-Butylphenyl)-1H-Benzimidazole (Compound of Formula I)

The spray-dried dispersion (SDD) of 2-(4-tert-butylphenyl)-1H-benzimidazole (compound of formula I) having the composition set out in Table 20 (e.g., from 60-1000 mg) was reconstituted as an oral suspension in 100 g of vehicle composed of PEG 300 (10 g), glycerol monocaprylocaprate (Capmul MCM, 0.40 mg) in sterile water for irrigation (q.s. to 100 g).

Regimen I: Subjects received an oral dose of 120 mg TQS-168 spray dried dispersion (SDD) powder in oral suspension, or placebo, once a day for 7 consecutive days in the fasted state.

Regimen J: Subjects received an oral dose of 90 mg TQS-168 spray dried dispersion (SDD) powder in oral suspension, or placebo, once a day for 7 consecutive days in the fed state. Subjects were provided a high fat breakfast on Day 1 and 7, and a standard breakfast on Days 2-6.

Regimen K: Subjects received an oral dose of 300 mg TQS-168 spray dried dispersion (SDD) powder in oral suspension, or placebo, once a day for 7 consecutive days in the fed state. Subjects were provided a high fat breakfast on Day 1 and 7, and a standard breakfast on Days 2-6.

### 5.17.3. Results

Healthy male subjects were administered multiple oral doses of either 120 mg TQS-168 spray dried dispersion (SDD) powder in the fasted state (Regimen I) or 90 mg TQS-168 SDD in the fed state (Regimen J). Plasma concentrations of TQS-168 and metabolite TQS-621 were measured over time, and key pharmacokinetic parameters determined.

Table 33 presents the geometric mean of TQS-168 and metabolite TQS-621 key pharmacokinetic parameters in the subjects following oral administration of TQS-168.

TABLE 33
Geometric Mean (CV %) key pharmacokinetic parameters

| | | | | | | | MPR | MPR | AR | AR |
|---|---|---|---|---|---|---|---|---|---|---|
| Treatment | | | Tmax* | Cmax | AUC(0-tau) | T_{1/2} | Cmax | AUC(0-tau) | Cmax | AUC |
| Part 2 | Analyte | Day | (h) | (ng/mL) | (ng · h/mL) | (h) | (N/A) | (N/A) | (N/A) | (N/A) |
| Cohort 1 | TOS-168 | 1 | 1.00 | 172 | 438 | — | — | — | — | — |
| Regimen I | | | (0.50-2.00) | (49.3) | (51.5) | | | | | |
| 120 mg | | 7 | 0.750 | 273 | 692 | 7.70 | — | — | 1.59 | 1.58 |
| TQS-168 | | | (0.500-1.50) | (105.8) | (89.8) | (39.0) | | | (88.1) | (51.6) |
| SDD | TQS-621 | 1 | 1.50 | 331 | 2270 | — | 1.93 | 5.19 | — | — |
| Powder for | | | (1.00-4.00) | (40.3) | (35.7) | | (32.7) | (26.1) | | |
| Oral | | 7 | 1.75 | 340 | 3320 | 10.2 | 1.24 | 4.79 | 1.03 | 1.46 |
| Suspension | | | (1.00-4.00) | (39.5) | (42.1) | (35.5) | (57.3) | (40.4) | (38.7) | (30.8) |
| QD in the | | | | | | | | | | |
| Fasted | | | | | | | | | | |
| State | | | | | | | | | | |
| Cohort 2 | TQS-168 | 1 | 3.00 | 47.4 | 223 | — | — | — | — | — |
| Regimen J | | | (1.50-4.00) | (27.4) | (30.9) | | | | | |
| 90 mg | | 7 | 3.00 | 48.8 | 227 | 4.46 | — | — | 1.03 | 1.24 |
| TQS-168 | | | (1.00-4.00) | (48.3) | (46.7) | (36.5) | | | (37.5) | (22.8) |
| SDD | TQS-621 | 1 | 4.00 | 189 | 1400 | — | 3.99 | 6.28 | — | — |
| Powder for | | | (4.00-6.00) | (30.1) | (42.6) | | (26.6) | (20.6) | | |
| Oral | | 7 | 5.00 | 218 | 2060 | 8.65 | 4.47 | 7.43 | 1.15 | 1.47 |
| Suspension | | | (4.00-6.00) | (37.6) | (54.8) | (10.3 | (14.9) | (10.7) | (12.4) | (18.0) |
| QD in the | | | | | | | | | | |
| Fed State | | | | | | | | | | |
| Cohort 3 | TQS-168 | 1 | 2.00 | 229 | 1210 | — | — | — | — | — |
| Regimen K | | | (1.50-4.00) | (38.3) | (55.1) | | | | | |
| 300 mg | | 7 | 4.00 | 400 | 2010 | 5.67 | — | — | 1.74 | 1.66 |
| TQS-168 | | | (0.50-4.00) | (79.3) | (88.3) | (52.2) | | | (54.6) | (29.5) |
| SDD | TQS-621 | 1 | 4.00 | 1000 | 9730 | — | 4.37 | 8.06 | — | — |
| Powder for | | | (4.00-10.00) | (24.6) | (39.9) | | (26.1) | (18.1) | | |
| Oral | | 7 | 5.00 | 1300 | 14900 | 7.29 | 3.26 | 7.41 | 1.30 | 1.53 |
| Suspension | | | (4.00-6.00) | (36.7) | (61.2) | (42.4) | (43.4) | (25.5) | (32.9) | (26.1) |
| QD in the | | | | | | | | | | |
| fed state | | | | | | | | | | |

*Median (min-max)
Tau = 24 h
N/A = not applicaple

TQS-168 Regimen I PK profile: Subjects in Cohort 1 received a single 120 mg TQS-168 SDD p.o. QD in the fasted state for seven consecutive days.

On Day 1, following a single administration of Regimen I, plasma concentrations of TQS-168 were quantifiable from 0.5 hours post-dose for all subjects, and remained quantifiable up to between 16 and 24 hours post-dose. Concentrations of TQS-621 on Day 1 were also quantifiable from 0.5 h post-dose, and remained quantifiable up to 24 hours post-dose in all subjects.

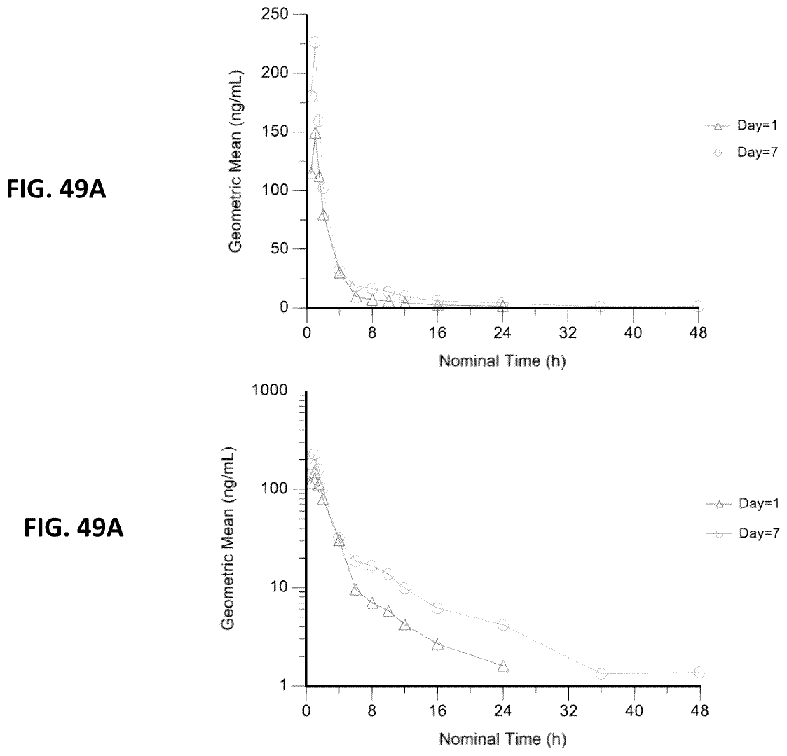

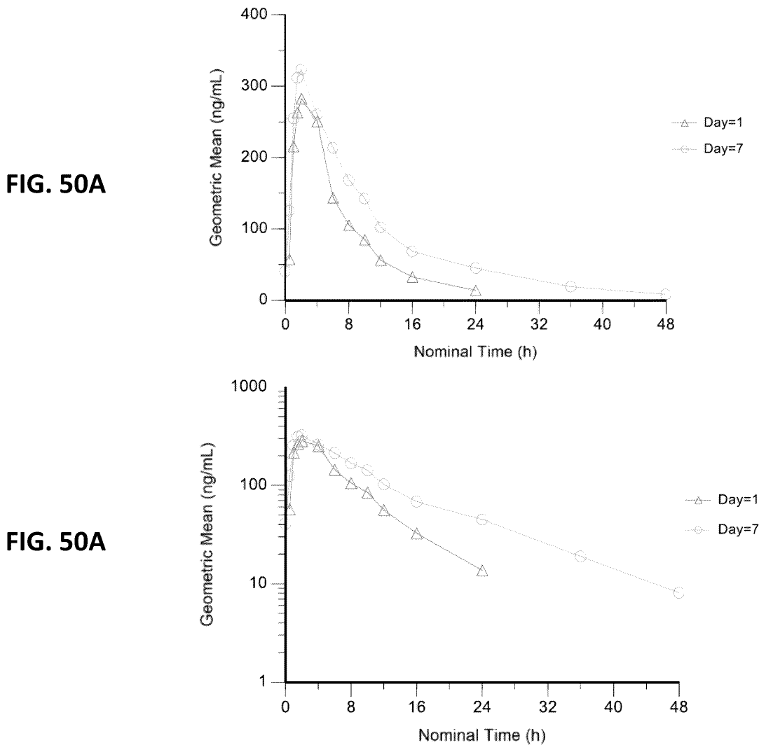

Maximum plasma TQS-168 concentrations on Day 1 occurred between 0.5 and 2.0 hours post-dose, with a median Tmax of 1.0 hours post-dose. Geometric mean (CV %) Cmax and AUC(0-tau) values 172 ng/mL (49.3%) and 438 ng*h/mL (55.1%), respectively. Maximum plasma TQS-621 concentrations on Day 1 occurred between 1.0 and 4.0 hours post-dose, with a median Tmax of 1.5 hours post-dose. Geometric mean (CV %) Cmax and AUC(0-tau) values 331 ng/mL (40.3%) and 2270 ng*h/mL (35.7%), respectively. See FIGS. 49-50.

Following multiple administrations, Day 7 plasma concentrations of TQS-168 and metabolite TQS-621 were quantifiable at pre-dose in all but one subject who became quantifiable at 0.5 hours post dose. The subjects all remained quantifiable up to between 16 and the final sampling time point of 48 hours post-dose.

Maximum plasma TQS-168 concentrations on Day 7 occurred between 0.5 and 1.5 hours post-dose, with a median Tmax of 0.75 hours post-dose. Concentrations then declined yielding a mean elimination half-life of 7.7 hours. Geometric mean (CV %) Cmax and AUC(0-tau) values were 273 ng/mL (105.8%) and 692 ng*h/mL (89.8%), respectively. Geometric mean (CV %) accumulation ratios were 1.59 (88.1%) and 1.58 (51.6%), based on Cmax and AUC(0-tau), respectively. See FIGS. 49-50.

Maximum plasma TQS-621 concentrations on Day 7 occurred between 1.0 and 4.0 hours post-dose, with a median Tmax of 1.75 hours post-dose. Concentrations then declined yielding a mean elimination half-life of 10.2 hours. Geometric mean (CV %) Cmax and AUC(0-tau) values were 340 ng/mL (39.5%) and 3320 ng*h/mL (42.1%), respectively. Geometric mean (CV %) accumulation ratios were 1.30 (38.7%) and 1.46 (30.8%), based on Cmax and AUC(0-tau), respectively. See FIGS. 49-50.

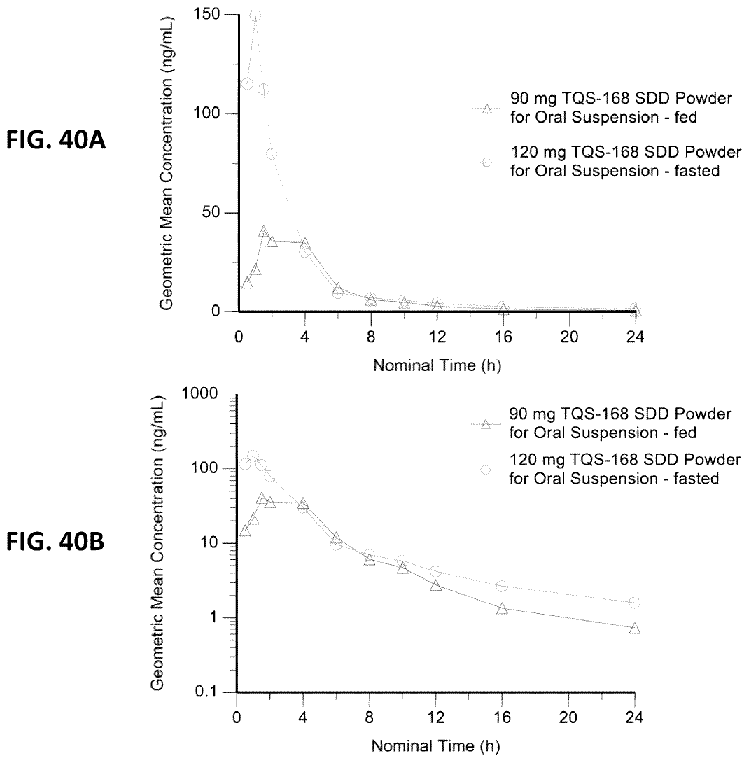

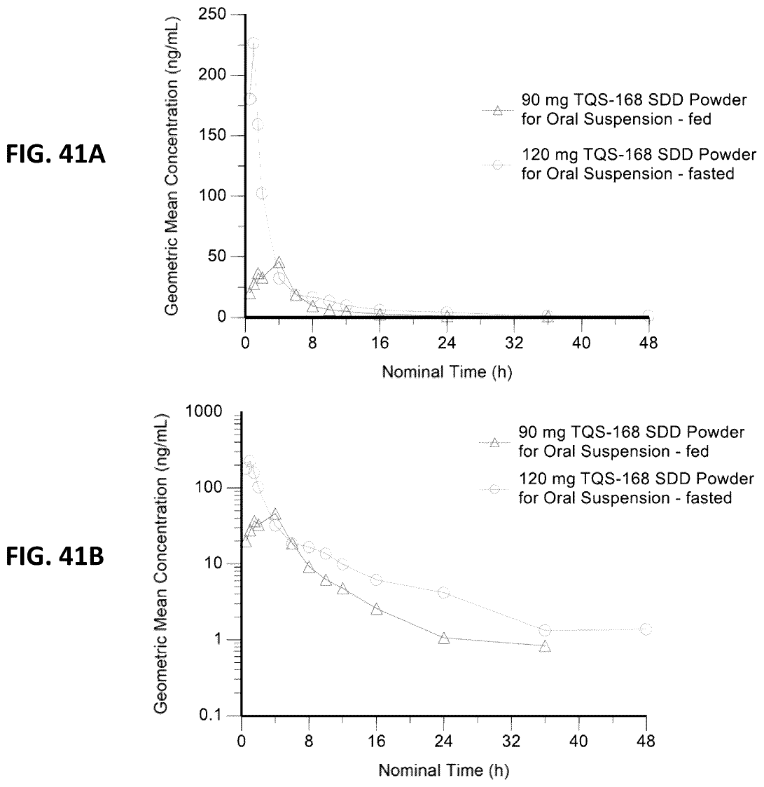

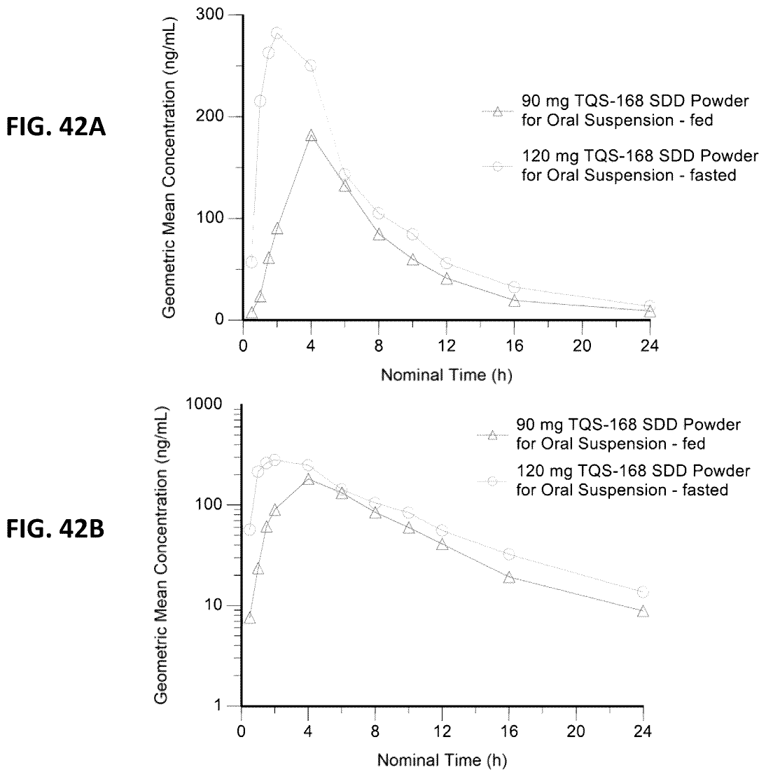

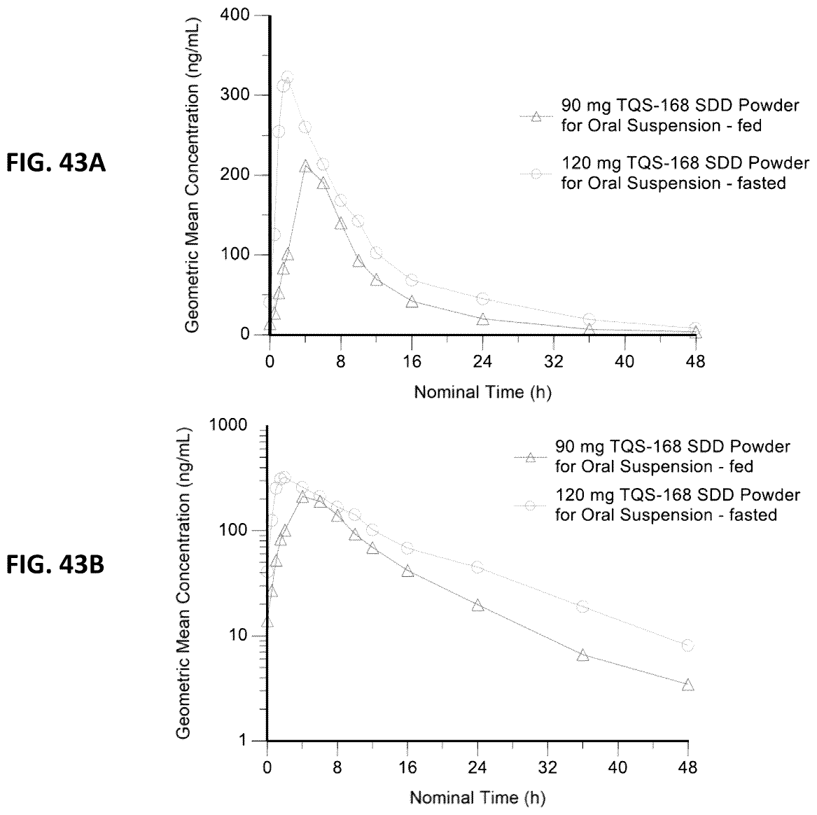

Regimen I PK summary, Subjects in Cohort 1 were administered Regimen I and received 120 mg TQS-168 SDD QD for seven consecutive days in the fasted state. Day 1 provided a TQS-168 Cmax of 172 ng/mL (0.69 μM). Day 7 of consecutive dosing provided a much greater TQS-168 Cmax of 273 ng/mL (1.09 μM). Results are plotted in FIGS. 40-41. Similar increases were observed in AUC measured exposure (Day 1 AUC(0-tau)=438 hr*ng/mL, Day 7 AUC(0-tau)=692 hr*ng/mL). Metabolite TQS-621 PK profile: Cohort 1 displayed a Day 1 metabolite TQS-621 Cmax of 331 ng/mL (1.24 μM) and a Day 7 Cmax of 340 ng/mL (1.27 μM). Results are plotted in FIGS. 42-43. A metabolite exposure of AUC(0-tau)=2270 hr*ng/mL was recorded for Day 1. Day 7 provided a metabolite exposure of AUC(0-tau)=3220 hr*ng/m. See FIGS. 49-50.

TQS-168 Regimen J PK profile: Subjects in Cohort 2 received a single 90 mg TQS-168 SDD p.o. QD in the fed state for seven consecutive days.

On Day 1, following a single administration of 90 mg TQS-168 Spray Dried Dispersion (SDD) Powder for Oral Suspension, plasma concentrations of TQS-168 were quantifiable from 0.5 hours post-dose for all subjects, and remained quantifiable up to between 16 and 24 hours post-dose. Concentrations of TQS-621 on Day 1 were also quantifiable from 0.5 h post-dose and remained quantifiable up to 24 hours post-dose in all subjects.

Maximum plasma TQS-168 concentrations on Day 1 occurred between 1.5 and 4.0 hours post-dose, with a median Tmax of 3.0 hours post-dose. Geometric mean (CV %) Cₘₐₓ and AUC(0-tau) values were 47.4 ng/mL (27.4%) and 223 ng·h/mL (30.9%), respectively. Comparison with Regimen I illustrated in FIG. 40A-B

Maximum plasma TQS-621 concentrations on Day 1 occurred between 4.0 and 6.0 hours post-dose, with a median Tmax of 4.0 hours post-dose. Geometric mean (CV %) Cₘₐₓ and AUC(0-tau) values were 189 ng/mL (40.1%) and 1400 ng*h/mL (42.6%), respectively. Comparison with Regimen I illustrated in FIG. 42A-B

On Day 1, no individual subject exceeded the maximum permitted Cₘₐₓ or the maximum permitted AUC(0-24) (based AUC(0-tau), where tau=24 h) values. The maximum individual TQS-168 Cmax on Day 1 was 65.8 ng/mL, which accounted for 21.6% of the Cmax exposure limit. The maximum individual TQS-168 AUC(0-tau) was 299 ng*h/mL, which accounted for 10.9% of the AUC(0-24) exposure limit.

Following multiple administrations of 90 mg TQS-168 Spray Dried Dispersion (SDD) Powder for Oral Suspension to healthy subjects in the fed state for 7 days, plasma concentrations of TQS-168 were quantifiable at pre-dose in all but three subjects who became quantifiable at 0.5 hours post-dose, and remained quantifiable up to between 16 and 36 hours post-dose. Concentrations of TQS-621 were quantifiable at the pre-dose time-point in all subjects, and remained quantifiable up to the final sampling time point of 48 hours post-dose.

Maximum plasma TQS-168 concentrations on Day 7 occurred between 1.0 and 4.0 hours post-dose, with a median Tmax of 3.0 hour post-dose. geometric mean elimination half-life of 4.46 h. Geometric mean (CV %) Cmax and AUC(0-tau) values were 48.8 ng/mL (48.3%) and 227 ng·h/mL (46.7%) respectively. Geometric mean (CV %) accumulation ratios were 1.03 (37.5%) and 1.24 (22.8%), based on Cmax and AUC(0-tau), respectively. Comparison with Regimen I illustrated in FIG. 41A-B

Maximum plasma TQS-621 concentrations on Day 7 occurred between 4.0 and 6.0 hours post-dose, with a median Tmax of 5.0 hours post-dose. Concentrations then declined in a generally biphasic manner giving rise to a geometric mean elimination half-life of 8.65 h. Geometric mean (CV %) Cmax and AUC(0-tau) values were 218 ng/mL (37.6%) and 2060 ng·h/mL (54.8%) respectively. Geometric mean (CV %) accumulation ratios were 1.15 (12.4%) and 1.47 (18.0%), based on Cmax and AUC(0-tau), respectively. Comparison with Regimen I illustrated in FIG. 43A-B

On Day 7, no individual subjects exceeded the maximum permitted Cmax value for TQS-168 or the maximum permitted AUC(0-24) (based AUC(0-tau), where tau=24 h values. The maximum individual TQS-168 Cmax on Day 7 was 94.1 ng/mL, which accounted for 30.9% of the Cmax exposure limit. The maximum individual TQS-168 AUC(0-tau) was 535 ng·h/mL, which accounted for 19.5% of the AUC(0-24) exposure limit.

Regimen J PK summary: subjects in Cohort 2 were administered Regimen J and received a once a day dose of 90 mg TQS-168 SDD in the fed state and provided a Day 1 TQS-168 Cmax of 47.4 ng/mL (0.19 μM). On Day 7 of consecutive dosing with 90 mg TQS-168 SDD in the fed state, a TQS-168 Cmax of 48.8 ng/mL (0.19 μM) was observed. Results are plotted in FIGS. 40-41. Similar figures were observed in comparison of the exposure profile (Day 1 AUC(0-tau)=223 hr*ng/mL, Day 7 AUC(0-tau)=227 hr*ng/mL. Regards TQS-621 PK profile, Cohorts displayed a Day 1 metabolite TQS-621 Cmax of 189 ng/mL (0.71 μM) and a Day 7 Cₘₐₓ of 218 ng/mL (0.82 μM). Results are plotted in FIGS. 42-43. A metabolite exposure of AUC(0-tau)=1400 hr*ng/mL was recorded for Day 1. Day 7 provided a metabolite exposure of AUC(0-tau)=2060 hr*ng/m.

TQS-168 Regimen K PK profile: Subjects in Cohort 3 received a single 300 mg TQS-168 SDD QD in the fed state for seven consecutive days.

On Day 1, following a single administration of 300 mg TQS-168 Spray Dried Dispersion (SDD) Powder for Oral Suspension, plasma concentrations of TQS-168 were quantifiable from 0.5 hours post-dose for all subjects, and remained quantifiable up to the final sampling point of 24 h post dose in all subjects.

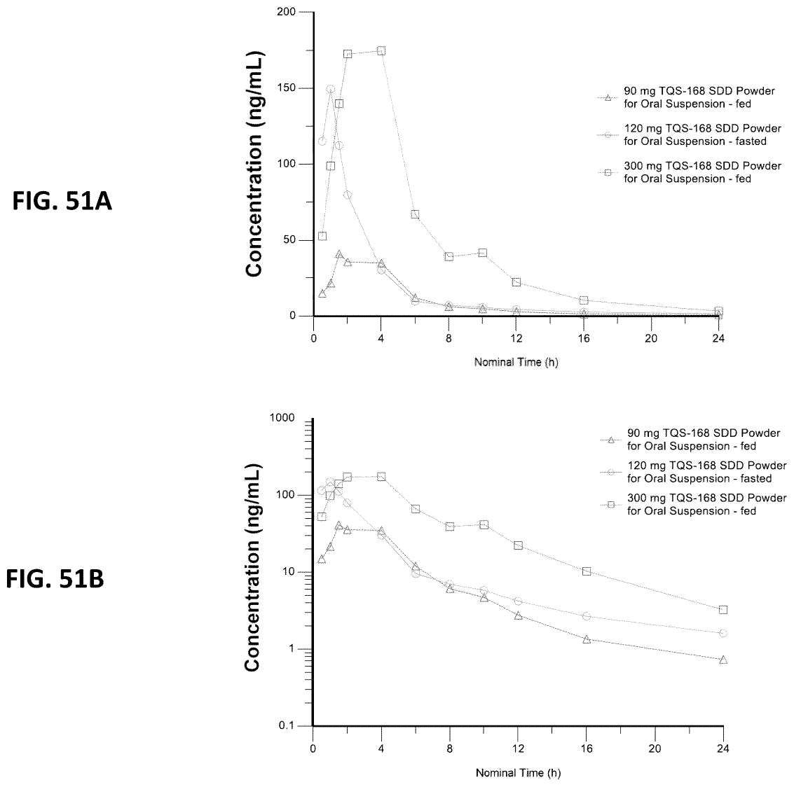

Maximum plasma TQS-168 concentrations on Day 1 occurred between 1.5 and 4.0 hours post-dose with a median Tmax of 2.0 hours post-dose. Geometric mean (CV %) Cₘₐₓ and AUC(0-tau) values were 229 ng/mL (38.3%) and 1210 ng*h/mL (55.1), respectively. See FIGS. 51A-B for illustrated comparison with Regimen I and J.

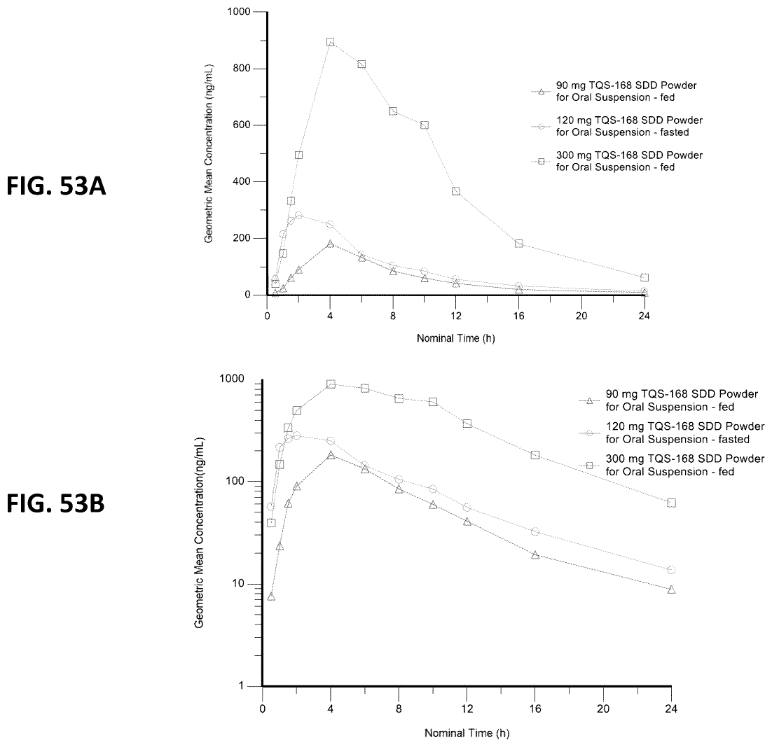

Maximum plasma TQS-621 concentrations on Day 1 occurred between 4.0 and 10.0 hours post-dose, with a median Tmax of 4.0 hours post-dose. Geometric mean (CV %) Cmax and AUC(0-tau) values 1000 ng/mL (24.6%) and 9730 ng*h/mL (39.9%), respectively. See FIGS. 53A-B for illustrated comparison with Regimen I and J.

Following multiple administrations Day 7 plasma concentrations of TQS-168 and metabolite TQS-621 were quantifiable at pre-dose in all subjects and remained quantifiable up to between 24 and 48 hours post-dose. Concentrations of TQS were also quantifiable at the pre-dose time-point in all subjects and remained quantifiable up the final sampling time point of 48 hours post-dose.

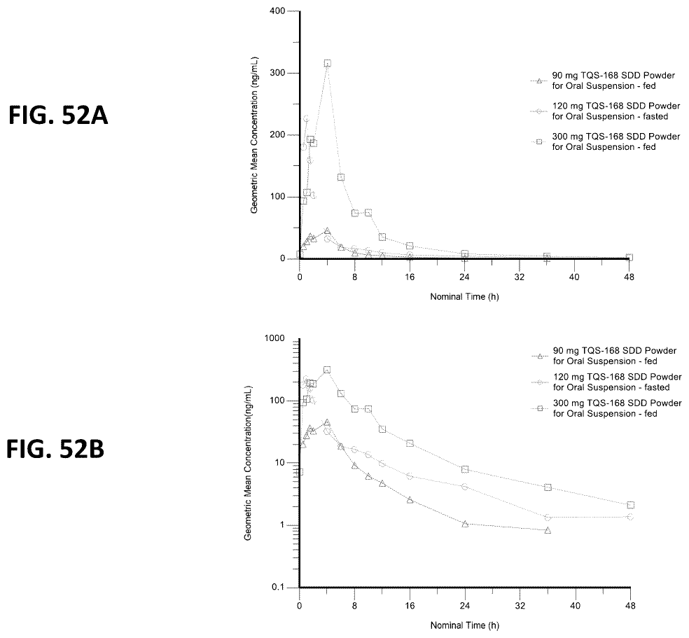

Maximum plasma TQS-168 concentrations on Day 7 occurred between 0.50 and 4.0 hours post-dose, with a median Tmax of 4.0 hours post-dose. Concentrations then declined yielding a mean elimination half-life of 5.67 hours. Geometric mean (CV %) Cmax and AUC(0-tau) values were 400 ng/mL (79.3%) and 2010 ng*h/mL (88.3%), respectively. Geometric mean (CV %) accumulation ratios were 1.74 (54.6%) and 1.66 (29.5%), based on Cmax and AUC(0-tau), respectively. See FIGS. 52A-B for illustrated comparison with Regimen I and J.

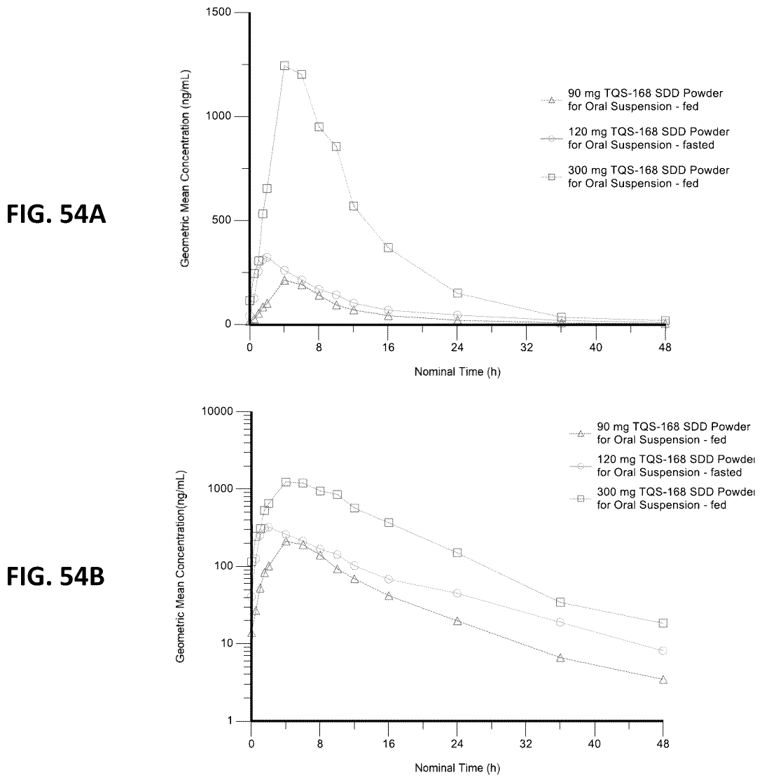

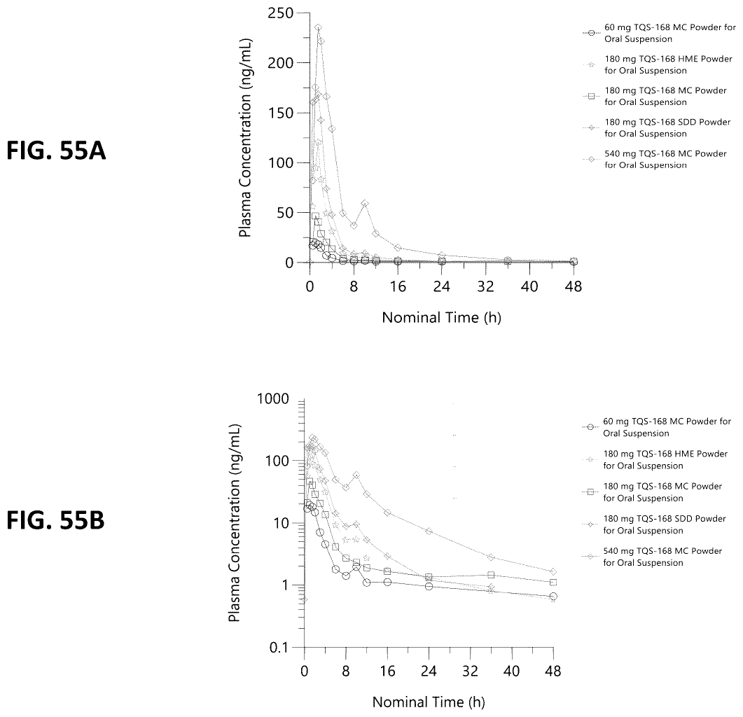

Maximum plasma TQS-621 concentrations on Day 7 occurred between 4.0 and 6.0 hours post-dose, with a median Tmax of 5.0 hours post-dose. Concentrations then declined yielding a mean elimination half-life of 7.29 hours. Geometric mean (CV %) Cmax and AUC(0-tau) values were 1300 ng/mL (36.7%) and 14900 ng*h/mL (61.2%), respectively. Geometric mean (CV %) accumulation ratios were 1.30 (32.9%) and 1.53 (26.1%), based on Cmax and AUC(0-tau), respectively. See FIGS. 54A-B for illustrated comparison with Regimen I and J.

Regimen K PK summary, subjects in Cohort 1 were administered Regimen K and received 300 mg TQS-168 SDD QD for seven consecutive days in the fed state. Day 1 provided a TQS-168 Cmax of 229 ng/mL (0.91 μM). Day 7 of consecutive dosing provided a much greater TQS-168 Cmax of 400 ng/mL (1.60 μM). Results are plotted in FIGS. 40-41. Similar increases where observed in AUC measured exposure (Day 1 AUC(0-tau)=1210 hr*ng/mL, Day 7 AUC(0-tau)=2010 hr*ng/mL). Cohort 1 displayed a Day 1 metabolite TQS-621 Cₘₐₓ of 1000 ng/mL (3.76 μM) and a Day 7 Cₘₐₓ of 1300 ng/mL (4.88 μM). Results are plotted in FIGS. 42-43. A metabolite exposure of AUC(0-tau)=9730 hr*ng/mL was recorded for Day 1. Day 7 provided a metabolite exposure of AUC(0-tau)=14900 hr*ng/m.

In summary, Part 2 of this trial provided healthy male subjects with consecutive seven QD doses of TQS-168 in the fed or fasted state at different dosages of TQS-168 SDD powder for oral suspension. The data reveals that increase in dosage corresponds to an increase in plasma concentration of TQS-168 and TQS-168 metabolite TQS-621.

### 6. EQUIVALENTS AND INCORPORATION BY REFERENCE

While the invention has been particularly shown and described with reference to a preferred embodiment and various alternate embodiments, it will be understood by persons skilled in the relevant art that various changes in form and details can be made therein without departing from the spirit and scope of the invention.

All references, issued patents and patent applications cited within the body of the instant specification are hereby incorporated by reference in their entirety, for all purposes.

1. A method of reducing neuroinflammation and/or treating a neurodegenerative disease in a human subject, the method comprising:
orally administering to a subject with neuroinflammation and/or a neurogenerative disease at least one dose of a pharmaceutical composition comprising the compound of formula (I) 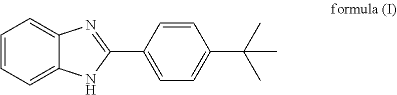 [SMILES: CC(C)(C)c1ccc(-c2nc3ccccc3[nH]2)cc1]
(TQS-168), or a pharmaceutically acceptable salt thereof, in an amount that provides, after administration,
a mean peak concentration (Cₘₐₓ) of TQS-168 in plasma of at least 750 ng/mL.

2. The method of claim 1, wherein TQS-168 or salt thereof is administered in an amount that provides, following administration, a mean plasma Cmax of TQS-168 of at least 1000 ng/mL.

3. The method of claim 2, wherein TQS-168 or salt thereof is administered in an amount that provides, following administration, a mean plasma Cmax of TQS-168 of at least 1250 ng/mL.

4. The method of claim 3, wherein TQS-168 or salt thereof is administered in an amount that provides, following administration, a mean plasma Cmax of TQS-168 of at least 1500 ng/mL.

5. The method of claim 4, wherein TQS-168 or salt thereof is administered in an amount that provides, following administration, a mean plasma Cₘₐₓ of TQS-168 of at least 1750 ng/mL.

6. The method of claim 1, wherein TQS-168 or salt thereof is administered in an amount that provides, following administration, a TQS-168 AUC₀₋ₜ of at least 3000 ng·hr/ml.

7. The method of claim 6, wherein TQS-168 or salt thereof is administered in an amount that provides, following administration, a TQS-168 AUC₀₋ₜ of at least 4000 ng·hr/ml.

8. The method of claim 7, wherein TQS-168 or salt thereof is administered in an amount that provides, following administration, a TQS-168 AUC₀₋ₜ of at least 5000 ng·hr/ml.

9. The method of claim 8, wherein TQS-168 or salt thereof is administered in an amount that provides, following administration, a TQS-168 AUC₀₋ₜ of at least 5500 ng·hr/ml.

10. The method of claim 9, wherein TQS-168 or salt thereof is administered in an amount that provides, following administration, a TQS-168 AUC₀₋ₜ of at least 6000 ng·hr/ml.

11. The method of claim 10, wherein TQS-168 or salt thereof is administered in an amount that provides, following administration, a TQS-168 AUC₀₋ₜ of about 7000 ng·hr/ml.

12. The method of claim 1, wherein the time to plasma Cmax (Tmax) of TQS-168 is no more than 2 hours.

13. The method of claim 12, wherein the Tmax is no more than 90 minutes.

14. The method of claim 13, wherein the Tmax is no more than 75 minutes.

15. The method of claim 14, wherein the Tmax is about 60 minutes.

16. A method of reducing neuroinflammation and/or treating a neurodegenerative disease in a human subject, the method comprising:
orally administering to a subject with neuroinflammation and/or a neurogenerative disease a pharmaceutical composition comprising the compound of formula (I) 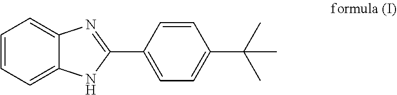 [SMILES: CC(C)(C)c1ccc(-c2nc3ccccc3[nH]2)cc1]
(TQS-168), or a pharmaceutically acceptable salt thereof, in an amount that provides following administration,
a mean peak plasma concentration (Cₘₐₓ) of the compound of Formula (II) 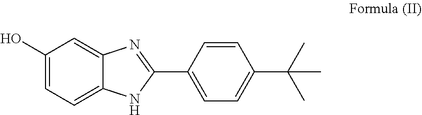 [SMILES: CC(C)(C)c1ccc(-c2nc3cc(O)ccc3[nH]2)cc1]
(TQS-621) of at least 1000 ng/mL.

17-19. (canceled)

20. A method of reducing neuroinflammation and/or treating a neurodegenerative disease in a human subject, the method comprising:
orally administering to a subject with neuroinflammation and/or a neurogenerative disease a pharmaceutical composition comprising the compound of formula (I) 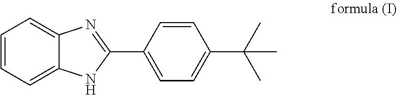 [SMILES: CC(C)(C)c1ccc(-c2nc3ccccc3[nH]2)cc1]
(TQS-168), or a pharmaceutically acceptable salt thereof, in an amount that provides following administration,
(a) a mean peak concentration (Cₘₐₓ) of TQS-168 in plasma of at least 750 ng/mL, with
(b) a mean time to Cₘₐₓ (Tₘₐₓ) of TQS-168 in plasma of no more than 75 minutes; and
(c) a mean peak concentration (Cₘₐₓ) of the compound of Formula (II) 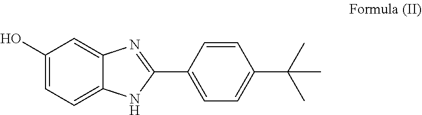 [SMILES: CC(C)(C)c1ccc(-c2nc3cc(O)ccc3[nH]2)cc1]
(TQS-621) in plasma of at least 1000 ng/mL, with
(d) a mean time to Cₘₐₓ (Tₘₐₓ) of TQS-621 in plasma of no more than 4 hours.

21. The method of claim 1, wherein TQS-168, or salt thereof, is administered in a daily oral dose of 200-800 mg.

22. The method of claim 21, wherein TQS-168, or salt thereof, is administered in a daily oral dose of 300-700 mg.

23. The method of claim 22, wherein TQS-168, or salt thereof, is administered in a daily oral dose of 400-600 mg.

24. The method of claim 23, wherein TQS-168, or salt thereof, is administered in a daily oral dose of 400-500 mg.

25. The method of claim 24, wherein TQS-168, or salt thereof, is administered in a daily oral dose of 400 mg or 450 mg.

26. The method of claim 1, wherein TQS-168 or salt thereof is administered in a liquid suspension

27. The method of claim 1, wherein TQS-168 or salt thereof is administered in a liquid solution.

28. The method of claim 1, wherein TQS-168 or salt thereof is administered in a solid dosage form.

29. The method of claim 28, wherein TQS-168 or salt thereof is crystalline.

30. The method of claim 28, wherein TQS-168 or salt thereof is amorphous.

31. The method of claim 30, wherein TQS-168 or salt thereof is in the form of a spray-dried dispersion.

32. The method of claim 30, wherein TQS-168 of salt thereof is in the form of a hot melt extrusion.

33. The method of claim 28, wherein the solid dosage form is a sachet.

34. The method of claim 28, wherein the solid dosage form is a capsule.

35. The method of claim 28, wherein the solid dosage form is a tablet.

36. The method of claim 1, wherein the subject has a neurodegenerative disease selected from a motor neuron disease, amyotrophic lateral sclerosis (ALS), Alzheimer's disease, vascular dementia, frontotemporal degeneration (frontotemporal dementia), dementia with Lewy bodies, Parkinson's disease, Huntington's disease, demyelinating disease, and multiple sclerosis (MS).

37-42. (canceled)
